SEQUENCE LISTING (1 sequence)

SEQ ID NO: 1 - protein, 5 aa, unidentified
LPXTG

(19) United States
(12) Patent Application Publication
(10) Pub. No.: US 2025/0235404 A1
(43) Pub. Date: Jul. 24, 2025

(54) PEG-LIPIDS AND LIPID NANOPARTICLES

(71) Applicant: CAPSTAN THERAPEUTICS, INC., San Diego, CA (US)

(72) Inventors: Priya Prakash Karmali, San Diego, CA (US); Steven Tanis, Carlsbad, CA (US)

(21) Appl. No.: 18/853,665

(22) PCT Filed: Apr. 5, 2023
(86) PCT No.: PCT/US2023/017648

§ 371 Date: Oct. 2, 2024

Related U.S. Application Data
(60) Provisional application No. 63/362,502, filed on Apr. 5, 2022.

(51) Int. Cl. A61K 9/1271; A61K 9/51; C07C 233/18; C07D 207/24; C12N 15/88

(52) U.S. Cl. CPC A61K 9/1271; A61K 9/5123; C07C 233/18; C07D 207/24; C12N 15/88

(57) ABSTRACT

Disclosed herein are polyethylene glycol (PEG)-lipids, functionalized PEG-lipids, and functionalized PEG-lipids that are conjugated to a binding moiety which can comprise an antibody antigen binding domain. Also disclosed are methods for synthesizing and functionalizing the PEG-lipids. The PEG-lipids are useful components lipid nanoparticles (LNP) used for the delivery of nucleic acids into living cells, in vivo or ex vivo. LNP comprising functionalized PEG-lipids that are conjugated to a binding moiety are useful as targeted LNP for delivering nucleic acids into cells or tissues expressing the ligand of the binding moiety.

### RELATED APPLICATIONS

This application claims priority to U.S. Provisional Patent Application No. 63/362,502, filed on Apr. 5, 2022, the entire contents of which is herein incorporated by reference.

### BACKGROUND

Lipid formulations have been used in the laboratory for the delivery of nucleic acids into cells. Early formulations based on the cationic lipid 1,2-dioleoyl-3-trimethylammonium propane (DOTAP) and the ionizable, fusogenic lipid dioleoylphosphatidyl ethanolamine (DOPE) had a large particle size and were problematic when used in vivo, exhibiting too rapid clearance, tropism for the lung, and toxicity. Lipid nanoparticles (LNPs) comprising ionizable cationic lipids have been developed to address these issues to the extent that RNA based products, such as the siRNA ONPATTRO® and two mRNA-based SARS-CoV-2 vaccines have received regulatory approval and entered the market. There is limited ability to control which tissues or cells take up the LNP once administered. LNP administered intravenously are taken up primarily in the liver, lung, or spleen depending to a significant degree on net charge and particle size. It is possible to direct >90% of LNP to the liver by a combination of formulation and intravenous administration. Intramuscular administration can provide a clinically useful level of local delivery and expression. LNP can be redirected to other tissues or cell types by conjugating a binding moiety with specificity for the target tissue or cell type, for example, conjugating an antigen binding domain from an antibody, to the LNP. Nonetheless, avoiding uptake by the liver remains a challenge. Moreover, with current systems only a minor portion of the encapsulated nucleic acid is successfully delivered to the cells of interest and into the cytoplasm. Current formulations may release only 2-5% of the administered RNA into the cytoplasm (see for example Gilleron et al., Nat. Biotechnol. 31:638-646, 2013, and Munson et al., Commun. Biol. 4:211-224, 2021). Thus, there are remaining issues of off-target delivery, poor efficiency of release of nucleic acid into the cytoplasm, and toxicity associated with accumulation of the component lipids.

Therefore, this disclosure provides Polyethylene glycol-lipids (PEG-lipids), improved conjugations chemistries and targeted lipid nanoparticles to satisfy an urgent need in the field.

### SUMMARY

Disclosed herein are certain PEG-lipids, functionalized PEG-lipids, lipid nanoparticles (LNP) comprising the PEG-lipids and/or functionalized PEG-lipids, and targeted LNP (tLNP) comprising functionalized PEG-lipid that has been conjugated to a binding moiety. Also disclosed are methods for synthesizing, functionalizing, and conjugating the PEG-lipids, as well as intermediates useful in synthesis of these lipids and methods of synthesizing the intermediates. The PEG-lipids and functionalized PEG-lipids are useful components of lipid nanoparticles (LNP) used for the delivery of nucleic acids into living cells, in vivo or ex vivo. LNP compositions comprising the functionalized PEG-lipid enable conjugation of a binding moiety, so as to become tLNP, that is, LNP in which a binding moiety has been conjugated to the functionalized lipid to serve as a targeting moiety to direct the tLNP to a desired tissue or cell type.

One aspect is a symmetrical tri-ester PEG-lipid, and functionalized PEG-lipid thereof, in which an esterified PEG moiety is attached to a central position on a scaffold and two identical fatty acids are esterified to two end positions on the scaffold. In some embodiments, the scaffold has the structure of Formula S1

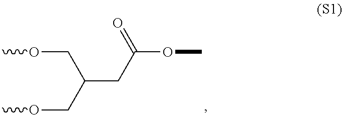

where [symbol] represents the points of ester connection with a fatty acid and [symbol] represents the point of ester formation with the PEG moiety. In some embodiments, the fatty acid esters are C₁₄-C₂₀ straight-chain alkyl fatty acids. In some embodiments, the PEG moiety is functionalized and the fatty acid esters are C₁₆-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. By symmetric it is meant that each alkyl branch has the same number of carbons. In some embodiments, the branch is at the 3, 4, 5, 6, or 7 position of the fatty acid ester.

One aspect is a symmetrical di-ester PEG-lipid, and functionalized PEG-lipid thereof, in which a PEG-moiety is attached to a central position on a scaffold by an ether linkage and two identical fatty acids are esterified to two end positions on the scaffold. In some embodiments, the scaffold has the structure of Formula S2

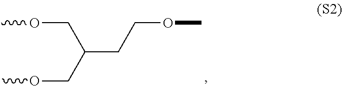

where [symbol] represents the points of esterification with a fatty acid, and [symbol] represents the point of ether formation with the PEG moiety. In some embodiments, the fatty acid esters are C₁₄-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the PEG moiety is functionalized and the fatty acid esters are C₁₆-C₂₀ straight-chain alkyl fatty acids, e.g., C₁₆, C₁₇, C₁₈, C₁₉, or C₂₀ straight-chain alkyl fatty acids. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. In some embodiments, the branch is at the 3, 4, 5, 6, or 7 position of the fatty acid ester.

In other embodiments of the symmetrical di-ester PEG-lipid, and functionalized PEG-lipid thereof, in which a PEG-moiety is attached to a central position on a glycerol scaffold by an ether linkage and two identical fatty acids are esterified to two end positions on the glycerol scaffold, having the structure of Formula S3

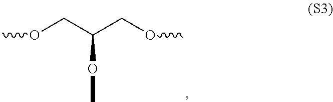

where [symbol] represents the points of esterification with a fatty acid, and [symbol] represents the point of ether formation with the PEG moiety. In some embodiments, the fatty acid esters are C₁₄-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the PEG moiety is functionalized and the fatty acid esters are C₁₆-C₂₀ straight-chain alkyl fatty acids, e.g., C₁₆, C₁₇, C₁₈, C₁₉, or C₂₀ straight-chain alkyl fatty acids. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. In some embodiments, the branch is at the 3, 4, 5, 6, or 7 position of the fatty acid ester. In some embodiments, the symmetrical di-ester PEG-lipid with a glycerol scaffold is Compound B-21, VI-7, or Compound B-23. In some embodiments, the symmetrical di-ester PEG-lipid with a glycerol scaffold is Compound B-22, VI-8 or Compound B-24.

One aspect is an asymmetric glycerol-based PEG-lipid, and functionalized PEG-lipid thereof, in which the glycerol scaffold has the structure of Formula S4

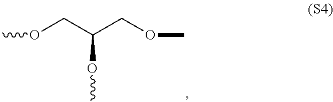

or the enantiomer or racemic mixture thereof, where [symbol] represents the points of esterification with a fatty acid, and [symbol] represents the point of ether formation with the PEG moiety, comprising two identical symmetrically branched fatty acids that each have a total carbon count of C₁₄-C₂₀. For example, the branched fatty acid is C₁₄, C₁₅, C₁₆, C₁₇, C₁₈, C₁₉, or C₂₀. In this aspect, the PEG moiety is attached at the position of one of glycerol's primary hydroxyls groups by an ether linkage. Embodiments of this aspect include Compounds B-31, B-33, and B-35.

One aspect is a method of synthesizing a symmetrical tri-ester PEG-lipid on a scaffold of formula S1, and functionalized PEG-lipid thereof, in which an esterified PEG moiety is attached to a central position and two identical fatty acids are esterified to two end positions on the scaffold, e.g., according to the synthetic scheme of FIGS. 1A-1B, 2A-2B, 3A-3C, 4A-4C, 11A-11B, and 12.

One aspect is a method of synthesizing a symmetrical di-ester PEG-lipid, and functionalized PEG-lipid thereof, having a scaffold of formula S2, e.g., according to the synthetic scheme of FIGS. 5A-5D, 6A-6C, and 12.

One aspect is a method of synthesizing a symmetrical di-ester PEG-lipid, and functionalized PEG-lipid thereof, comprising a symmetric glycerol-derived scaffold of formula S3 in which an esterified PEG moiety is attached to a central position and two identical fatty acids are esterified to two end positions of the scaffold, e.g., according to the synthetic scheme of FIGS. 7A-7B, 8A-8C, and 12.

One aspect is a method of synthesizing an asymmetric glycerol-based PEG-lipid, and functionalized PEG-lipid thereof, comprising an asymmetric glycerol-derived scaffold of formula S4, or the enantiomer or racemic mixture thereof, with 2 identical fatty acids that are C₁₄-C₂₀ symmetric branched-chain alkyl fatty acids, e.g., according to the synthetic scheme of FIGS. 9A-9F, 10A-10C, and 12.

Examples of is a functionalized PEG-lipid comprise, without limitation, a bromomaleimide or bromomaleimide amide moiety, an alkynylamide moiety, or an alkynylimide moiety at the terminal hydroxyl end of the PEG moiety.

One aspect is a LNP comprising one or more PEG-lipids selected from the group consisting of symmetrical PEG-lipids in which a PEG-moiety is attached to a central position on a scaffold and two identical fatty acids are esterified to two end positions on the scaffold and asymmetric glycerol-based PEG-lipids. In some embodiments, the one or more PEG-lipids can be the same or different. In some embodiments, the symmetrical PEG-lipids respectively and independently may comprise a scaffold selected from the group consisting of Formulas S1, S2, and S3. In some embodiments, the asymmetrical PEG-lipids respectively and independently may comprise a scaffold of S4. In some embodiments, the one or more PEG-lipids may respectively and independently have a structure selected form the group consisting of Formulas PL-1, PL-2, PL-3, and PL-4. In certain embodiments, the LNP further comprises one or more ionizable cationic lipids respectively and independently having a structure selected from the group consisting of Formulas 1, 2, and 3. In certain embodiments, the LNP is a tLNP comprising one or more functionalized PEG-lipids that has been conjugated to a binding moiety. In certain embodiments, one or more of the one or more functionalized PEG-lipids are respectively and independently selected from the group consisting of symmetrical PEG-lipids and asymmetric glycerol-based PEG-lipids. In certain embodiments, one or more of the one or more functionalized PEG-lipids respectively and independently have a structure selected from the group consisting of Formulas PL-1, PL-2, PL-3, and PL-4. In some embodiments the functionalization is a bromomaleimide or bromomaleimide amide, alkynylamide, or alkynylimide. In some embodiments, the binding moiety comprises an antibody or antigen binding portion thereof. In some embodiments, the binding moiety is a polypeptide comprising a binding domain and an N- or C-terminal extension comprising an accessible thiol group.

One aspect is a tLNP comprising a disclosed herein PEG-lipid conjugated to a binding moiety. In some embodiments, the conjugation linkage comprises a reaction product of a thiol in the binding moiety with a functionalized PEG-lipid. In some embodiments the functionalization is a maleimide, azide, alkyne, dibenzocyclooctyne (DBCO), bromomaleimide or bromomaleimide amide, alkynylamide, or alkynylimide. In some embodiments, the binding moiety comprises an antibody or antigen binding portion thereof. In some embodiments, the binding moiety is a polypeptide comprising a binding domain and an N- or C-terminal extension comprising an accessible thiol group.

With respect to the above aspects, in some embodiments, the PEG moiety is PEG-500 to PEG-5000 such as PEG-500, PEG-1000, PEG-1500, PEG-2000, PEG-2500, PEG-3000, PEG-3500, PEG-4000, PEG-4500, and PEG-5000. In some instances, the PEG moiety is PEG-2000.

With respect to any of the above aspects in which the PEG-lipids have not been functionalized, in some embodiments the distal end of the PEG moiety can terminate with a hydroxyl, a methoxyl, a benzyloxyl or a 4-methoxybenzyloxyl group.

With respect to any of the above aspects in which the PEG-lipids have been functionalized (but not conjugated), in some embodiments the distal end of the PEG moiety can terminate with a maleimide, azide, alkyne, DBCO, bromomaleimide or bromomaleimide amide, alkynylamide, or alkynylimide.

With respect to any of the LNP aspects, the LNP, including the tLNP, may further comprise one or more of an ionizable cationic lipid, a phospholipid, a sterol, a co-lipid, and a further PEG-lipid, or combinations thereof. In some embodiments, the further PEG-lipid is not functionalized or conjugated. In some embodiments, the herein disclosed PEG-lipids serve as the non-functionalized PEG-lipid, the functionalized or conjugated PEG-lipid, or both. As used herein, functionalized PEG-lipid refers to a PEG-lipid in which the PEG moiety has been derivatized with a chemically reactive group that can be used for conjugating a targeting moiety to the PEG-lipid. The functionalized PEG-lipid can be reacted with a binding moiety after the LNP is formed, so that the binding moiety is conjugated to the PEG portion of the lipid. The conjugated binding moiety can thus serve as a targeting moiety for the LNP to form a tLNP.

With respect to the LNP or the tLNP, in various embodiments, the phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof.

With respect to the LNP or the tLNP, in various embodiments, the sterol is cholesterol or a phytosterol, or a combination thereof. In further embodiments the phytosterol comprises campesterol, sitosterol, or stigmasterol, or combinations thereof.

With respect to the LNP or the tLNP, in various embodiments, the ionizable cationic lipid comprises a lipid with a measured pKa in the LNP of 6 to 7, facilitating ionization in the endosome. In some embodiments the ionizable cationic lipid has a c-pKa from 8 to 11 and cLogD from 9 to 18 or 11-14. In some embodiments, the ionizable cationic lipids have branched structure to give the lipid a conical rather than cylindrical shape. Suitable ionizable cationic lipids are known to those of skill in the art. In some preferred embodiments, the ionizable cationic lipid has a structure of Formula 1, Formula 2, or Formula 3, including species or subgenera thereof, as disclosed in U.S. Provisional Application Nos. 63/489,381 filed on Mar. 9, 2023, 63/366,462 filed Jun. 15, 2022, and 63/362,501 filed on Apr. 5, 2022, all entitled Ionizable Cationic Lipids and Lipid Nanoparticles (Atty. Docket No. 146758-8001.US00-02), and PCT application entitled Ionizable Cationic Lipids and Lipid Nanoparticles (Atty. Docket No. 146758-8001.WO00), filed on date even of this application, which are incorporated by reference in their entirety.

With respect to the LNP or the tLNP, in some embodiments, the co-lipid is absent or comprises an ionizable lipid. In some embodiments the ionizable lipid is cholesterol hemisuccinate (CHEMS). In some embodiments, the co-lipid is a charged lipid, such as a quaternary ammonium headgroup containing lipid. In some instances, the quaternary ammonium headgroup containing lipid comprises 1,2-dioleoyl-3-trimethylammonium propane (DOTAP), N-(1-(2,3-dioleyloxy)propyl)-N,N,N-trimethylammonium chloride (DOTMA), or 3p-(N-(N′,N′-Dimethylaminoethane)carbamoyl)cholesterol (DC-Chol), or combinations thereof. In addition to the chloride salts of the quaternary ammonium headgroup containing lipids, further instances include bromide, mesylate, and tosylate salts.

With respect to the LNP or the tLNP, in some embodiments, the further PEG-lipid (that is, a lipid conjugated to a polyethylene glycol (PEG)) is a C₁₄-C₂₀ lipid conjugated with a PEG. In some embodiments, the PEG is of 500-5000 Da molecular weight (MW) such as PEG-500, PEG-1000, PEG-1500, PEG-2000, PEG-2500, PEG-3000, PEG-3500, PEG-4000, PEG-4500, and PEG-5000. In some embodiments, the PEG unit has a MW of 2000 Da. In some instances, the MW2000 PEG-lipid comprises DMG-PEG2000 (1,2-dimyristoyl-glycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof. In some embodiments, the glycerol moiety is racemic. Alternatively, optically pure antipodes of the glycerol portion can be employed, that is, the glycerol portion is homochiral.

With respect to the LNP or the tLNP, in some embodiments the molar ratio of the lipids is 0.1 to 5 mol % functionalized PEG-lipid: 40 to 60 mol % ionizable cationic lipid: 7 to 30 mol % phospholipid: 20 to 45 mol % sterol: 1 to 30 mol % co-lipid, if present: 0 to 5 mol % further PEG-lipid, if present. In some embodiments, the functionalized PEG-lipid is conjugated to a binding moiety.

The LNP or the tLNP further comprises a nucleic acid. In various embodiments the nucleic acid is mRNA, self-replicating RNA, siRNA, miRNA, DNA, a gene editing component (for example, a guide RNA a tracr RNA, sgRNA, an mRNA encoding a gene or base editing protein, a zinc-finger nuclease, a Talen, a CRISPR nuclease, such as Cas9, a DNA molecule to be inserted or serve as a template for repair), and the like, or a combination thereof. In some embodiments the mRNA encodes a chimeric antigen receptor (CAR). In other embodiments the mRNA encodes a gene-editing or base-editing protein. In some embodiments, the nucleic acid is a guide RNA. In some embodiments, the LNP or tLNP comprises both a gene- or base-editing protein-encoding mRNA and one or more guide RNAs.

With respect to the LNP or the tLNP, in some embodiments the ratio of total lipid to nucleic acid is 10:1 to 50:1 on a weight basis. In some embodiments, that ratio of total lipid to nucleic acid is 10:1, 20:1, 30:1, or 40:1 to 50:1, or 10:1 to 20:1, 30:1, 40:1 or 50:1, or any range bound by a pair of these ratios.

One aspect is a method of making an LNP comprising rapid mixing of an aqueous solution of a nucleic acid and an alcoholic solution (for example, in ethanol) of the lipids.

One aspect is a method of making a tLNP comprising rapid mixing of an aqueous solution of a nucleic acid and an alcoholic solution of the lipids. In some embodiments, the lipid mixture includes functionalized PEG-lipid, for later conjugation to a targeting moiety. In other embodiments, the functionalized PEG-lipid is inserted into an LNP subsequent to formation of an initial LNP from other components. In either type of embodiment, the targeting moiety is conjugated to PEG-lipid after the PEG-lipid containing LNP is formed.

The aqueous solution used in the methods of making a LNP or tLNP can be buffered at a pH of ≤5. Once formed, the LNP can be stored, reconstituted, and/or administered in an aqueous solution buffered at a pH of 6 to 8.5 with Tris or HEPES and containing a salt, for example, NaCl. These aqueous solutions used during formation or for storage of the LNP or tLNP and may further comprise cryoprotectants such as glycerol and/or sugars (for example, sucrose, trehalose, or mannose) so that the LNP or tLNP may be stored frozen. Buffers can be exchanged through ultrafiltration or diafiltration.

In another aspect, newly disclosed herein is a method of delivering a nucleic acid into a cell comprising contacting the cell with LNP or tLNP of any of the forgoing aspects. In some embodiments the contacting takes place ex vivo. In some embodiments, the contacting takes place in vivo.

### BRIEF DESCRIPTION OF THE DRAWINGS

Unless specified otherwise, R¹ and n in the drawings are defined the same as in Formula PL-1.

FIGS. 1A-B depict a synthetic scheme for embodiments of symmetric tri-ester PEG-lipids PEG moiety built on a scaffold of formula S1. FIG. 1A depicts a synthetic scheme for symmetric tri-ester PEG-lipids of Formula B-1A. Specifically, FIG. 1B depicts synthesis of Compounds B-1 and B-2, which are embodiments of Formula B-1A, wherein the PEG moiety is PEG-2000, and R¹ is branched and straight-chain C₁₇ alkyl groups, respectively.

FIGS. 2A-B depict a synthetic scheme for bromomaleimide functionalization of the PEG moiety of the embodiments of the symmetric tri-ester PEG-lipids. FIG. 2A depicts a synthetic scheme for functionalization of the PEG moiety of PEG-lipids of Formula B-1A with bromomaleimide to provide bromomaleimide functionalized PEG-lipids of Formula B-3A. Specifically, FIG. 2B depicts conversion of Compounds B-1 and B-2, to the bromomaleimide functionalized PEG-lipids, Compounds B-3 and B-4, respectively.

FIGS. 3A-C depict a synthetic scheme for functionalization of the embodiments of symmetric tri-ester PEG-lipids' PEG moiety with a reactive alkynylimide. FIG. 3A shows the synthesis of intermediate IV-6. FIG. 3B shows the functionalization reaction using intermediate IV-6 to convert the PEG-lipids of Formula B-1A to functionalized PEG lipids of Formula B-5A. Specifically, FIG. 3C depicts conversion of Compounds B-1 and B-2, to the functionalized PEG-lipids, Compound B-5 and Compound B-6, respectively.

FIGS. 4A-C depict a synthetic scheme for functionalization of the embodiments of symmetric tri-ester PEG-lipids' PEG moiety with a reactive alkynylamide. FIG. 4A shows the synthesis of intermediate IV-8. FIG. 4B shows the functionalization reaction using intermediate IV-8 to convert the PEG-lipids of Formula B-1A to functionalized PEG lipids of Formula B-7A. Specifically, FIG. 4C depicts conversion of Compounds B-1 and B-2, to the functionalized PEG-lipids, Compound B-7 and Compound B-8, respectively.

FIGS. 5A-D depict a synthetic scheme for embodiments of symmetric di-ester PEG-lipids built on a scaffold of formula S2. FIG. 5A depicts a synthetic scheme for symmetric di-ester PEG-lipids of Formula B-9A, wherein the PEG moiety has a terminal methoxyl group. FIG. 5B depicts a synthesis scheme for symmetric di-ester PEG-lipids of Formula B-11 A, wherein the PEG moiety has a terminal 4-methoxybenzyloxyl group. Specifically, FIG. 5C depicts a synthesis scheme of Compounds B-9 and B-10, which are embodiments of Formula B-9A, wherein the PEG moiety is methoxy-PEG-2000, and R¹ is branched and straight-chain C₁₇ alkyl groups, respectively. FIG. 5D depicts a synthesis scheme of Compounds B-11 and B-12, which are embodiments of Formula B-11A, wherein the PEG moiety is 4-methoxylbenzyloxy-PEG-2000, and R¹ is branched and straight-chain C₁₇ alkyl groups, respectively.

FIGS. 6A-C depict the functionalization of the symmetric di-ester PEG-lipids of Formula B-13A. FIG. 6A shows the functionalization of PEG-lipids of Formula B-13A with bromomaleimde, IV-6, and IV-8 to produce functionalized PEG-lipids of Formulas B-15A, B-17A, and B-19A, respectively. Specifically, FIG. 6B shows the functionalization of Compound B-13, which is an embodiment of Formula B-13A, wherein the PEG moiety is PEG-2000, and R¹ is branched C₁₇ alkyl group, with bromomaleimde, IV-6, and IV-8 to produce Compounds B-15, B-17, and B-19, respectively. FIG. 6C shows the functionalization of Compound B-12, which is an embodiment of Formula B-11A, wherein the PEG moiety is PEG-2000, and R¹ is straight chain C₁₇ alkyl group, with bromomaleimde, IV-6, and IV-8 to produce Compounds B-16, B-18, and B-20, respectively.

FIGS. 7A-B depict a synthetic scheme for embodiments of symmetric di-ester PEG-lipids having a glycerol scaffold (S3) in which the PEG moiety terminates in a methoxyl group. FIG. 7A depicts a synthetic scheme for symmetric di-ester PEG-lipids of Formula B-21A. Specifically, FIG. 7B depicts synthesis of Compounds B-21 and B-22, which are embodiments of Formula B-21A, wherein the PEG moiety is methoxy-PEG-2000, and R¹ is branched and straight-chain C₁₇ alkyl groups, respectively.

FIGS. 8A-C depict a synthetic scheme for embodiments of symmetric di-ester PEG-lipids having a glycerol scaffold (S3) in which the PEG moiety terminates with a 4-methoxybenzyloxyl group (Formula VI-7A, and Compounds VI-7 and VI-8), their conversion to the related alcohols (Formula B-23A, and Compounds B-23 and B-24), and further functionalizations to functionalized PEG-lipids. FIG. 8A shows a synthetic scheme of PEG-lipids of Formula B-23A which are converted from PEG-lipids of Formula VI-7A by removal of the terminal 4-methoxybenzyloxyl group of the PEG moiety, and further functionalizations of PEG-lipids of Formula B-23A with bromomaleimde, IV-6, and IV-8 to provide functionalized PEG-lipids of Formulas B-25A, B-27A, and B-29A, respectively. Specifically, FIG. 8B depicts the synthesis of Compounds VI-7 and B-23, which are respectively embodiments of Formulas VI-7A and B-23A, wherein the PEG moiety is 4-methoxybenzyloxy-PEG-2000, and R¹ is branched C₁₇ alkyl group, and further functionalization of Compound B-23 with bromomaleimde, IV-6, and IV-8 to produce Compounds B-25, B-27, and B-29, respectively. FIG. 8C depicts the synthesis of Compounds VI-8 and B-24, which are respectively embodiments of Formulas VI-7A and B-23A, wherein the PEG moiety is 4-methoxybenzyloxy-PEG-2000, and R¹ is straight-chain C₁₇ alkyl group, and further functionalization of Compound B-24 with bromomaleimde, IV-6, and IV-8 to produce Compounds B-26, B-28, and B-30, respectively.

FIGS. 9A-F depict a synthetic scheme for embodiments of asymmetric di-ester PEG-lipids having a glycerol scaffold (S4). FIG. 9A shows a synthetic scheme for an asymmetric di-ester PEG-lipid of Formula B-31A wherein the PEG moiety has a terminal methoxyl group. FIG. 9B shows the synthesis of PEG derivatives of Formula VII-6 which has a PEG moiety having a terminal 4-methoxybenzyloxyl group at the one end and the other end attached to an asymmetric glycerol scaffold. FIG. 9C shows the synthesis of asymmetric di-ester PEG-lipids of Formula B-34A which are converted from PEG-lipids of Formula B-32A by removal of the terminal 4-methoxybenzyloxyl group of the PEG moiety. Specifically, FIG. 9D shows the synthesis of Compound B-31, which is an embodiment of Formula B-31A, wherein the PEG moiety is methoxy-PEG-2000, and R¹ is branched C₁₇ alkyl group. FIG. 9E depicts synthesis of Compound VII-6, an embodiment of the PEG derivatives of Formula VII-6, wherein the PEG moiety is 4-methoxybenzyloxy-PEG 2000. FIG. 9F shows the synthesis of Compounds B-33 and B-35, which are embodiments of Formulas B-32A and B-34A, wherein the PEG moiety is 4-methoxybenzyloxy-PEG 2000 and PEG-2000, respectively, and R¹ is branched C₁₇ alkyl group; and Compounds B-32 and B-34, which are embodiments of Formulas B-32A and B-34A, wherein the PEG moiety is 4-methoxybenzyloxy-PEG 2000 and PEG-2000, respectively, and R¹ is straight-chain C₁₇ alkyl group.

FIGS. 10A-C depict the functionalization of PEG-lipids of Formula B-34A with bromomaleimde, IV-6, and IV-8 to provide functionalized PEG-lipids of Formulas B-36A, B-38A, and B-40A, respectively. Specifically, FIG. 10B depicts the functionalization of Compound B-34, an embodiment of Formula B-34A wherein the PEG moiety is PEG-2000, and R¹ is straight-chain C₁₇ alkyl group, with bromomaleimde, IV-6, and IV-8 to provide Compounds B-36, B-38, and B-40, respectively. FIG. 10C depicts the functionalization of Compound B-35, an embodiment of Formula B-34A wherein the PEG moiety is PEG-2000, and R¹ is branched C₁₇ alkyl group, with bromomaleimde, IV-6, and IV-8 to provide Compounds B-37, B-39, and B-41, respectively.

FIGS. 11A-B depict a synthetic scheme for embodiments of symmetric tri-ester PEG-lipids built on a scaffold of formula S1 wherein the PEG moiety terminates in methoxyl, benzyloxyl, or 4-methoxybenzyloxyl group. FIG. 11A depicts a synthetic scheme for PEG-lipids of Formulas B-42A, B-44A, and B-46A. Specifically, FIG. 11B depicts synthesis of Compounds B-42, B-44, and B-46, which are embodiments of Formulas B-42A, B-44A, and B-46A, respectively, wherein the PEG moiety is PEG-2000 derivative and R¹ is straight-chain C₁₇ alkyl group, as well as synthesis of Compounds B-43, B-45, and B-47, which are embodiments of Formulas B-42A, B-44A, and B-46A, respectively, wherein the PEG moiety is PEG-2000 derivative and R¹ is branched C₁₇ alkyl group.

FIG. 12 depict a synthesis scheme of preparation of embodiments of functionalized PEG-lipids having bromomaleimide.

FIG. 13 summarize certain embodiments of PEG-lipids disclosed herein.

FIGS. 14A-C depict the viability (14A), frequency of transfection (14B), and level of expression as geometric mean fluorescence intensity (gMFI) of the transfected cells (14C) for HEK293F cells transfected with mCherry mRNA encapsulated in LNP in which the PEG-lipid was one of Compounds VI-7, VI-8, B46, B47, or DMG-PEG2000.

FIG. 15 depicts the results of transfection of mouse splenic T cells with mCherry mRNA encapsulated in LNP conjugated to an anti-CD5 mAb as a plot of transfection frequency versus geometric mean fluorescence intensity. The antibody was conjugated to Compounds B-3, B-25, or DSPE-PEG-maleimide. Compounds B-3 and B-25 comprise a bromomaleimide moiety used for the conjugation.

### DETAILED DESCRIPTION

The instant disclosure provides PEG-lipids and functionalized PEG-lipids, methods for synthesizing them, as well as intermediates useful in synthesis of these lipids and methods of synthesizing the intermediates. The instant disclosure further provides PEG-lipids as a component of lipid nanoparticles (LNPs), which LNPs can be used for the delivery of nucleic acids into cells in vivo or ex vivo. LNP compositions are also disclosed herein, including LNPs comprising a functionalized PEG-lipid to enable conjugation of a binding moiety to generate targeted LNPs (tLNPs), that is LNPs containing a binding moiety, conjugated to the functionalized PEG-lipid, that directs the tLNP to a desired tissue or cell type. Also disclosed herein are methods of delivering a nucleic acid into a cell comprising contacting the cell with a LNP or tLNP of this disclosure.

Prior to setting forth this disclosure in more detail, it may be helpful to provide abbreviations and definitions of certain terms to be used herein. Additional definitions are set forth throughout this disclosure.

### Abbreviations

### Abbreviations Used Herein Include:

BOC—tert-Butyloxycarbonyl
cLogD—calculated LogD
c-pKa—calculated pKa
DIAD—Diisopropyl azodicarboxylate
DMF—Dimethylformamide
DMAP—4-Dimethylaminopyridine
EDC-HCl—1-Ethyl-3-(3′-dimethylaminopropyl)carbodiimide·HCl
Et3N—Triethylamine
HOAc—acetic acid
MeOH—Methanol
Me₃Si—Trimethylsilyl
Pd/C—Palladium on carbon
PEG—Polyethylene glycol
PEG—DMG-Polyethylene glycol-dimyristoyl glycerol
Ph₃P—Triphenylphosphine
KOtBu—Potassium tert-butoxide
P-TsOH—p-Toluenesulfonic acid
PPTs—Pyridinium P-toluenesulfonate
TFA—Trifluoroacetic acid
THF—Tetrahydrofuran

### Definitions

As used in the specification and claims, the singular form “a,” “an,” and “the” includes plural references unless the context clearly dictates otherwise. It should be understood that the terms “a” and “an” as used herein refer to “one or more” of the enumerated components.

The use of the alternative (e.g., “or”) should be understood to mean either one, both, or any combination thereof of the alternatives.

The term “about” or “≈” as used herein in the context of a number refers to a range centered on that number and spanning 15% less than that number and 15% more than that number. The term “about” used in the context of a range refers to an extended range spanning 15% less than that the lowest number listed in the range and 15% more than the greatest number listed in the range.

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

Unless the context requires otherwise, throughout the present specification and claims, the word “comprise” and variations thereof, such as, “comprises” and “comprising” are to be construed in an open, inclusive sense, that is, as “including, but not limited to.” As used herein, the terms “include” and “comprise” are used synonymously.

The phrase “at least one of” when followed by a list of items or elements refers to an open-ended set of one or more of the elements in the list, which may, but does not necessarily, include more than one of the elements.

“Derivative,” as used herein, refers to a chemically or biologically modified version of a parent compound that is structurally similar to a parent compound and (actually or theoretically) derivable from that parent compound. Generally, a “derivative” differs from an “analogue” in that a parent compound may be the starting material to generate a “derivative,” whereas the parent compound may not necessarily be used as the starting material to generate an “analogue.” A derivative may have chemical or physical properties that are different from those of the parent compound. For example, a derivative may be more hydrophilic or hydrophobic, or it may have altered reactivity as compared to the parent compound.

Alkyl refers to a saturated hydrocarbon moiety, that is an alkane lacking one hydrogen leaving a bond that connects to another portion of an organic molecule. In some embodiments, hydrogens are unsubstituted. In other embodiments, one or more hydrogens of the alkyl group may be substituted with the same or different substituents.

Alkenyl refers to a hydrocarbon moiety with one or more carbon-carbon double bonds but that is otherwise saturated. In some embodiments, hydrogens are unsubstituted. In other embodiments, one or more hydrogens of the alkenyl group may be substituted with the same or different substituents.

Alkynyl refers to a hydrocarbon moiety with one or more carbon-carbon triple bonds but that is otherwise saturated. In some embodiments, hydrogens are unsubstituted. In other embodiments, one or more hydrogens of the alkynyl group may be substituted with the same or different substituents.

Alkynylamide refers to an amide comprising a carbon-carbon triple bond immediately adjacent to the carbonyl group of the amide. However, in certain embodiments, the amide is a secondary amide.

Alkynylimide refers to an imide comprising a carbon-carbon triple bond immediately adjacent to at least one of the two carbonyl groups of the imide. However, in certain embodiments, the second carbonyl group does not have an immediately adjacent carbon-carbon triple bond.

Alkynoic refers to a carboxylic acid moiety comprising one or more carbon-carbon triple bonds. In some embodiments, hydrogens are unsubstituted. In other embodiments, one or more hydrogens of the alkynoic group may be substituted with the same or different substituents.

Amide refers to a carboxylic acid derivative comprising a carbonyl group of a carboxylic acid bonded to an amine moiety.

Aryl refers to an aromatic or heteroaromatic ring lacking one hydrogen leaving a bond that connects to another portion of an organic molecule. Examples of aryl include, without limitation, phenyl, naphthalenyl, pyridine, pyrimidine, pyrazine, pyrrole, furan, thiophene, imidazole, thiazole, oxazole, and the like.

Aryl-alkyl refers to a moiety comprising one or more aryl rings and one or more alkyl moieties. The position of the one or more aryl rings can vary within the alkyl portion of the moiety. For example, the one or more aryl rings may be at an end of the one or more alkyl moieties, be fused into the carbon chain of the one or more alkyl moieties, or substitute one or more hydrogens of one or more alkyl moieties; and the one or more alkyl moieties may substitute one or more hydrogens of the one or more aryl rings. In some embodiments, there is a single ring; while in other embodiments, that are multiple rings.

Branched alkyl is a saturated alkyl moiety wherein the alkyl group is not a straight chain. Alkyl portions such as methyl, ethyl, propyl, butyl, and the like, can be appended to variable positions of the main alkyl chain. In some embodiments, there is a single branch; while in other embodiments, there are multiple branches.

Branched alkenyl refers to an alkenyl group comprising at least one branch off the main chain which may be formed by substituting one or more hydrogens of the main chain with the same or different alkyl groups, e.g., without limitation, methyl, ethyl, propyl, butyl, and the like. In some embodiments, a branched alkenyl is a single branch structure, while in other embodiments, a branched alkenyl may have multiple branches.

Straight chain alkyl is a non-branched, non-cyclic version of the alkyl moiety described above.

Straight chain alkenyl is a non-branched, non-cyclic version of the alkenyl moiety described above.

Cycloalkyl refers to a moiety which is a cycloalkyl ring of 3-12 carbons. In some embodiments, a cycloalkyl is a single ring structure; while in other embodiments, a cycloalkyl may have multiple rings.

Cycloalkyl-alkyl refers to a moiety which contains one or more cycloalkyl rings of 3-12 carbons, and one or more alkyl moieties. The position of the cycloalkyl ring can vary within the alkyl portion of the moiety. For example, the one or more cycloalkyl rings may be at an end of the one or more alkyl moieties, be fused into the carbon chain of the one or more alkyl moieties, or substitutes one or more hydrogens of one or more alkyl moieties; and the one or more alkyl moieties may substitute one or more hydrogens of the one or more cycloalkyl rings. In some embodiments, the cycloalkyl ring is a single ring structure; while in other embodiments, a cycloalkyl-alkyl may have multiple rings.

Ester refers to a carboxylic acid derivative comprising a carbonyl group bond to an alkyloxy group to form an ester bond —C(═O)—O—.

Ether refers to an oxygen atom attached to 2 carbon-based moieties that are the same or different.

Fatty acid refers to a carboxylic acid comprising a saturated or unsaturated carbon chain, unbranched or branched, uninterrupted and unsubstituted by heteroatoms. In certain embodiments, a fatty acid generally has 10 to 30 carbon atoms. In certain embodiments, a fatty acid has 14 or more carbon atoms.

Head group refers to the hydrophilic or polar portion of a lipid.

Imide refers to a moiety comprising a nitrogen bond to two carbonyl groups.

Sterol refers to a subgroup of steroids that contain at least one hydroxyl (OH) group. Examples of sterols include, without limitation, cholesterol, ergosterol, $-sitosterol, stigmasterol, stigmastanol, 20-hydroxycholesterol, 22-hydroxycholesterol, and the like.

### PEG-Lipids

Polyethylene glycol-lipids (PEG-lipids) are useful as a component of lipid nanoparticles for the delivery of nucleic acids, including DNA, mRNA, guide RNA, or siRNA into cells both to prevent aggregation of LNP, to prevent potentially undesired binding to transport proteins or cell surface receptors, to provide a hydrophilic surface for the LNP, and as a substrate for the conjugation of a binding moiety that can serve to target the LNP to a desired tissue or cell type. However, the commonly used PEG-lipids, derived from diacylglycerols and phospholipids, have several potentially problematic features.

In the common PEG-lipids, glycerol serves as the linkage between two fatty acid tails and a PEG moiety. Originally designed to resemble phospholipids, the PEG is attached at one of glycerol's primary hydroxyls with fatty acids attached at the other primary hydroxyl and at the secondary hydroxyl, generating a molecule that is chiral. It is known that LNP incorporating optically pure (homochiral) PEG-lipids have superior performance in releasing their nucleic acid cargo into the cytoplasm (endosomal escape), but racemic preparations are less expensive to obtain, and the optically pure species are subject to racemization through chain migration.

Novel PEG-Lipids Comprising Symmetrical Scaffolds that are not Glycerol-Based (S1 and S2) or Glycerol-Based (S3), and Novel PEG-Lipids Comprising Asymmetric Glycerol-Based Scaffold (S4)

In certain aspects, the instant disclosure provides PEG-lipids comprising a symmetrical scaffold of formula S1 or S2 that is not glycerol-based. PEG-lipids comprising scaffold of formula S1 or S2 are symmetrical, obviating issues related to chirality at least of the scaffold. Alternatively, a glycerol-based symmetrical PEG-lipid can be obtained by attaching the PEG moiety at the secondary hydroxyl and the same fatty acid at each of the two primary hydroxyls (see, e.g., scaffold of formula S3).

In certain embodiments of the PEG-lipids disclosed herein, the PEG moiety is connected to the scaffold by an ether bond or an ester bond. In embodiments disclosed herein, the fatty acid moieties are connected to the scaffold by biodegradable ester bonds to improve biodegradability.

The most commonly used PEG-lipid may be PEG-dimyristoyl glycerol (PEG-DMG). However, PEG-DMG is shed from LNP which is particularly of concern when a binding moiety is conjugated, as the targeting function mediated by the binding moiety will be lost from LNP. Accordingly, certain embodiments provide PEG lipids comprising C₁₆-C₂₀ straight-chain fatty acid esters or C₁₄-C₂₀ branched-chain fatty acid esters which in certain instances are conjugated to a binding moiety.

In certain aspects, the present disclosure provides a symmetrical tri-ester PEG-lipid in which an esterified PEG moiety is attached to a central position on a scaffold and two identical fatty acids are esterified to two end positions on the scaffold. In some embodiments, the fatty acid esters are not positioned immediately adjacent to the branch point, for example they are in a β position relative to the branch point. In some embodiments, the esterified PEG moiety is not positioned immediately adjacent to the branch point, for example it is in a β or γ position relative to the branch point. In some embodiments, the scaffold has the structure of Formula S1

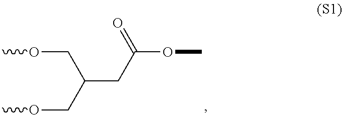

where [symbol] represents the points of ester connection with a fatty acid and [symbol] represents the point of ester formation with the PEG moiety. In some embodiments, the fatty acid esters are C₁₄-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the PEG moiety is functionalized and the fatty acid esters are C₁₆-C₂₀ straight-chain alkyl fatty acids, e.g., C₁₆, C₁₇, C₁₈, C₁₉, or C₂₀ straight-chain alkyl fatty acids. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. In some embodiments, the branch is at the 3rd, 4th, 5th, 6th, or 7th carbon position starting from the carbonyl of the fatty acid ester.

In some embodiments, the symmetrical tri-ester PEG-lipid disclosed herein with a scaffold of formula S1 has a structure of Formula PL-1

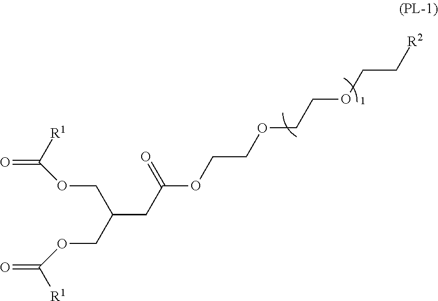

wherein R¹ is a C₁₃-C₁₉ alkyl which is a straight-chain or symmetric branched-chain, examples of symmetric branched-chain include, without limitation, —(CH₂)—CH(R⁴)₂, —(CH₂)₂—CH(R⁵)₂, —(CH₂)₃—CH(R⁶)₂, —(CH₂)₄—CH(R⁷)₂, and —(CH₂)₅—CH(R⁸)₂, wherein R⁴, R⁵, R⁶, R⁷, and R⁸ are defined the same as in Formulas BFA-1, BFA-2, BFA-3, BFA-4, and BFA-5, respectively, R² is —OH, —OCH₃, —O-benzyl, 4-methoxybenzyloxyl, maleimide, azide, alkynyl, DBCO, bromomaleimide

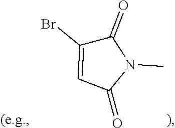

bromomaleimide amide (e.g., 1-bomomaleimido-acetic acid amide), alkynylimide, or alkynylamide, and n≈10 to 115.

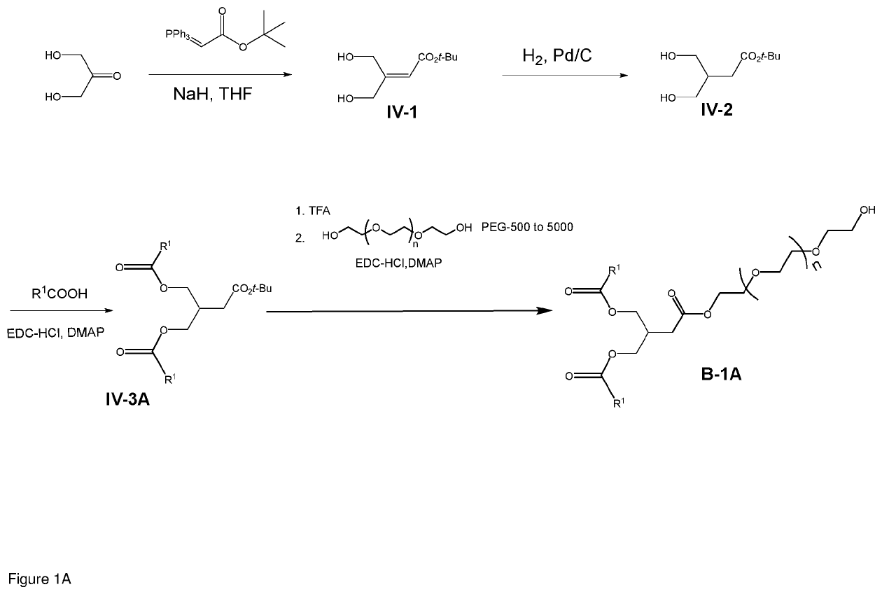

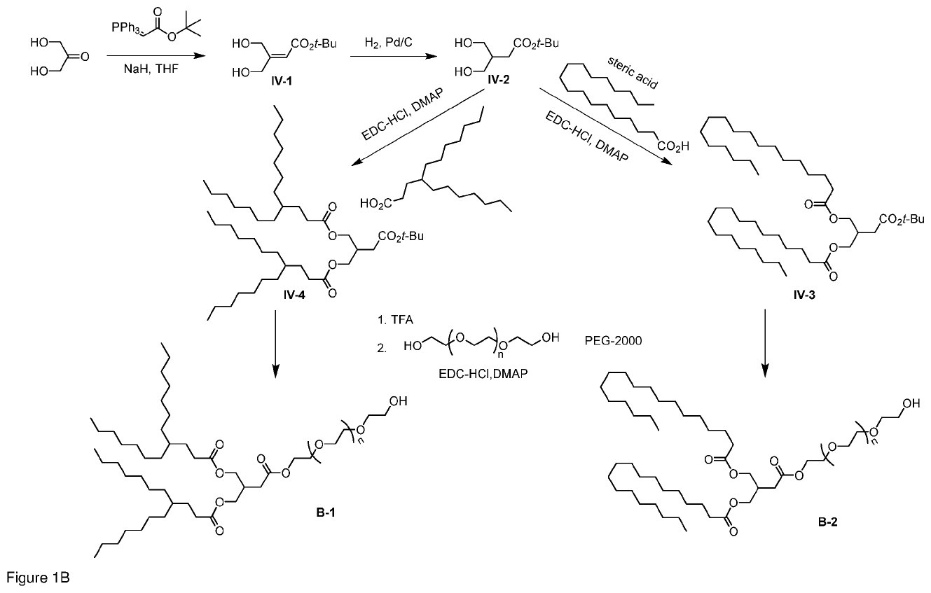

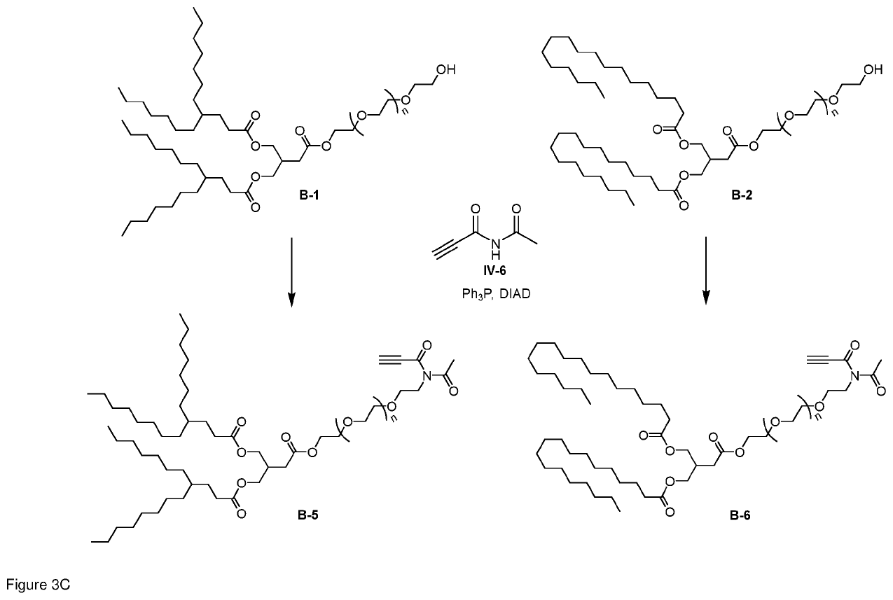

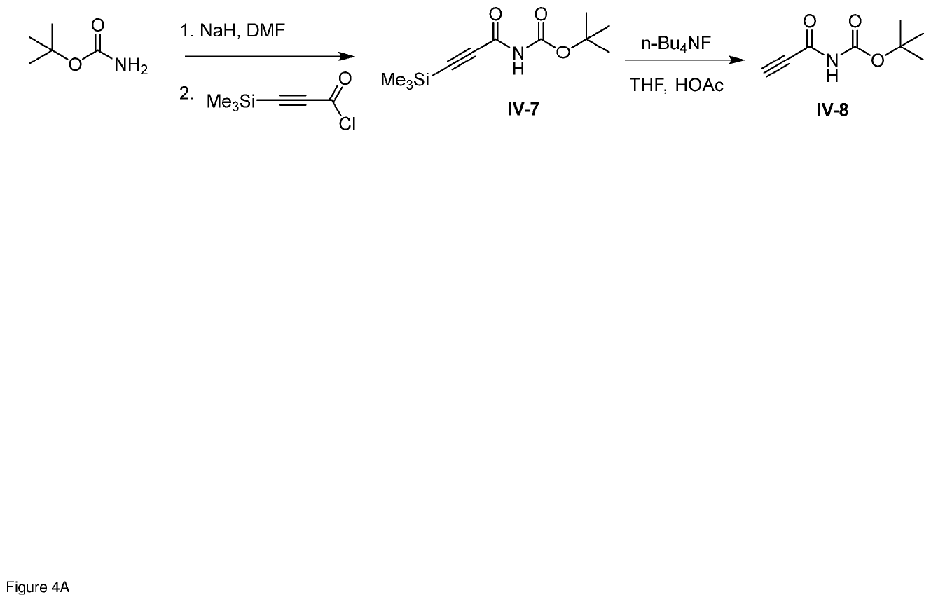

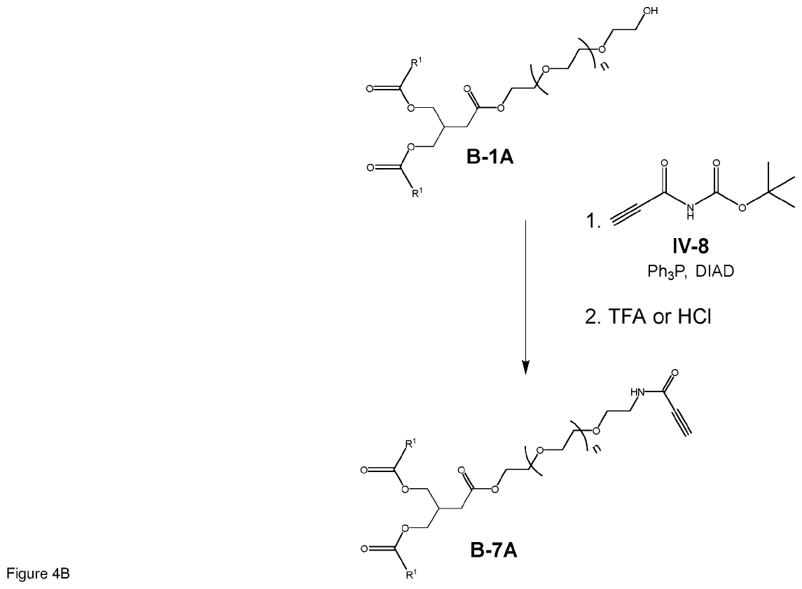

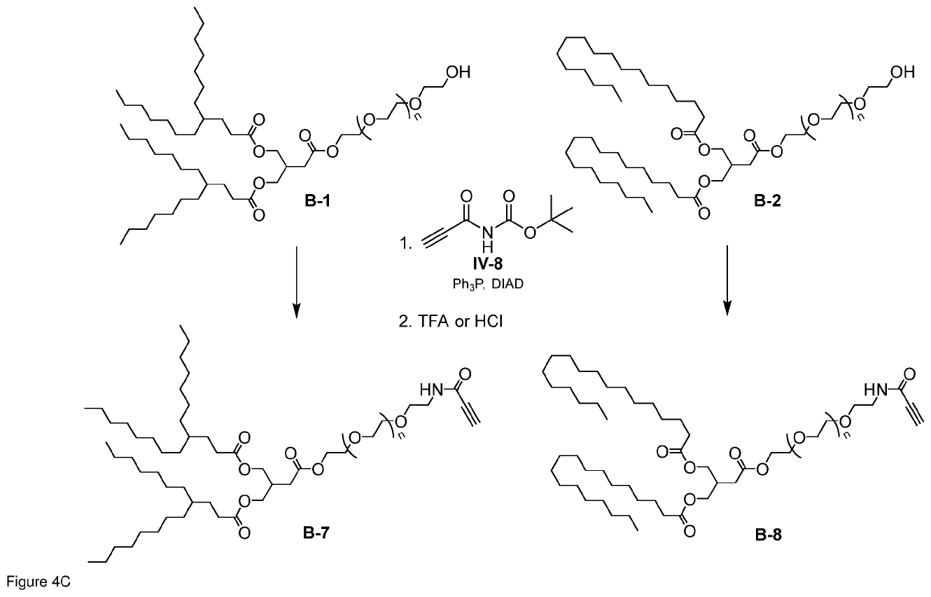

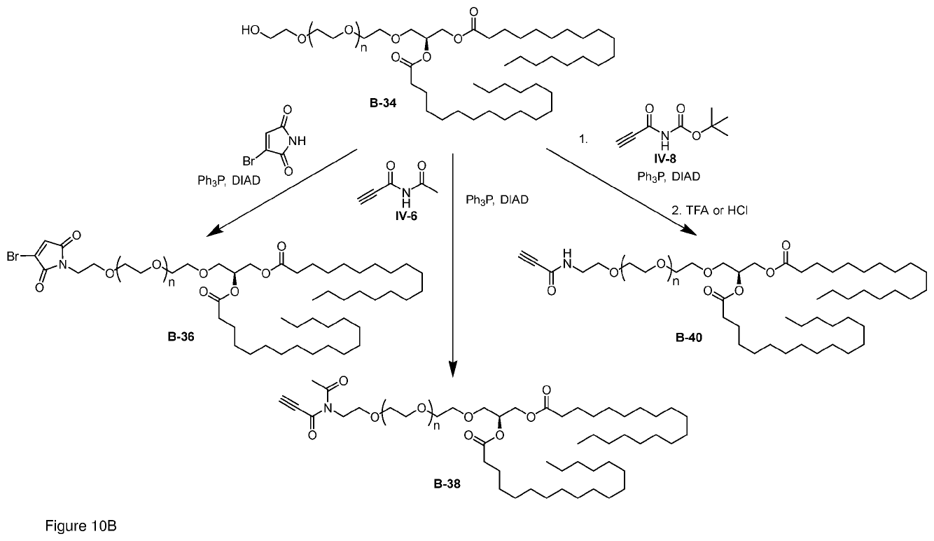

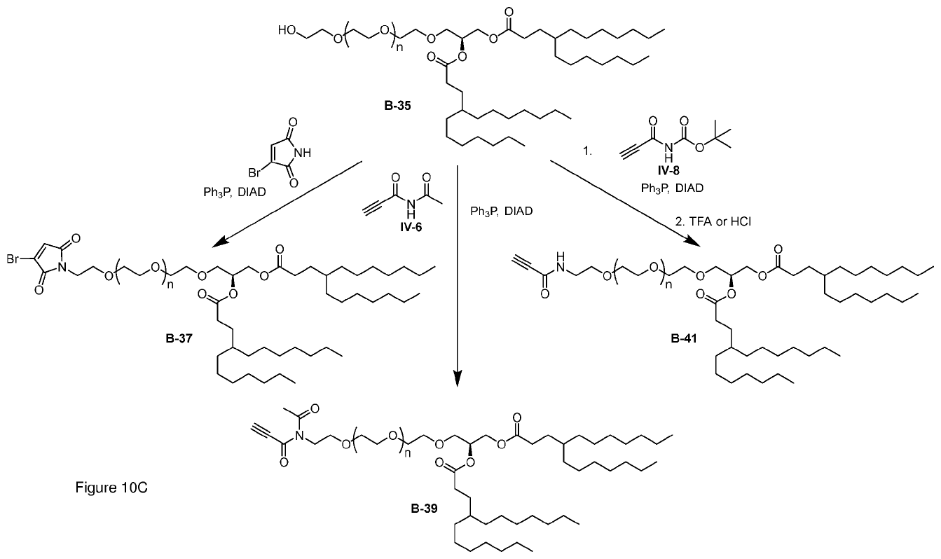

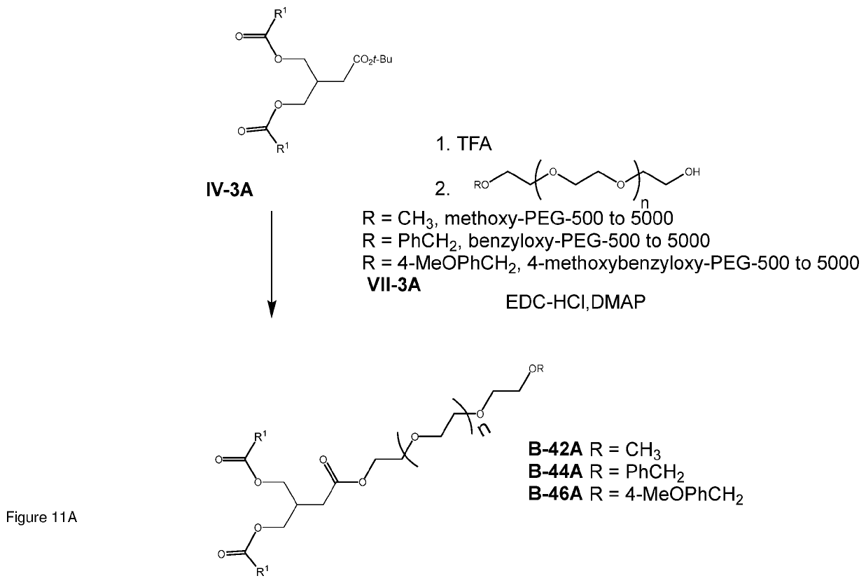

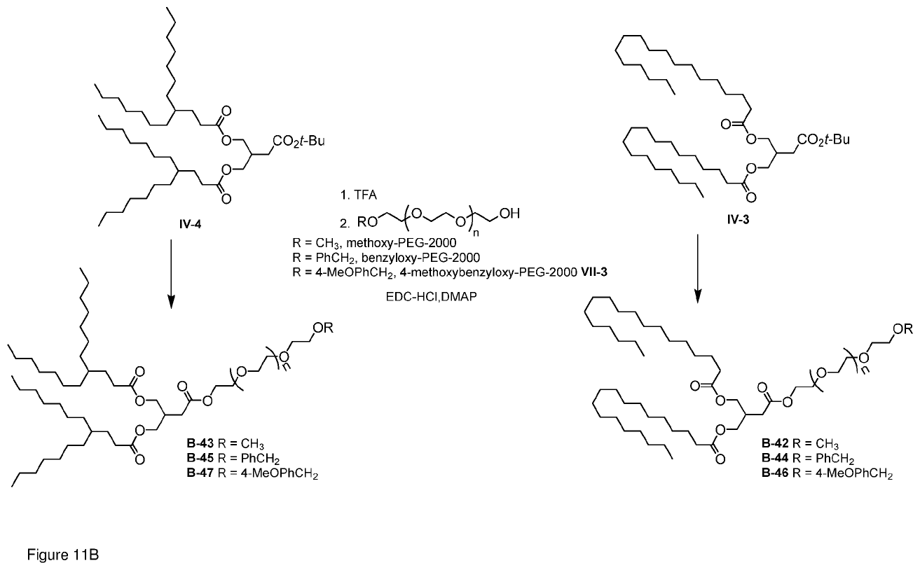

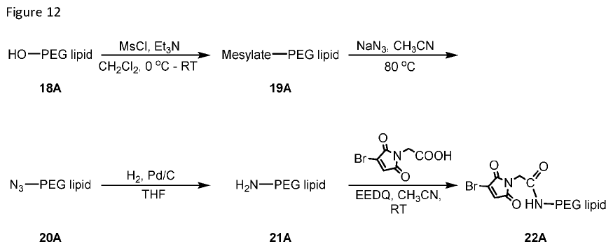

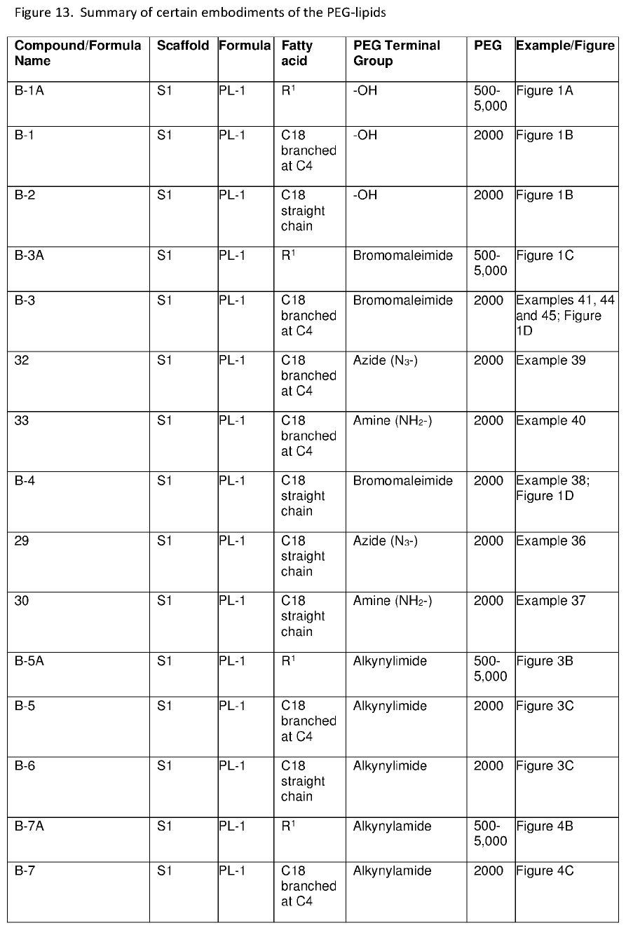

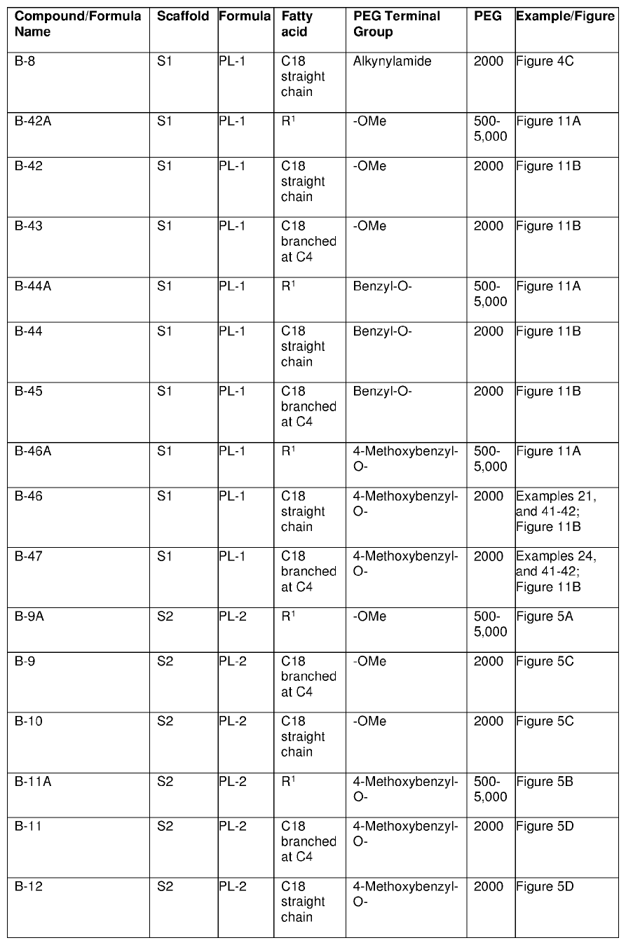

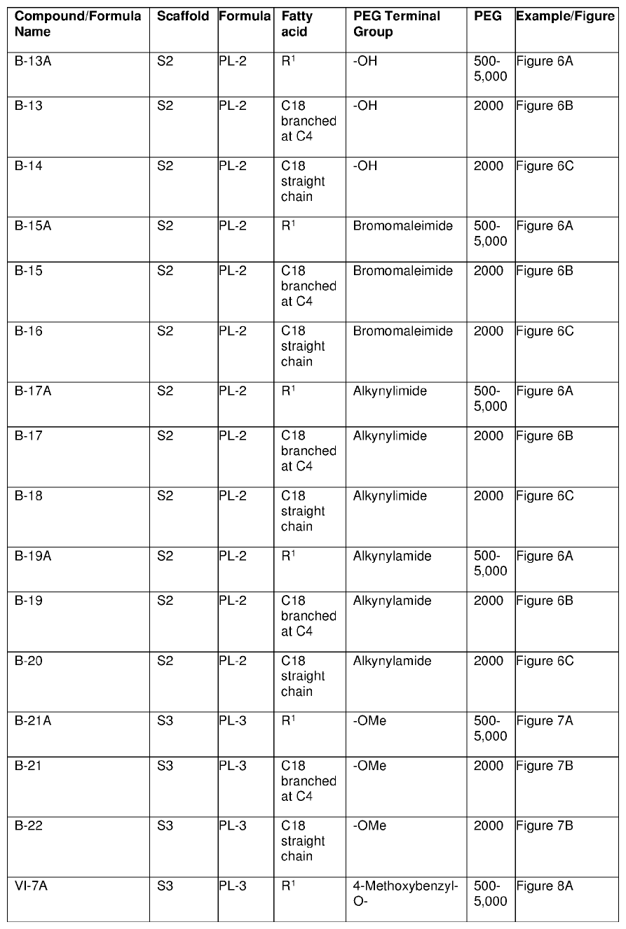

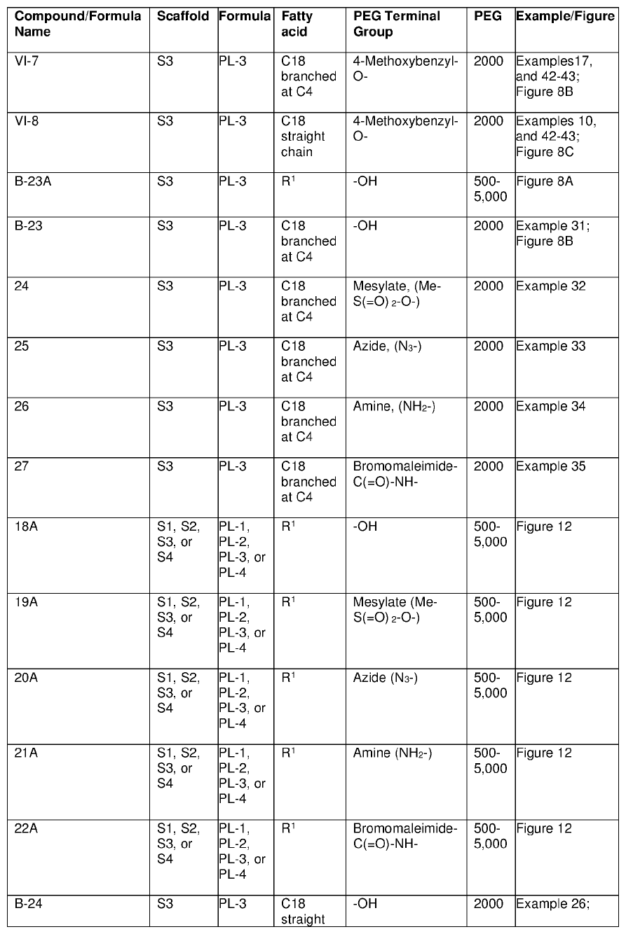

In some embodiments, the symmetrical tri-ester PEG-lipid with a scaffold of formula PL-1 is functionalized with a maleimide, azide, alkyne, bromomaleimide, bromomaleimide amide, alkynylimide, or alkynylamide. In some embodiments, the PEG moiety is functionalized and the fatty acid esters are C₁₆-C₂₀ straight-chain alkyl fatty acids such as C₁₆, C₁₇, C₁₈, C₁₉, or C₂₀ straight-chain alkyl fatty acids. In some embodiments having a C₁₄-C₂₀ symmetric branched-chain alkyl fatty acid ester, for example, the branched-chain alkyl fatty acid is C₁₄, C₁₅, C₁₆, C₁₇, C₁₈, C₁₉, or C₂₀. In some embodiments, the branch is at the 3rd, 4th, 5th, 6th, or 7th carbon position starting from the carbonyl of the fatty acid ester. In some embodiments, the symmetrical tri-ester PEG-lipids with a scaffold of formula S1 or a structure of Formula PL-1 have a structure of Formula B-1A (FIG. 1A), B-3A (FIG. 1C), B-5A (FIG. 3B), B-7A (FIG. 4B), B-42A (FIG. 11A), B-44A (FIG. 11A), B-46A (FIG. 11A), 18A (FIG. 12), 19A (FIG. 12), 20A (FIG. 12), 21A (FIG. 12), or 22A (FIG. 12). In some embodiments, the symmetrical tri-ester PEG-lipid with a scaffold of formula S1 or a structure of Formula PL-1 is Compound B-1 (FIG. 1B), B-3 (FIG. 1D, Examples 41, 44 and 45), 32 (Example 39), 33 (Example 40), B-5 (FIG. 3C), B-7 (FIG. 4C), B-43 (FIG. 11B), B-45 (FIG. 11B), or B-47 (FIG. 11B, Examples 25, 42 and 43). In some embodiments, the PEG-lipid with scaffold of formula S1 or a structure of Formula PL-1 is Compound B-2 (FIG. 1B), B-4 (FIG. 1D, Example 38), 29 (Example 36), 30 (Example 37), B-6 (FIG. 3C), B-8 (FIG. 4C), B-42 (FIG. 11B), B-44 (FIG. 11B), or B-46 (FIG. 11B, Examples 22, 42 and 43).

In other aspects, the present disclosure provides a symmetrical di-ester PEG-lipid in which a PEG-moiety is attached to a central position on a scaffold by an ether linkage and two identical fatty acids are esterified to two end positions on the scaffold. In some embodiments, the fatty acid esters are not positioned immediately adjacent to the branch point, for example they are in a β position relative to the branch point. In some embodiments, the PEG moiety is not positioned immediately adjacent to the branch point, for example it is in a β or γ position relative to the branch point.

In some embodiments, the scaffold has the structure of Formula S2

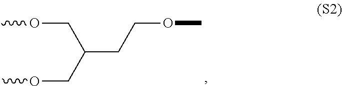

where [symbol] represents the points of esterification of fatty acids and [symbol] represents the ether linkage to the PEG moiety. In some embodiments, the fatty acid esters are C₁₄-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the PEG moiety is functionalized and the fatty acid esters are C₁₆-C₂₀ straight-chain alkyl fatty acids, e.g., C₁₆, C₁₇, C₁₈, C₁₉, or C₂₀ straight-chain alkyl fatty acids. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. In some embodiments, the branch is at the 3rd, 4th, 5th, 6th, or 7th carbon position starting from the carbonyl of the fatty acid ester.

In some embodiments, the symmetrical di-ester PEG-lipid with a glycerol scaffold has a structure of Formula PL-2

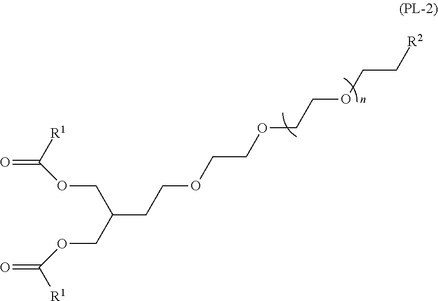

wherein R¹, R² and n are defined the same as R¹, R² and n with respect to Formula PL-1 set forth supra.

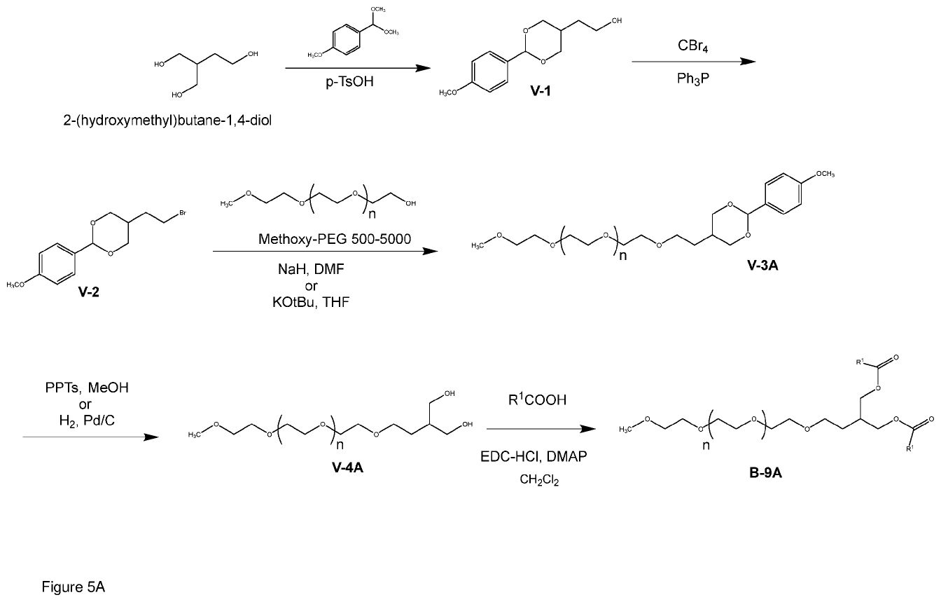

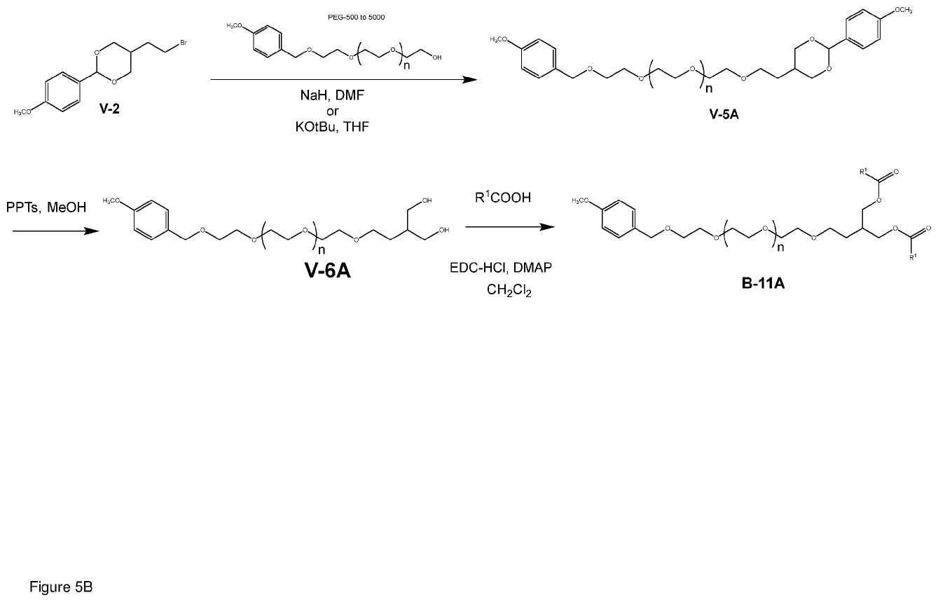

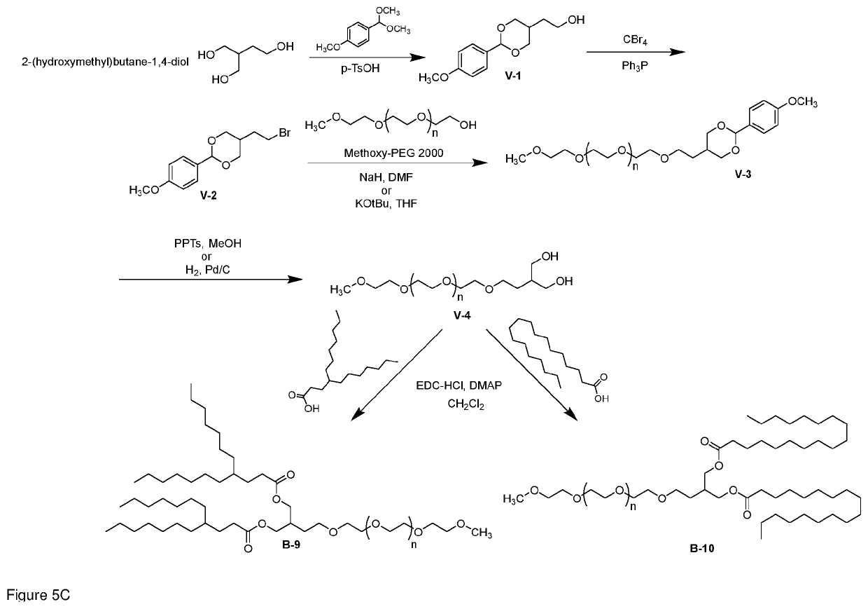

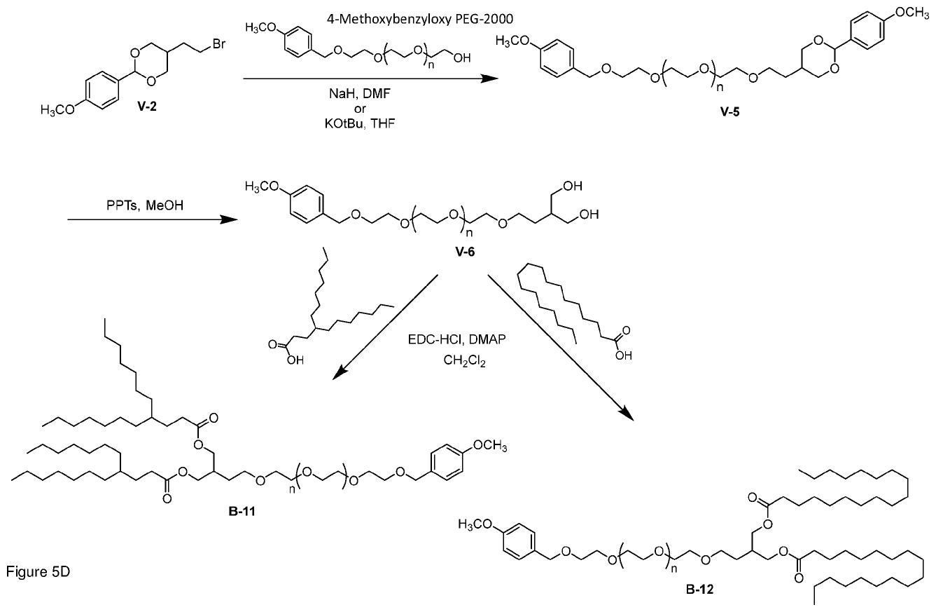

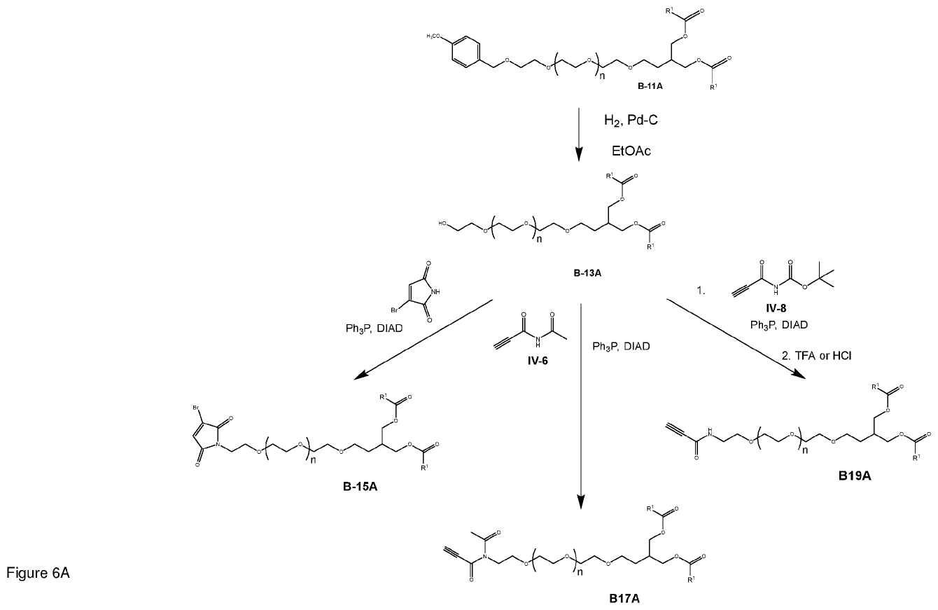

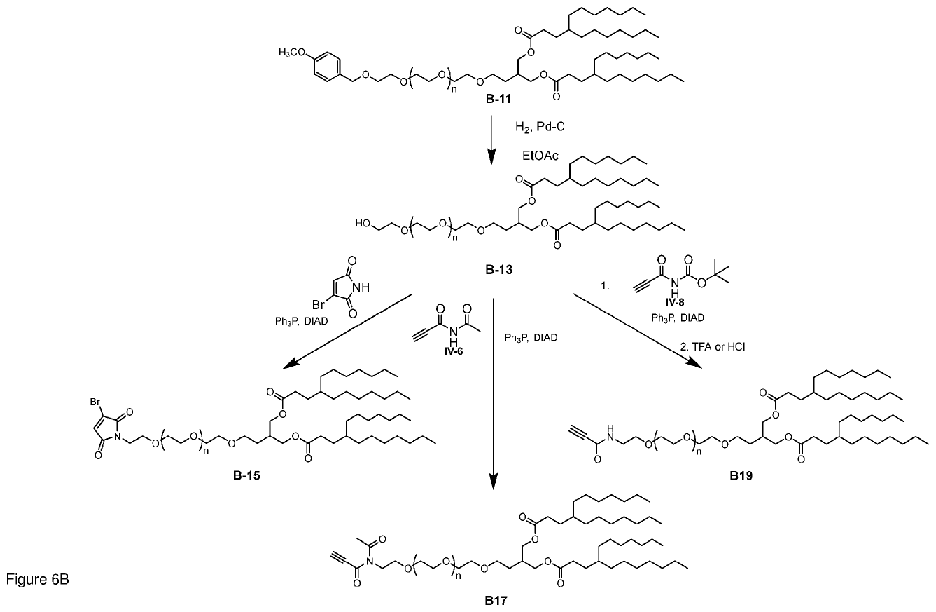

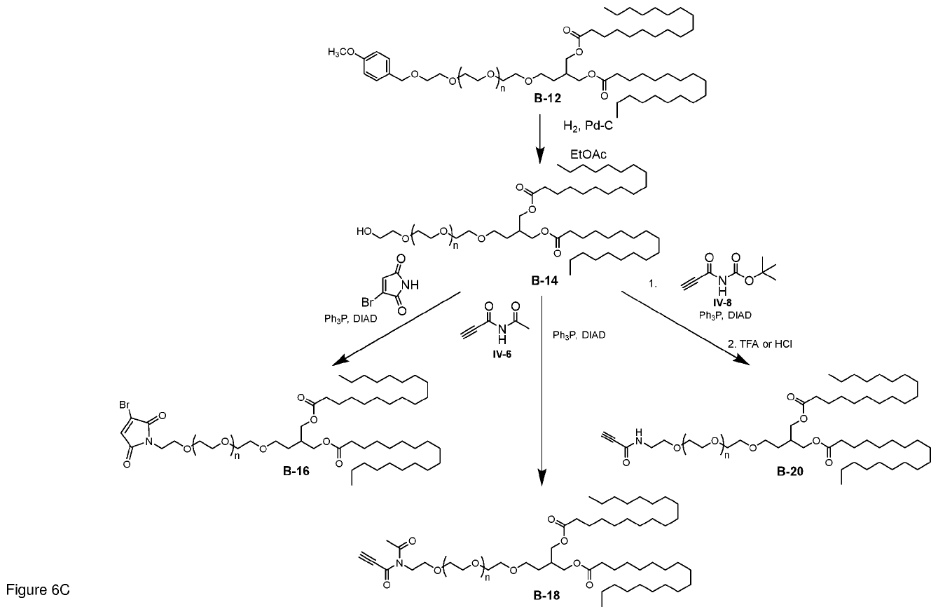

In some embodiments, the symmetrical di-ester PEG-lipid with a structure of Formula PL-2 is functionalized with a maleimide, azide, alkyne, DBCO, bromo-maleimide, alkynylimide, or alkynylamide. In some embodiments, the PEG moiety is functionalized and the fatty acid esters are C₁₆-C₂₀ straight-chain alkyl fatty acids such as C₁₆, C₁₇, C₁₈, C₁₉, or C₂₀ straight-chain alkyl fatty acids. In some embodiments having a C₁₄-C₂₀ symmetric branched-chain alkyl fatty acid ester, for example, the branched-chain alkyl fatty acid is C₁₄, C₁₅, C₁₆, C₁₇, C₁₈, C₁₉, or C₂₀. In some embodiments, the branch is at the 3rd, 4th, 5th, 6th, or 7th carbon position starting from the carbonyl of the fatty acid ester. In some embodiments, the symmetrical di-ester PEG-lipids with a scaffold of formula S2 or a structure of Formula PL-2 have a structure of Formula B-9A (FIG. 5A), B-11 A (FIG. 5B), B-13A (FIG. 6A), B-15A (FIG. 6A), B-17A (FIG. 6A), B-19A (FIG. 6A), 18A (FIG. 12), 19A (FIG. 12), 20A (FIG. 12), 21A (FIG. 12), or 22A (FIG. 12). In some embodiments, the symmetric di-ester PEG-lipid with a scaffold of Formula S2 or a structure of Formula PL-2 is Compound B-9 (FIG. 5C), B-10 (FIG. 5C), B-11 (FIG. 5D), B-12 (FIG. 5D), B-13 (FIG. 6B), B-14 (FIG. 6C), B-15 (FIG. 6B), B-16 (FIG. 6C), B-17 (FIG. 6B), B-18 (FIG. 6C), B-19 (FIG. 6B), or B-20 (FIG. 6C).

In some embodiments, the scaffold of the symmetric di-ester PEG-lipid has the structure of Formula S3

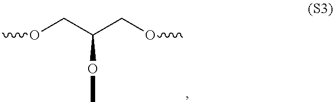

a glycerol scaffold, where [symbol] represents the points of esterification of the fatty acids and [symbol] represents the ether linkage to the PEG moiety. In some embodiments, the fatty acid esters are C₁₄-C₂₀ straight-chain alkyl fatty acids or any integer value or integer-bound range therein. For example, the straight-chain alkyl fatty acid is C₁₄, C₁₅, C₁₆, C₁₇, C₁₈, C₁₉, or C₂₀. In some embodiments, the PEG moiety is functionalized and the fatty acid esters are C₁₆-C₂₀ straight-chain alkyl fatty acids, e.g., C₁₆, C₁₇, C₁₈, C₁₉, or C₂₀ straight-chain alkyl fatty acids. In some embodiments, the fatty acid esters are C₁₄-C₂₀ symmetric branched-chain alkyl fatty acid esters or any integer value or integer bound range therein. For example, the branched-chain alkyl fatty acid is C₁₄, C₁₅, C₁₆, C₁₇, C₁₈, C₁₉, or C₂₀. In some embodiments, the branch is at the 3rd, 4th, 5th, 6th, or 7th carbon position starting from the carbonyl of the fatty acid ester.

In some embodiments, the symmetrical di-ester PEG-lipid with a glycerol scaffold has a structure of Formula PL-3

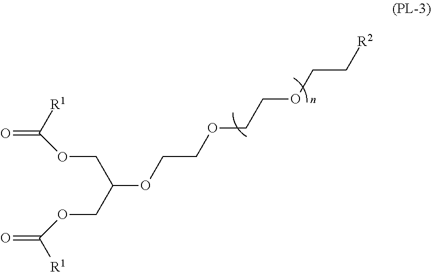

wherein R¹, R² and n are defined the same as R¹, R² and n with respect to Formula PL-1 set forth supra.

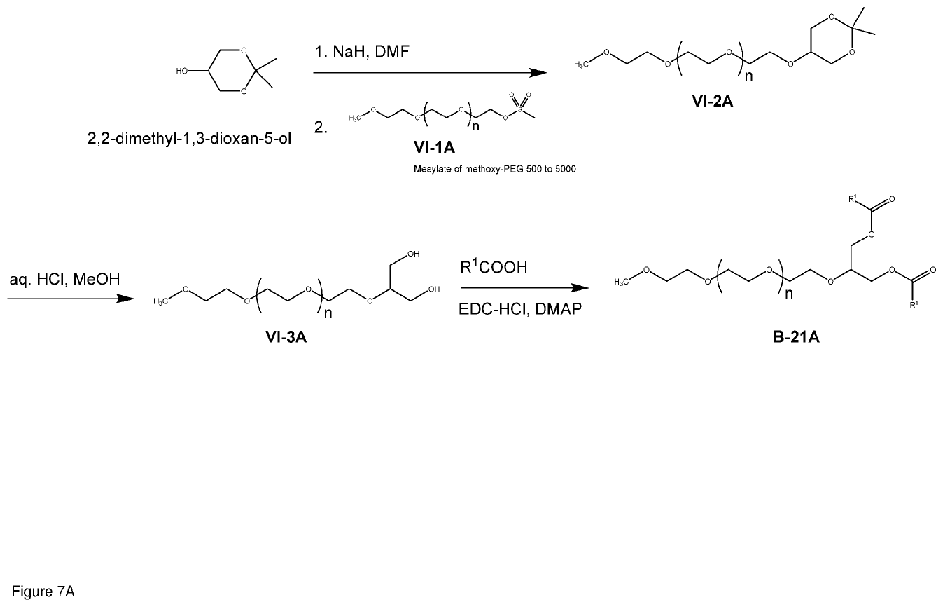

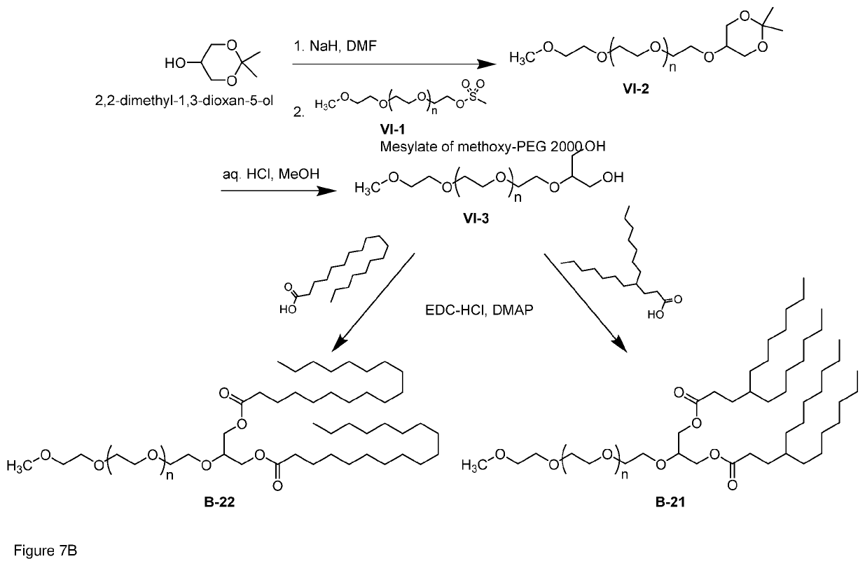

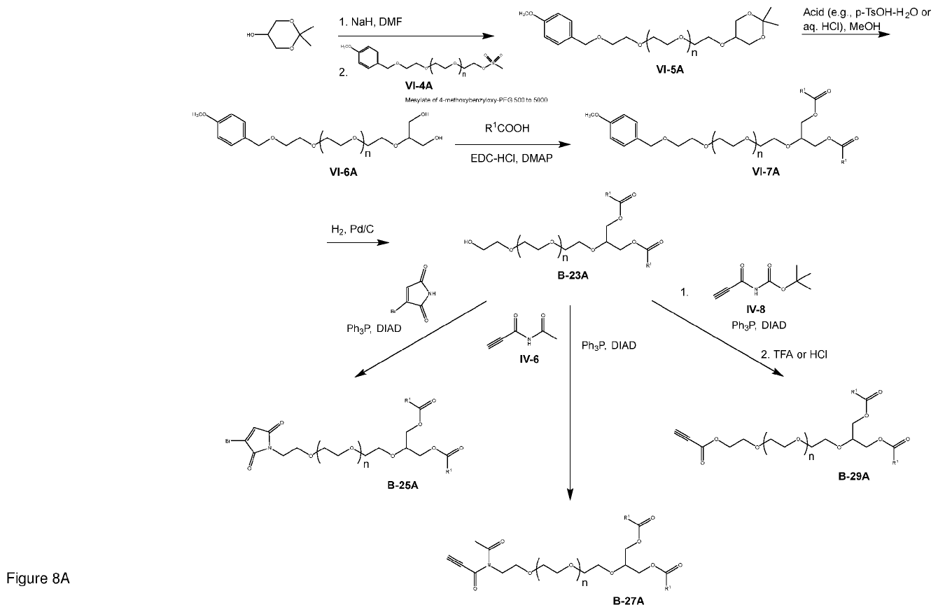

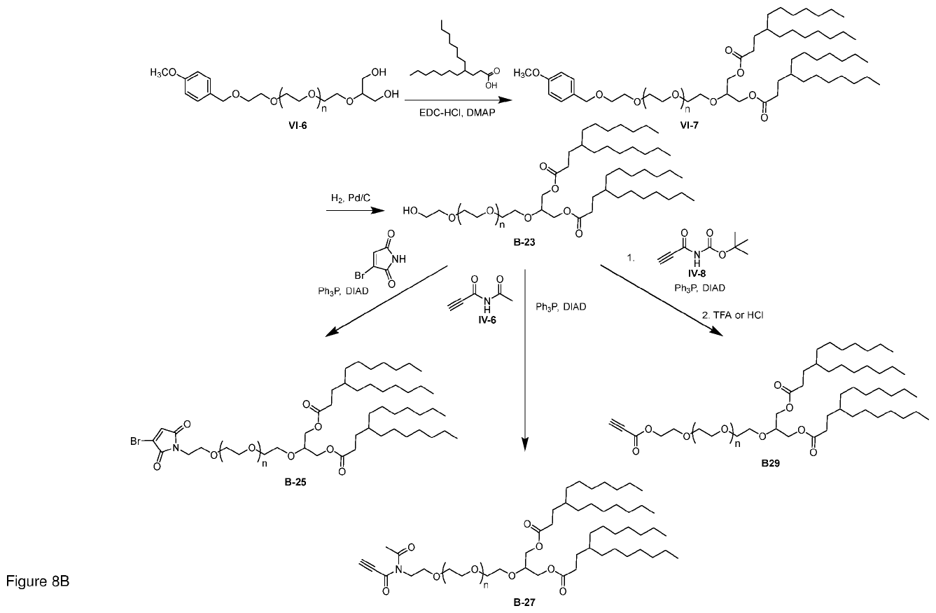

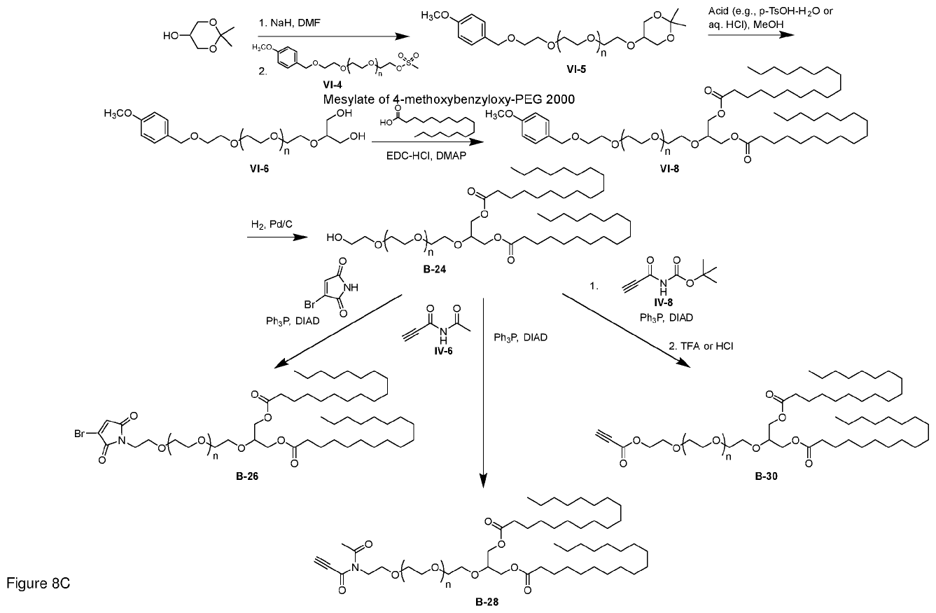

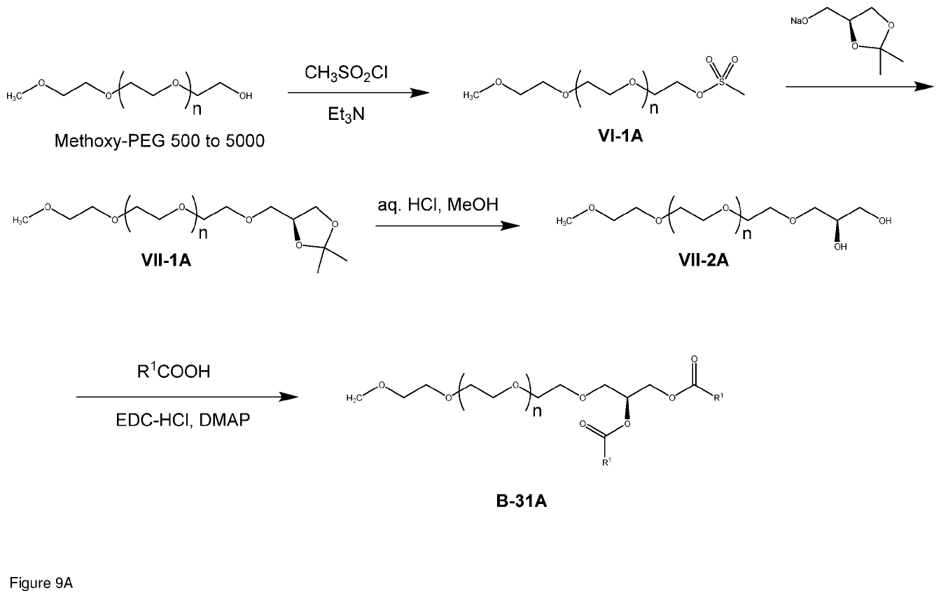

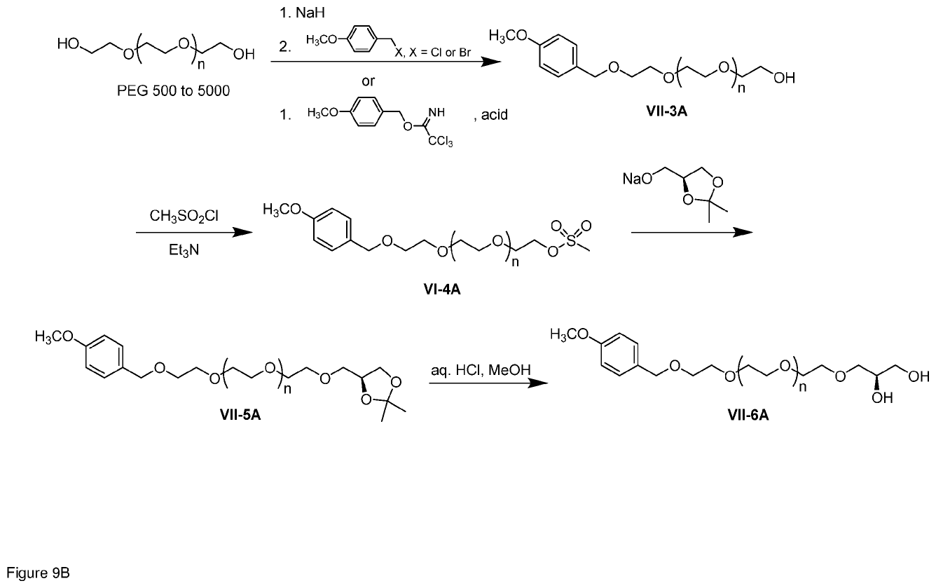

In some embodiments, the symmetrical di-ester PEG-lipid with a structure of formula PL-3 is functionalized with a maleimide, azide, alkyne, DBCO, bromo-maleimide, alkynylimide, or alkynylamide. In some embodiments, the PEG moiety is functionalized and the fatty acid esters are C₁₆-C₂₀ straight-chain alkyl fatty acids. In some embodiments having a C₁₄-C₂₀ symmetric branched-chain alkyl fatty acid ester, for example, the branched-chain alkyl fatty acid is C₁₄, C₁₅, C₁₆, C₁₇, C₁₈, C₁₉, or C₂₀. In some embodiments, the branch is at the 3rd, 4th, 5th, 6th, or 7th carbon position starting from the carbonyl of the fatty acid ester. In some embodiments, the symmetric di-ester PEG-lipid with a glycerol scaffold S3 or a structure of Formula PL-3 have a structure of Formula B-21 A (FIG. 7A), VI-7A (FIG. 8A), B-23A (FIG. 8A), B-25A (FIG. 8A), B-27A (FIG. 8A), B-29A (FIG. 8A), 18A (FIG. 12), 19A (FIG. 12), 20A (FIG. 12), 21A (FIG. 12), or 22A (FIG. 12). In some embodiments, the symmetric di-ester PEG-lipid with a glycerol scaffold S3 or a structure of Formula PL-3 is Compound VI-8 (FIG. 8C, Examples 10, 42 and 43), B-22 (FIG. 7B), B-24 (FIG. 8C, Example 26), 19 (Example 27), 20 (Example 28), 21 (Example 29), 22 (Example 30), B-26 (FIG. 8C), B-28 (FIG. 8C), or B-30 (FIG. 8C). In some embodiments, the symmetrical tri-ester PEG-lipid with a glycerol scaffold S3 or a structure of Formula PL-3 is Compound VI-7 (FIG. 8B, Examples 17, 42 and 43), B-21 (FIG. 7B), B-23 (FIG. 8B, Example 31), 24 (Example 32), 25 (Example 33), 26 (Example 34), 27 (Example 35), B-25 (FIG. 8B), B-27 (FIG. 8B), or B-29 (FIG. 8B).

The PEG-lipids commonly used in LNP can have a chiral glycerol scaffold and have straight-chain fatty acid ester tails or branched-chain fatty acid esters with the ester carbonyl in an a position to the branch point in the fatty acid ester tail and are connected to the PEG moiety by an ether linkage. In contrast to commercially available PEG-lipids, certain aspects of the instant disclosure provide an asymmetric glycerol-based PEG-lipid comprising 2 identical fatty acid esters that are C₁₄-C₂₀ symmetric branched-chain alkyl fatty acid esters. For example, the branched-chain alkyl fatty acid is C₁₄, C₁₅, C₁₆, C₁₇, C₁₈, C₁₉, or C₂₀. In some embodiments, the branch in the fatty acid ester tail is at the 3rd, 4th, 5th, 6th, or 7th carbon position starting from the carbonyl of the fatty acid ester, see for example, Formulas BFA-1, BFA-2, BFA-3, BFA-4, and BFA-5. In some embodiments, the carbonyls of the fatty acid esters are not positioned immediately adjacent to the branch point in the fatty acid ester tail (that is, the ester carbonyls are not in an a position relative to the branch point), for example they are in a β position relative to the branch point.

In some embodiments, asymmetric glycerol-based PEG-lipid has a structure of Formula PL-4

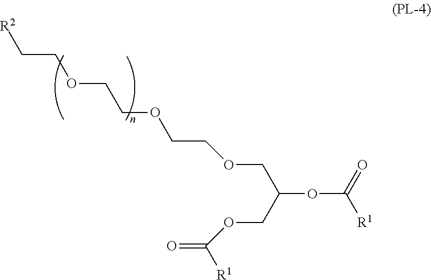

Wherein R¹, R² and n are defined the same as R¹, R² and n with respect to Formula PL-1 set forth supra.

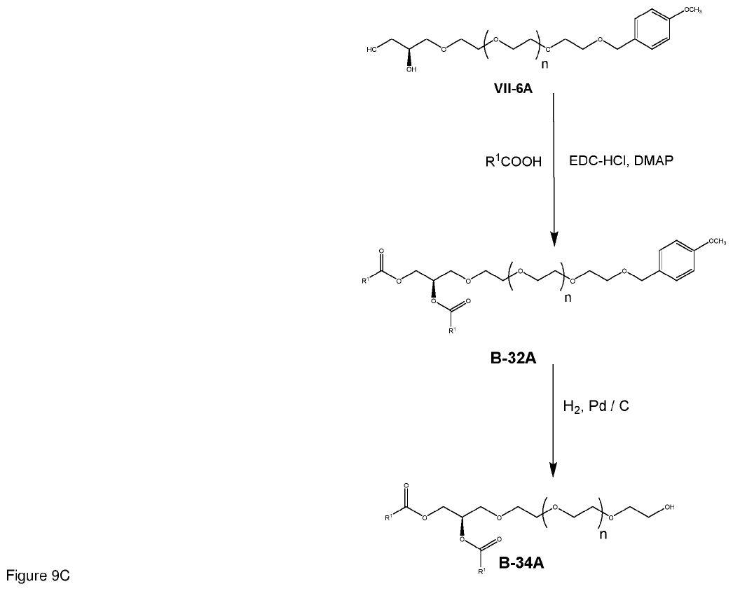

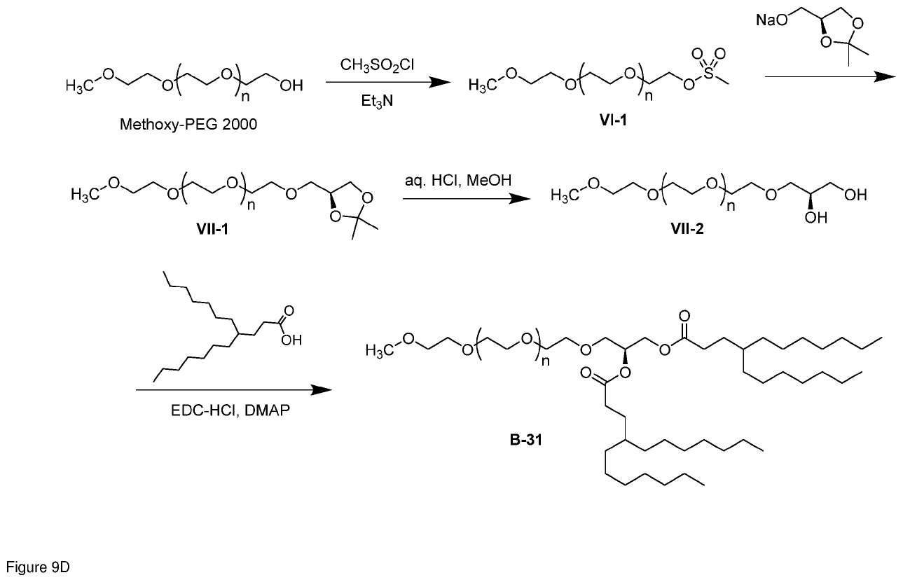

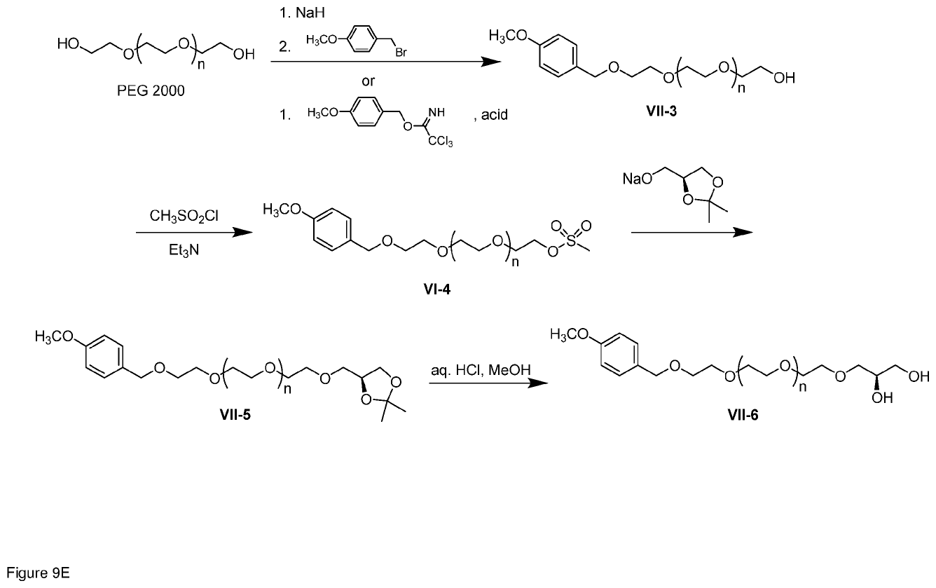

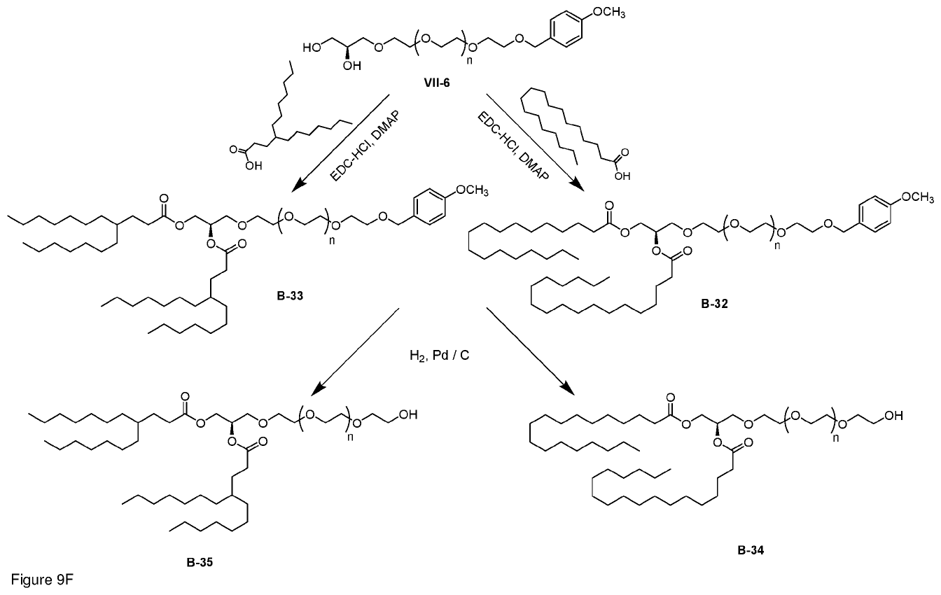

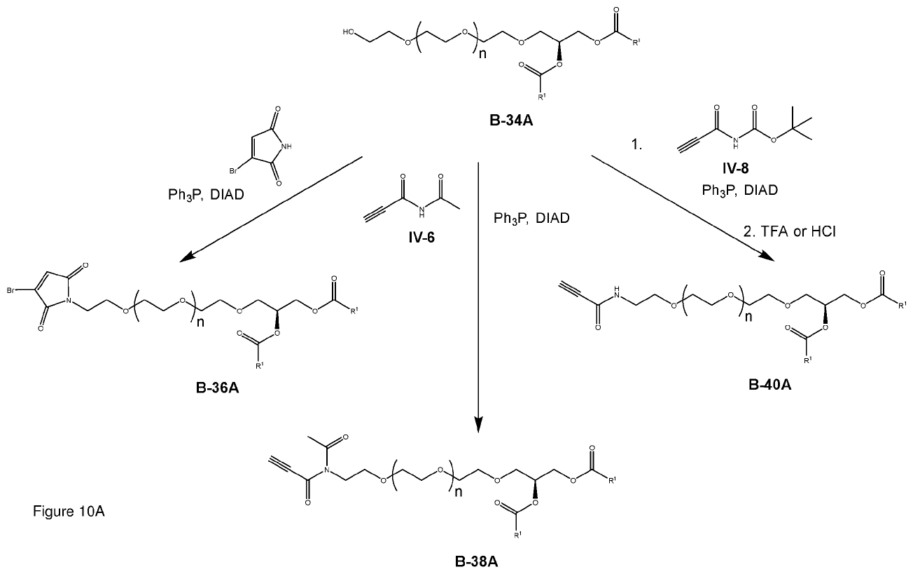

In some embodiments, the asymmetrical di-ester PEG-lipid with a structure of formula PL-4 is functionalized with a maleimide, azide, alkyne, DBCO, bromo-maleimide, alkynylimide, or alkynylamide. In some embodiments, the PEG moiety is functionalized and the fatty acid esters are C₁₄-C₂₀ or C₁₆-C₂₀ straight-chain alkyl fatty acids. In some embodiments having a branched-chain alkyl fatty acid ester, for example, the branched-chain alkyl fatty acid is C₁₄, C₁₅, C₁₆, C₁₇, C₁₈, C₁₉, or C₂₀. In some embodiments, the branch is at the 3rd, 4th, 5th, 6th, or 7th carbon position starting from the carbonyl of the fatty acid ester. In some embodiments, the asymmetrical di-ester PEG-lipids with a scaffold of formula S4 or a structure of Formula PL-4 have a structure of Formula B-31A (FIG. 9A), B-32A (FIG. 9C), B-34A (FIG. 9C), B-36A (FIG. 10A), B-38A (FIG. 10A), B-40A (FIG. 10A), 18A (FIG. 12), 19A (FIG. 12), 20A (FIG. 12), 21A (FIG. 12), or 22A (FIG. 12). In some embodiments, the asymmetrical di-ester PEG-lipid with a glycerol scaffold S4 or a structure of Formula PL-4 is Compounds B-31 (FIG. 9D), B-32 (FIG. 9F), B-33 (FIG. 9F), B-34 (FIG. 9F), B-35 (FIG. 9F), B-36 (FIG. 10B), B-37 (FIG. 10C), B-38 (FIG. 10B), B-39 (FIG. 10C), B-40 (FIG. 10B), or B-41 (FIG. 10C).

With respect to any aspect having a branched fatty acid chain, the fatty acid is a C₁₄ to C₂₀ fatty acid and has the structure of:

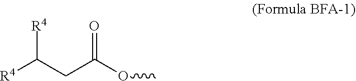

that is, the branch is at the 3rd carbon position starting from the carbonyl group, wherein R⁴ is C₆ to C₈ alkyl, or

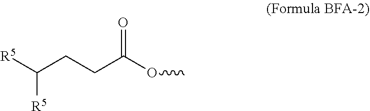

that is, the branch is at the 4th carbon position starting from the carbonyl group, wherein R⁵ is C₅ to C₈ alkyl, or

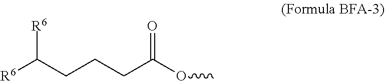

That is, the branch is at the 5th carbon position starting from the carbonyl group, wherein R⁶ is C₅ to C₇ alkyl, or

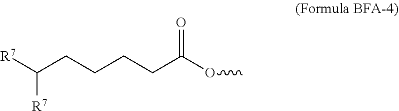

that is, the branch is at the 6th carbon position starting from the carbonyl group, wherein R⁷ is C₄ to C₇ alkyl, or

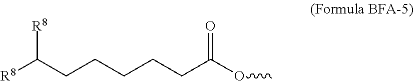

that is, the branch is at the 7th carbon position starting from the carbonyl group, wherein R⁸ is C₄ to C₆ alkyl, and [symbol] is the point of esterification to the scaffold. The partial structures presented herein generally show the oxygen atom of the ester in each of the partial structures to be joined. The combined structures should not be interpreted to indicate an O—O bond. Not having the branch immediately adjacent to the ester carbonyl reduces potential steric hinderance of the ester by one of the branches, facilitating biodegradability. In some embodiments, the C₁₄ to C₂₀ fatty acid is C₁₄, C₁₅, C₁₆, C₁₇, C₁₈, C₁₉, or C₂₀.

In some embodiments having a branched fatty acid chain, the fatty acid is a C₁₅ to C₁₉ fatty acid and has the structure of BFA-1. In some embodiments having a branched fatty acid chain, the fatty acid is a C₁₄ to C₂₀ fatty acid and has the structure of BFA-2. In some embodiments having a branched fatty acid chain, the fatty acid is a C₁₅ to C₁₉ fatty acid and has the structure of BFA-3. In some embodiments having a branched fatty acid chain, the fatty acid is a C₁₄ to C₂₀ fatty acid and has the structure of BFA-4. In some embodiments having a branched fatty acid chain, the fatty acid is a C₁₅ to C₁₉ fatty acid and has the structure of BFA-5. In further embodiments, the fatty acid size is any subset within the range of C₁₄ to C₂₀.

With respect to the above aspects, in some embodiments, the PEG moiety is PEG-500 to PEG-5000. In some instances, the PEG moiety is PEG-1000, PEG-2000, PEG-3000, PEG-4000, or PEG-5000. It is to be understood that polyethylene preparations of these sizes are polydisperse and that the nominal size indicates an approximate average molecular weight of the distribution. Taking the molecular weight of an individual repeating unit of (OCH₂CH₂)ₙ to be 44, a PEG molecule with n=11 would have a molecular weight of 502, n=22 would have a molecular weight of 986, with n=45 a molecular weight of 1998, and with n=113 a molecular weight of 4990. n=10 to 115 is used to represent PEG-lipids incorporating PEG moieties in the range of PEG-500 to PEG-5000 such as PEG-500, PEG-1000, PEG-1500, PEG-2000, PEG-2500, PEG-3000, PEG-3500, PEG-4000, PEG-4500, and PEG-5000, although some molecules from preparations at the average molecular weight boundaries will have n outside that range. For individual preparations n=11 is used to represent PEG-lipids incorporating PEG moieties from PEG-500, n=22 is used to represent PEG-lipids incorporating PEG moieties from PEG-1000, n=45 is used to represent PEG-lipids incorporating PEG moieties from PEG-2000 (thus, in the Examples PEG-2000 is represented by a structure with 45 units of OCH₂CH₂ but should not be interpreted as being any more exact than described here), n=67 is used to represent PEG-lipids incorporating PEG moieties from PEG-3000, n=90 is used to represent PEG-lipids incorporating PEG moieties from PEG-4000, n≈113 is used to represent PEG-lipids incorporating PEG moieties from PEG-5000. Some embodiments incorporate PEG moieties in a range bounded by any pair of the foregoing values of n or average molecular weight.

With respect to any of the PEG-lipids that have not been functionalized, in alternative embodiments, the PEG moiety of the PEG lipids may terminate with a methoxyl, a benzyloxyl, a 4-methoxybenzyloxyl, or a hydroxyl group (that is, an alcohol). The terminal hydroxyl facilitates functionalization. The methoxyl, benzyloxyl, and 4-methoxybenzyloxyl groups may be preferred for PEG-lipid that will be used as a component of the LNP without functionalization. However, all four of these alternatives are useful as the (non-functionalized) PEG-lipid component of LNPs. The 4-methoxybenzyloxyl group, often used as a protecting group during synthesis of the PEG-lipid, is readily removed to generate the hydroxyl group. Thus the 4-methoxybenzyloxyl group offers a convenient path to the alcohol when it is not synthesized directly. The alcohol is useful for being functionalized, prior to incorporation of the PEG-lipid into a LNP, so that a binding moiety can be conjugated to it as a targeting moiety for the LNP (making it a tLNP). As used herein, the terminus of the PEG moiety, and similar constructions, refers to the end of the PEG moiety that is not attached to the lipid.

With respect to the various PEG-lipid aspects, some embodiments specifically exclude one or more sizes or ranges of size of PEG, one or more PEG terminal groups, one or more functionalizations or conjugations, one or more sizes of fatty acid esters, straight-chain fatty acid esters, branched-chain fatty acid esters, one or more positions of branching for the branched-chain fatty acid esters, one or more positionings of the PEG moiety or fatty acid ester relative to a branch point (for example, not α, not β, or not γ), or any combination of such features. Other embodiments specifically include such features. For example, in some embodiments the fatty acid esters are straight-chain and/or not branched while in other embodiments they are branched and/or not straight-chain. Similarly, in some embodiments, the straight-chain fatty acid esters are not C₁₄.

The PEG-moiety provides a hydrophilic surface on the LNP, inhibiting aggregation or merging of LNP, thus contributing to their stability and reducing polydispersity. Accordingly, the newly disclosed PEG-lipid aspects constitute means for preventing aggregation. Some embodiments of means for preventing aggregation specifically exclude one or more of the above newly disclosed PEG-lipid aspects or embodiments. Some embodiments of means for preventing aggregation are specifically limited by one or another structural feature present in a subset of the above newly disclosed PEG-lipid aspects or embodiments.

Additionally, the PEG moiety impedes binding by the LNP to, for example, plasma proteins, including binding to apoE which is understood to mediate uptake of LNP by the liver, which can lead to an increase in the proportion of LNP reaching other tissues. The PEG-moiety can also be functionalized to serve as an attachment point for a targeting moiety. Conjugating a cell- or tissue-specific binding moiety to the PEG-moiety enables a tLNP to avoid the liver and bind to its target tissue or cell type, greatly increasing the proportion of LNP that reaches the targeted tissue or cell type. PEG-lipid can thus serve as means for inhibiting LNP binding to plasma proteins, functionalized PEG-lipid can serve as means for attaching or conjugating a binding moiety, and PEG-lipid conjugated to a binding moiety can serve as means for LNP-targeting. Some embodiments of means for preventing aggregation specifically exclude one or more of the above newly disclosed PEG-lipid aspects or embodiments. Some embodiments of means for inhibiting LNP binding or means for LNP-targeting are specifically limited by one or another structural feature present in a subset of the above newly disclosed PEG-lipid aspects or embodiments.

In certain aspects, the instant disclosure provides a method of synthesizing a symmetrical tri-ester PEG-lipid on a scaffold of formula S1. In certain embodiments, embodiments of the symmetrical tri-ester PEG-lipid having a scaffold of formula S1 has a structure of Formula B-1A and can be prepared according to the synthetic scheme of FIG. 1A. To synthesize the newly disclosed symmetrical tri-ester PEG-lipids, the ketone oxygen of dihydroxy acetone is converted to a blocked alkenyl ester (for example, with a t-butyl ester as in IV-1) and the double bond hydrogenated in the presence of Pd/C to provide IV-2 (see, for example, FIG. 1A). The hydroxyl groups of IV-2 are then reacted with a fatty acid (R¹—COOH), for example, in the presence of EDC-HCl and DMAP in triethylamine. The blocking protection group of carboxylic acid in IV-1 is then removed, for example, with TFA, generating an acid which is then coupled with a PEG moiety of 500-5000 MW (FIG. 1A), for example PEG-2000 (FIG. 1B), and modified PEG such as methoxy-PEG, benzyloxy-PEG, or 4-methoxybenzyloxy-PEG-500 to 5,000 (VII-3A) or PEG-2000 (VII-3) to generate analogues in which the PEG moiety terminates with a methoxyl, benzyloxyl, or 4-methoxybenzyloxyl group, respectively (FIG. 11A). FIGS. 1B and 11B depict syntheses of embodiments of Formulas B-1A, B-42A, B-44A, and B-46A, wherein R₁COOH is a C₁₈ fatty acids, straight-chain (stearic acid for Compounds B-2, B-42, B-44, and B-46) or branched (4-heptylundecanoic acid for Compounds B-1, B-43, B-45, and B-47). 4-Methoxybenzyl group is readily removed to generate the corresponding alcohol of Formula B-1A, e.g., Compounds B-1 and B-2, which can then be functionalized, (e.g., FIGS. 3A-C, and 4A-C).

For straight-chain fatty acids, in some embodiments, myristic (C₁₄), pentadecanoic (C₁₅), palmitic (C₁₆), heptadecanoic (C₁₇), nonadecanoic (C₁₉), or eicosanoic (C₂₀) acids can be substituted for the stearic acid. For symmetric branched-chain fatty acids (that is fatty acids with equal numbers of carbons emanating from each side of the branch point), in some embodiments the fatty acid 4-pentylnonanoic acid (C₁₄), 4-hexyldecanoic acid (C₁₆), or 4-octyldodecanoic acid (C₂₀) can be substituted for 4-heptylundecanoic acid, that is, other branched fatty acids with a structure of BFA-2 (C₁₄, C₁₆, C₁₈, or C₂₀). In further embodiments, C₁₄ to C₂₀ symmetric branched fatty acids that branch at the 3rd, 5th, 6th, or 7th carbon position starting from the carbonyl group, as described above, can be substituted for the 4-heptylundecanoic acid, generating symmetric branched fatty acid tails. In some embodiments, the symmetric branched fatty acid tails are generated by using a fatty acid with a structure of Formula BFA-1 (C₁₅, C₁₇, or C₁₉), BFA-3 (C₁₅, C₁₇, or C₁₉), BFA-4 (C₁₄, C₁₆, C₁₈, or C₂₀), or BFA-5 (C₁₅, C₁₇, or C₁₉), again, generating symmetric branched fatty acid tails.

In certain aspects, the instant disclosure provides a method of synthesizing a symmetrical di-ester PEG-lipid having a scaffold S2 from 2-(hydroxymethyl)butane-1,4-diol according to the synthetic scheme of FIGS. 5A-D. To synthesize the newly disclosed symmetric di-ester PEG-lipids on a scaffold derivable from 2-(hydroxymethyl)butane-1,4-diol the diol undergoes an acetal exchange with 4-methoxybenzaldehyde dimethyl acetal, for example, in the presence of p-TsOH in THF to provide V-1 (FIG. 5A). Bromination with carbon tetrabromide and triphenyl phosphine in DMF affords bromide V-2 which is coupled with methoxy-PEG (e.g., methoxy PEG 500-5000, FIG. 5A; methoxy-PEG-2000, FIG. 5C) in the presence of NaH in DMF or KOtBu in THF to yield V-3A (FIG. 5A) or V-3 (FIG. 5C). Bis-esterification with an appropriate fatty acid, as described herein, in the presence of EDC-HCl and DMAP in CH₂Cl₂ affords PEG-lipids in which the PEG moiety terminates with a methoxyl group. Examples include PEG-lipids of Formula B-9A (FIG. 5A), and embodiments of Formula B-9A: Compounds B-9, with branched chain fatty acid esters, and B-10, with straight chain fatty acid esters (FIG. 5C).

Alternatively, synthesis of these symmetric di-ester PEG-lipids proceeds by coupling of V-2 with 4-methoxybenzyloxy-PEG (e.g., 4-methoxybenzyloxy-PEG 500 to 5000, FIG. 5B; 4-methoxybenzyloxy-PEG-2000, FIG. 5D) to yield V-5A (FIG. 5B) or V-5 (FIG. 5D). Analogous bis-esterification results in PEG-lipids in which the PEG moiety terminates in a 4-methoxybenzyloxyl group. Examples include PEG-lipids of Formula B-11 A (FIG. 5B), and embodiments of Formula B-11 A: Compounds B-11 and B-12. 4-Methoxybenzyl group is readily removed to generate the corresponding alcohol, which can then be functionalized, by, e.g. reaction with hydrogen in the presence of Pd/C in ethyl acetate (FIGS. 6A-C).

In certain aspects, the present disclosure provides a method of synthesizing a symmetrical di-ester PEG-lipid on a symmetric glycerol-derived scaffold S3 in which an esterified PEG moiety is attached to a central position and two identical fatty acids are esterified to two end positions of the scaffold according to the synthetic scheme of FIGS. 7A-B. To synthesize the newly disclosed symmetric glycerol-based lipids, the sodium salt of 2,2-dimethyl-1,3-dioxan-5-ol is reacted with a mesylate of methoxy-PEG (for example, mesylate of methoxy-PEG 500 to 5000, Formula VI-1A, FIG. 7A; and mesylate of methoxy-PEG 2000, VI-1; FIG. 7B) in the presence of sodium hydride in DMF to provide the acetonide. Acetonide hydrolysis (aq. HCl, MeOH) then affords a diol which is bis-esterified with an appropriate fatty acid, as described herein, in the presence of EDC-HCl and DMAP in CH₂Cl₂ to afford PEG-lipids in which the PEG moiety terminates with a methoxyl group, Formula B-21A (FIG. 7A). Examples include PEG-lipids of Formula B-21A (FIG. 7A), and embodiments of Formula B-21A: Compounds B-21, with branched chain fatty acid esters, and B-22, with straight chain fatty acid esters (FIG. 7B).

To synthesize the 4-methoxybenzyl ether and alcohol analogs of these symmetric glycerol-based lipids the mesylate of 4-methoxybenzyloxy-PEG is substituted for the mesylate of methoxy-PEG of the above reaction (FIGS. 8A-C). Examples of the 4-methoxybenzyl ethers include PEG-lipids of Formula VI-7A (FIG. 8A), and embodiments of Formula VI-7A: VI-7, with branched-chain fatty acid esters, and VI-8, with straight-chain fatty acid esters. To generate the alcohols, these ethers are reacted with hydrogen in the presence of Pd/C in ethyl acetate (FIGS. 8A-C). Examples of the alcohols include PEG-lipids of Formula B-23A (FIG. 5A), and embodiments of Formula B-23A: Compounds B-23 (FIG. 5B), with branched-chain fatty acid esters, and B-24 (FIG. 5C), with straight-chain fatty acid esters. PEG-lipids of Formula B-23A and Compounds B-23 and Compounds B-24 may further be functionalized as shown in FIGS. 8A-C.

In certain aspects, the instant disclosure provides a method of synthesizing an asymmetric glycerol-based PEG-lipid with scaffold S4 with 2 fatty acids that are C₁₄-C₂₀ symmetric branched-chain alkyl fatty acids according to the synthetic scheme of FIGS. 9A&D. To synthesize the newly disclosed asymmetric glycerol-based lipids in which the PEG moiety terminates with a methoxyl group, methoxy-PEG is reacted with methanesulfonyl chloride (Et3N, THF) to yield mesylate, e.g., mesylates of Formula VI-1A (PEG-500 to 5000, FIG. 9A) or its analogues based on different sized PEG (e.g., Compound VI-1, mesylate of methoxy-PEG-2000, FIG. 9D). The mesylate is then reacted with the sodium salt of (S)-2,2,-dimethyl-dioxolane-4-methanol, produced by reaction with sodium hydride in DMF, to give the acetonide (for example, acetonides of Formula VII-1A in FIG. 9A, Compound VII-1 in FIG. 9D). Acetonide hydrolysis with aq. HCl in methanol provides the diol (such as diols of Formula VII-2A in FIG. 9A; and Compound VII-2 in FIG. 9D). Bis-esterification of the diol with an appropriate fatty acid, as described herein, in the presence of EDC-HCl and DMAP in CH₂Cl₂ affords PEG-lipids in which the PEG moiety terminates with a methoxyl group (e.g., PEG-lipids of Formula B-31 A in FIG. 9A; and Compound B-31 in FIG. 9D). Unlike the symmetric lipids with a glycerol scaffold above, this synthesis provides a PEG moiety that is attached at the position of one of glycerol's primary hydroxyls.

To synthesize the 4-methoxybenzyl ether and alcohol analogs of these asymmetric glycerol-based lipids, a PEG sodium salt is reacted with 4-methoxybenzyl bromide and sodium hydride in DMF (FIG. 9B) to give Formula VII-3A (FIG. 9B) or Compound VII-3 (FIG. 9E) or an analogue based on different sized PEG. Alternatively, the same molecules can also be formed by reacting PEG with 4-methoxybenzyl trichloroacetimidate in the presence of CF₃SO₃H in THF. The compound can then be converted to the mesylate by treatment with methanesulfonyl chloride and triethylamine in THF. The mesylate is then reacted with the sodium salt of (S)-2,2-dimethyl-dioxolane-4-methanol, formed by reaction with NaH in DMF, to give an acetonide (such as, acetonides of Formula VII-5A in FIG. 98; Compound VII-5 in FIG. 9E). Acetonide hydrolysis with aq. HCl in methanol provides a diol (such as, diols of Formula VII-6A in in FIG. 9B; Compound VII-6 in FIG. 9E). Bis-esterification of the diol with an appropriate fatty acid, as described herein, in the presence of EDC-HCl and DMAP in CH₂Cl₂ affords PEG-lipids in which the PEG moiety terminates with a 4-methoxybenzyloxyl group. Examples include PEG-lipids of Formula B-32A (FIG. 9C), and embodiments of Formula B-32A: compounds B-32, with straight-chain fatty acid esters and B-33, with branched-chain fatty acid esters (FIG. 9F). To generate the alcohols, these ethers are reacted with hydrogen in the presence of Pd/C in ethyl acetate (FIG. 9C). Examples of the alcohols include PEG-lipids of Formula B-34A (FIG. 9C), and embodiments of Formula B-34A: Compounds B-35, with branched-chain fatty acid esters, and B-34, with straight-chain fatty acid esters (FIG. 9F).

Functionalized PEG-lipids are obtained from the alcohol form (of the PEG moiety). Several possible functionalization reactions are well-known in the art.

One of the most widely used reactions for conjugating antibodies and other binding moieties to other molecules is the maleimide reaction (see Parhiz et al., Journal of Controlled Release 291:106-115, 2018). Also popular is so-called click chemistry (see Kolb et al., Angewandte Chemie International Edition 40(11):2004-2021, 2001; and Evans, Australian Journal of Chemistry 60(6):384-395, 2007). In some embodiments, conjugation of the binding moiety to the PEG is accomplished by maleimide SATA chemistry. The SATA introduction is made to the antibody (on any solvent accessible lysine), then the thioacetyl group on SATA is deprotected (for example, with hydroxylamine) and the resulting thiol (SH) is conjugated to maleimide group carried on the PEG-lipid in the LNP. In other embodiments, conjugation of the binding moiety to the PEG is accomplished through cysteine (Cys) by site directed conjugation. Reagents for such reactions include Lipid-PEG-maleimide, lipid-peg-cysteine, lipid-PEG-alkyne, and lipid-PEG-azide. In the context of targeted lipid nanoparticles (tLNP) comprising nucleic acid to be delivered into cells, the maleimide reaction has been used to form a covalent bond between a thiol in the antibody (or other binding moiety) and a functionalized polyethylene glycol (PEG) unit in a PEG-lipid. On the binding moiety side of the reaction one can use an existing cysteine sulfhydryl, or derivatize the protein by adding a sulfur containing carboxylic acid, for example, to the epsilon amino of a lysine to react with a maleimide, bromomaleimide or bromomaleimide amide, alkynoic amide, or alkynoic imide. Similarly, a binding moiety polypeptide can be engineered to contain an N- or C-terminal extension comprising an accessible thiol group. Alternatively, one can add an alkyne or an azide to a sulfhydryl or an epsilon amino of a lysine to participate in a click chemistry reaction. Then the PEG-lipid would carry with it either the azide or the alkyne necessary to participate in a click reaction.

There are also several approaches to site-specific conjugation. Particularly but not exclusively suitable for truncated forms of antibody, C-terminal extensions of native or artificial sequences containing a particularly accessible cysteine residue are commonly used. Partial reduction of cystine bonds in the antibody can also generate thiol groups for site-specific conjugation. Alternatively, the C-terminal extension can contain a sortase A substrate sequence, LPXTG (SEQ ID NO: 1) which can then be functionalized in a reaction catalyzed by sortase A and conjugated to the PEG-lipid, including through click chemistry reactions (see, for example, Moliner-Morro et al., Biomolecules 10(12):1661, 2020 which is incorporated by reference herein for all that it teaches about antibody conjugations mediated by the sortase A reaction and/or click chemistry). For whole antibody and other forms comprising an Fc region, site-specific conjugation to either (or both) of two specific lysine residues (Lys248 and Lys288) can be accomplished without any change to or extension of the native antibody sequence by use of one of the AJICAP® reagents (see, for example, Matsuda et al., Molecular Pharmaceutics 18:4058-4066, 2021 and Fujii et al., Bioconjugate Chemistry https://doi.org/10.1021/acs.bioconjchem.3c00040, 2023, which are incorporated by reference herein for all that they teach about conjugation of antibodies with AJICAP reagents). The AJICAP reagents are modified affinity peptides that bind to specific loci on the Fc and react with an adjacent lysine residue. The peptide is then cleaved with base to leave behind a thiol-functionalized lysine residue which can then undergo conjugation through maleimide or haloamide reactions, for example). Functionalization with azide or dibenzocyclooctyne (DBCO) for conjugation by click chemistry is also possible.

Accordingly, in some embodiments the binding moiety is conjugated to the PEG moiety of the PEG-lipid through a thiol modified lysine residue. In some embodiments the conjugation is through a cysteine residue in a native or added antibody sequence. In other embodiments, the conjugation is through a sortase A substrate sequence. In still other embodiments, the conjugation is through a specific lysine residue (Lys248 or Lys288) in the Fc region.

The binding moiety of the tLNP directs the tLNP to the cell or tissue intended to receive the nucleic acid component. However, this conjugation is subject to a reverse Michael reaction releasing the binding moiety, potentially causing loss of targeting and increased delivery of the nucleic acid to non-target tissue, especially the liver. Additionally, PEG-lipids with shorter fatty acid esters, for example, straight-chain fatty acid esters of C₁₄ or less, are subject to shedding of the PEG-lipid from the LNP at rates that could also impair targeting by the conjugated binding moiety.

The structure of a standard PEG-DMG lipid is shown below as E. The core of this lipid is a glycerol moiety which is brought into the synthesis as a racemic fragment, thus the lipid is a mixture of optical antipodes. Additionally, the connectivities of the fragments are a mixture of an ether bond to the PEG and ester bonds to the 14-carbon myristic acid, requiring alternate chemistry and endowing the lipid with differential reactivity and stability issues during synthesis. Ensuring that the PEG is connected to a primary hydroxy of the glycerol and the myristic acids are connected to the remaining primary OH as well as the secondary-OH of glycerol are issues to be confronted and dealt with when employing a non-symmetrical triol, such as glycerol, in the synthesis of E.

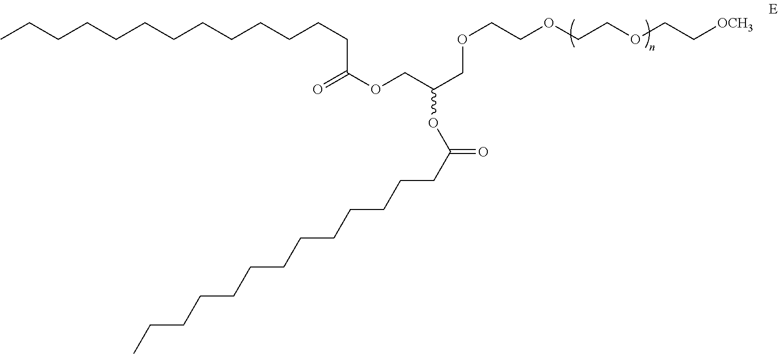

A symmetric tri-ester PEG-lipid described herein, such as PEG-lipids of Formula B-1A (FIG. 1A) and Compounds B-1 and B-2 (see FIG. 1B), employs a symmetrical central linking fragment, which is not chiral and facilitates preparation of pure products as there will be no differential reactivity at the two hydroxyls where the fatty acids will be esterified. The ester linkage of the PEG moiety may also contribute to the biodegradability of the compound. The advantage of symmetry is also obtained with the symmetrical diesters such as PEG-lipids of Formula B-13A (FIG. 6A) and Compounds B-13, B-15, B-17, and B19 (FIG. 6B), as well as Formula B-23A (FIG. 8A) and Compounds B-23, B-25, B-27, and B-29 (FIG. 8B). The methods of synthesis also provide the opportunity to carry a variety of acids into the coupling reaction.

In the typical maleimide reaction a maleimide moiety on a functionalized PEG-lipid captures a sulfhydryl moiety resident on the targeting entity (often an antibody or containing the antigen binding domain thereof). The addition to a maleimide, in a Michael reaction, affords a 3-RS-succinimide such as A (below), which is reactive and susceptible to a retro-Michael process, in a reverse reaction, to regenerate the maleimide and decouple the targeting moiety from the LNP.

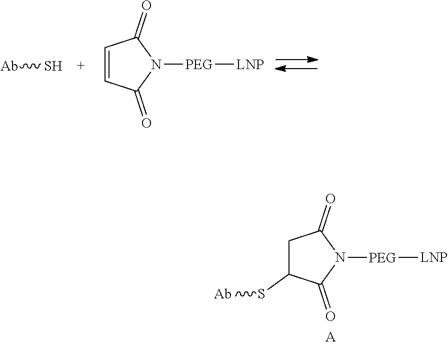

The bromomaleimide or bromomaleimide amide, a alkynoic amide, and alkynoic imide linking entities described herein are incapable of undergoing the retro-Michael reaction, giving B (a thiomaleimide), C (a thioacrylamide), and D (a thio-N-acetyl acrylamide), respectively. Therefore, they will carry the targeting moiety with much less loss.

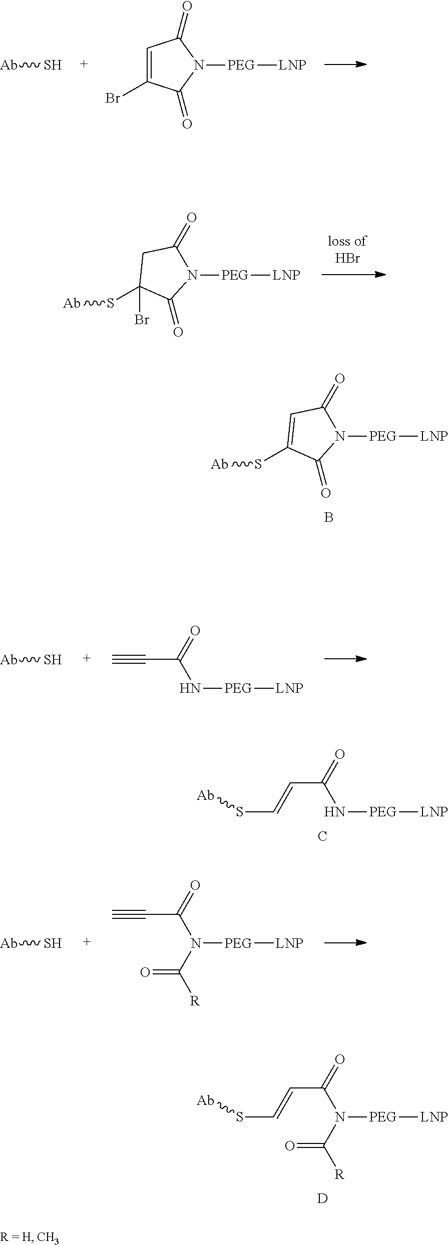

Accordingly, in certain aspects, the instant disclosure provides a functionalized PEG-lipid comprising a bromomaleimide or bromomaleimide amide moiety, an alkynylamide moiety, or an alkynylimide moiety at the terminal hydroxyl end of the PEG moiety.

Some embodiments provide a functionalized PEG-lipid comprising a bromomaleimide moiety at the terminal hydroxyl end of the PEG moiety. Instances of this embodiment include functionalized PEG-lipids of Formula B-3A, Formula B-15A, Formula B-25A, Formula B-36A, Formula 22A, Compounds B-3, B-4, B-15, B-16, B-25, B-26, B-36, B-37, 22, and 27. Other embodiments provide a functionalized PEG-lipid comprising a bromomaleimide amide moiety at the terminal end of the PEG moiety in which the hydroxyl had been converted to an amine. Instances of this embodiment include 22, 27, 31, and 34.

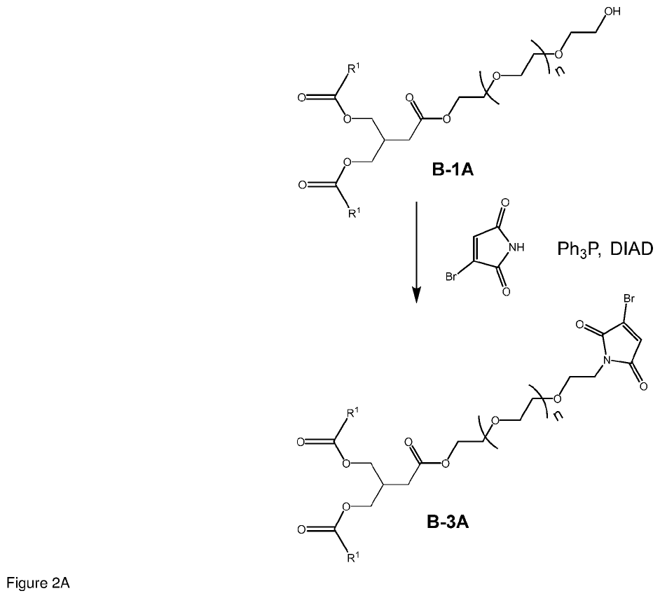

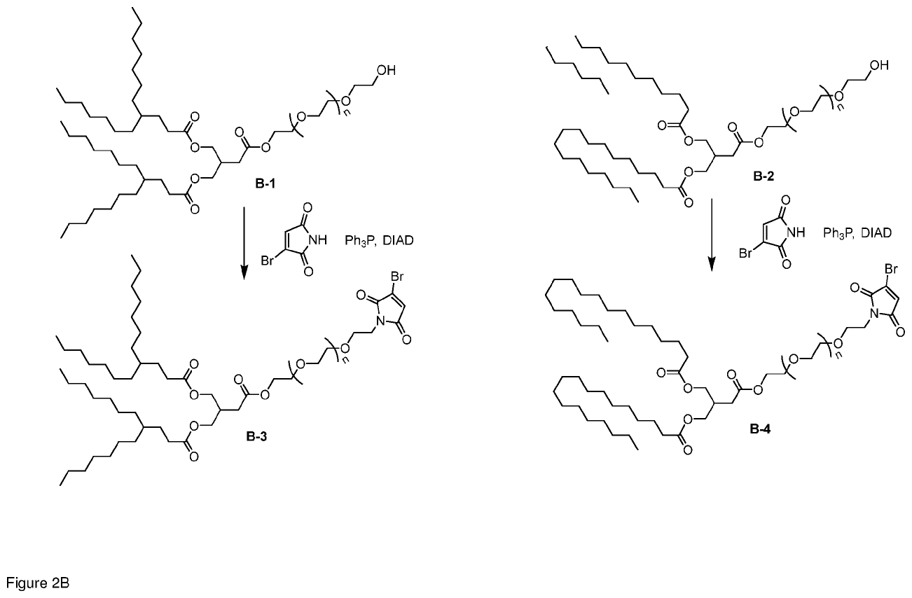

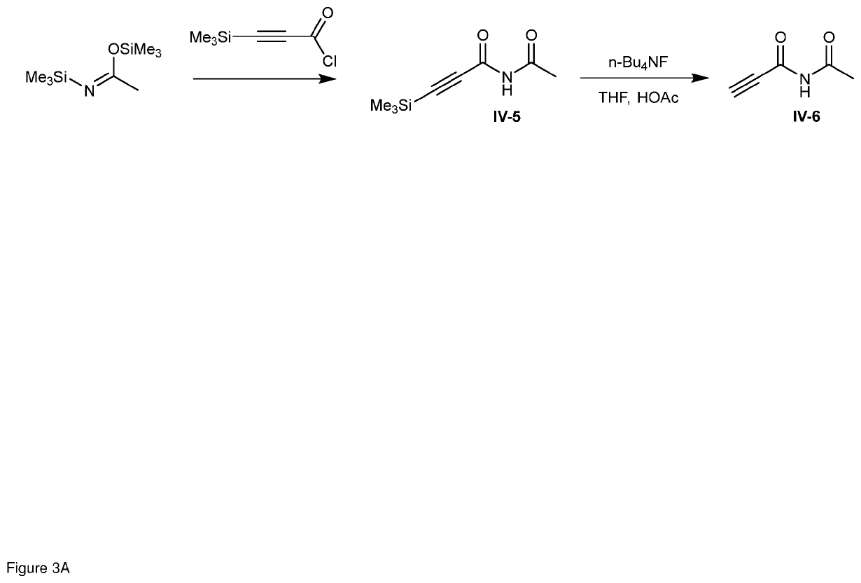

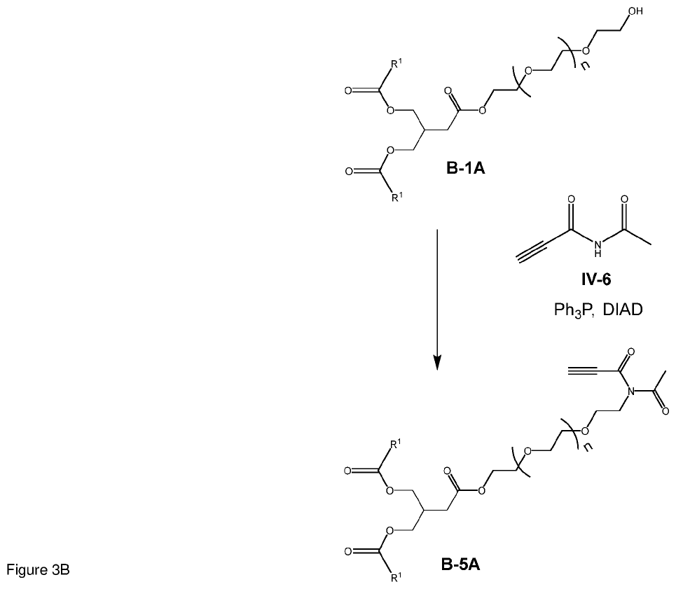

In certain aspects, the instant disclosure provides a method of functionalizing a PEG-lipid so that the PEG-lipid has a bromomaleimide moiety appended to the terminal hydroxyl end of the PEG moiety. To functionalize the PEG-lipid for eventual conjugation with a binding moiety, the terminal hydroxyl end of the PEG moiety is reacted with bromomaleimide under Mitsunobu conditions according to the synthetic scheme of FIGS. 2A-B to produce the functionalized (bromomaleimide) species. The reaction with bromomaleimide is also shown with alternative PEG-lipids in FIGS. 6A-C, 8A-C, 10A-C, and 12.

One embodiment is a functionalized PEG-lipid comprising an alkynylimide moiety at the terminal hydroxyl end of the PEG moiety. Instances of this embodiment include functionalized PEG-lipids of Formula B-5A, Formula B-17A, Formula B-27A, Formula B-38A, Compounds B-5, B-6, B-17, B-18, B-27, B-28, B-38, and B-39.

In certain aspects, the instant disclosure provides a method of functionalizing a PEG-lipid so that the PEG-lipid has an alkynylimide moiety appended to the terminal hydroxyl end of the PEG moiety. To make PEG-lipids with this functionalization the terminal hydroxyl end of the PEG moiety is reacted with N-acetylpropiolamide (IV-6) under Mitsunobu conditions according to the synthetic scheme of FIGS. 3B&C. to produce the alkynoic imide functionalized species. The reaction with IV-6 is also shown with alternative PEG-lipids in FIGS. 6A-C, 8A-C, and 10A-C.

One embodiment is a functionalized PEG-lipid comprising an alkynylamide moiety appended to the terminal hydroxyl end of the PEG moiety. Instances of this embodiment include functionalized PEG-lipids of Formula B-7A, Formula B-19A, Formula B-29A, Formula B-40A, Compounds B-7, B-8, B-19, B-20, B-29, B-30, B-40, and B-41.

In certain aspects, the present disclosure provides a method of functionalizing a PEG-lipid so that the PEG-lipid has an alkynylamide moiety at the terminal hydroxyl end of the PEG moiety. To make PEG-lipids with this functionalization the terminal hydroxyl end of the PEG moiety is reacted with tert-butyl propioloylcarbamate (IV-8) under Mitsunobu conditions according to the synthetic scheme of FIGS. 4B&C to produce the alkynoic amide functionalized species after removal of the protecting group. The reaction with IV-8 is also shown with alternative PEG-lipids in FIGS. 6A-C, 8A-C, and 10A-C.

As used herein, lipid nanoparticle (LNP) means a solid particle, as distinct from a liposome having an aqueous lumen. The core of a LNP, like the lumen of a liposome, is surrounded by a layer of lipid that may be, but is not necessarily, a continuous lipid bilayer as found in a liposome.

In certain aspects, the instant disclosure provides a LNP comprising a symmetrical PEG-lipid in which a PEG-moiety is attached to a central position on a scaffold and two identical fatty acids are esterified to two end positions on the scaffold. In some embodiments, the PEG-lipid is functionalized. In some instances, the functionalization is a maleimide or a triazole formed from a click chemistry reaction. In other instances, the functionalization is a bromomaleimide or bromomaleimide amide, an alkynoic amide, or an alkynoic imide. In some embodiments, the functionalized PEG-lipid has been conjugated to a binding moiety so that the LNP is a tLNP.

In some embodiments, the LNP or tLNP comprises a symmetrical PEG-lipid that is a tri-ester PEG-lipid in which an esterified PEG moiety is attached to a central position on a scaffold and two identical fatty acids are esterified to two end positions on the scaffold. In some embodiments, the fatty acid esters are not positioned immediately adjacent to the branch point, for example they are in a β position relative to the branch point. In some embodiments, the esterified PEG moiety is not positioned immediately adjacent to the branch point, for example it is in a β or γ position relative to the branch point. In some embodiments, the scaffold has the structure S1

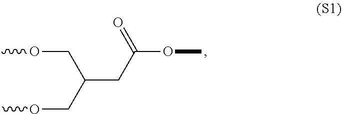

where [symbol] represents the points of esterification with fatty acid and [symbol] represents esterification with the PEG moiety. In some embodiments, the fatty acid esters are C₁₄-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the PEG moiety is functionalized and the fatty acid esters are C₁₆-C₂₀ straight-chain alkyl fatty acids, e.g., C₁₆, C₁₇, C₁₈, C₁₉, or C₂₀ straight-chain alkyl fatty acids. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. In some embodiments, the branch is at the 3, 4, 5, 6, or 7 position of the fatty acid ester. In some instances, the tri-ester PEG-lipid has a structure of Formula B-1A, B-42A, B-44A, or B-46A, In some instances, the tri-ester PEG-lipid is Compound B-1, B-43, B-45, B-47, B-2, B-42, B-44, or B-46. In some instances, the tri-ester PEG-lipid is functionalized, for example, PEG-lipids of Formula B-3A and Compounds B-3 or B-4 (bromomaleimide), PEG-lipids of Formula B-5A and Compounds B-5 or B-6 (alkynoic imide), or PEG-lipids of Formula B-7A and Compounds B-7 or B-8 (alkynoic amide). In some embodiments, the LNP further comprises a symmetrical di-ester PEG-lipid newly disclosed herein. In some embodiments, the LNP further comprises an asymmetric PEG-lipid newly disclosed herein. In some embodiments, the functionalized PEG-lipid has been conjugated to a binding moiety so that the LNP is a tLNP.

In some embodiments, the LNP or tLNP comprises a symmetrical PEG-lipid that is a di-ester PEG-lipid in which a PEG-moiety is attached to a central position on a scaffold by an ether linkage and two identical fatty acids are esterified to two end positions on the scaffold. In some embodiments, the fatty acid esters are not positioned immediately adjacent to the branch point, for example they are in a β position relative to the branch point. In some embodiments, the PEG moiety is not positioned immediately adjacent to the branch point, for example it is in a β or γ position relative to the branch point. In some embodiments, the scaffold has the structure S2

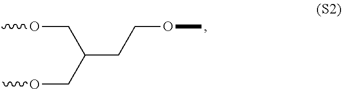

Where [symbol] represents the points of esterification of the fatty acid and [symbol] represents the ether linkage connection to the PEG moiety. In some embodiments, the fatty acid esters are C₁₆-C₂₀ straight-chain alkyl fatty acids. In some embodiments, the fatty acid esters are C₁₄-C₂₀ symmetric branched-chain alkyl fatty acids. In some embodiments, the branch is at the 3, 4, 5, 6, or 7 position of the fatty acid ester. In some instances, the di-ester PEG-lipid has a structure of Formula B-9A, B-11 A, or B-13A. In some instances, the di-ester PEG-lipid is Compound B-9, B-10, B-11, B-12, B-13, or B-14. In some instances, the tri-ester PEG-lipid is functionalized, for example, PEG-lipids of Formula B-15A and Compounds B-15 or B-16 (bromomaleimide), PEG-lipids of Formula B-17A and Compounds B-17 or B-18 (alkynoic imide), or PEG-lipids of Formula B-19A and Compounds B-19 or B-20 (alkynoic amide). In some embodiments, the LNP further comprises a symmetrical tri-ester PEG-lipid, or a symmetrical di-ester PEG-lipid with a glycerol scaffold, newly disclosed herein. In some embodiments, the LNP further comprises an asymmetric PEG-lipid newly disclosed herein. In some embodiments, the functionalized PEG-lipid has been conjugated to a binding moiety so that the LNP is a tLNP.

In some embodiments of the LNP or tLNP, the symmetrical PEG-lipid is a symmetrical di-ester PEG-lipid, in which a PEG-moiety is attached to a central position on a glycerol scaffold by an ether linkage and two identical fatty acids are esterified to two end positions on the glycerol scaffold. In some embodiments, the fatty acid esters are not positioned immediately adjacent to the branch point, for example they are in a β position relative to the branch point. In some embodiments, the PEG moiety is not positioned immediately adjacent to the branch point, for example it is in a β or γ position relative to the branch point. In some embodiments, the scaffold is a glycerol scaffold having the structure S3

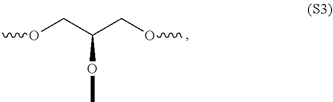

where [symbol] represents the points of esterification of the fatty acids and [symbol] represents the ether linkage connection to the PEG moiety. In some embodiments, the fatty acid esters are C₁₆-C₂₀ straight-chain alkyl, e.g., C₁₆, C₁₇, C₁₈, C₁₉, or C₂₀ straight-chain alkyl fatty acids. In some embodiments, the fatty acid esters are C₁₄-C₂₀ symmetric branched-chain alkyl. For example, the branched-chain alkyl fatty acid is C₁₄, C₁₅, C₁₆, C₁₇, C₁₈, C₁₉, or C₂₀. In some embodiments, the branch is at the 3, 4, 5, 6, or 7 position of the fatty acid ester. In some instances, the di-ester PEG-lipid has a structure of Formula B-21A, VI-7A, or B-23A. In some instances, the di-ester PEG-lipid is Compound B-21, B-22, VI-7, B-23, VI-8, or B-24. In some instances, the symmetrical di-ester PEG-lipid with an S3 scaffold is functionalized, for example, PEG-lipids of Formula B-25A and Compounds B-25 or B-26 (bromo-maleimide), PEG-lipids of Formula B-27A and Compounds B-27 or B-28 (alkynoic imide), or PEG-lipids of Formula B-29A and Compounds B-29 or B-30 (alkynoic amide). In some embodiments, the LNP further comprises a symmetrical tri-ester PEG-lipid, or a symmetrical di-ester PEG-lipid with a S2 scaffold, newly disclosed herein. In some embodiments, the LNP further comprises an asymmetric PEG-lipid newly disclosed herein. In some embodiments, the functionalized PEG-lipid has been conjugated to a binding moiety so that the LNP is a tLNP.

In certain aspects, the instant disclosure provides a LNP comprising an asymmetric glycerol-based PEG-lipid comprising 2 fatty acid esters that are C₁₄-C₂₀ symmetric branched-chain alkyl fatty acids in which the PEG moiety is attached at the position of one of glycerol's primary hydroxyls groups by an ether linkage. For example, the branched-chain alkyl fatty acid is C₁₄, C₁₅, C₁₆, C₁₇, C₁₈, C₁₉, or C₂₀. In some embodiments, the fatty acid esters are not positioned immediately adjacent to the branch point (are not in an a position relative to the branch point), for example they are in a β position relative to the branch point. In some embodiments, the PEG moiety is not positioned immediately adjacent to the branch point, for example it is in a β position relative to the branch point. In some embodiments, the branch in the fatty acid ester tail is at the 3, 4, 5, 6, or 7 position. In some instances, the asymmetric PEG-lipid has a structure of Formula B-31 A, B-33A, or B-35A. In some instances, the asymmetric PEG-lipid is Compound B-31, B-33, or B-35. In some instances, the asymmetric PEG-lipid is functionalized, for example, PEG-lipids of Formula B-36A and Compounds B-36 or B-37 (bromomaleimide), PEG-lipids of Formula B-38A and Compounds B-38 or B-39 (alkynoic imide), or PEG-lipids of Formula B-40A and Compounds B-40 or B-41 (alkynoic amide). In some embodiments, the LNP further comprises a symmetric PEG-lipid newly disclosed herein. In some embodiments, the functionalized PEG-lipid has been conjugated to a binding moiety so that the LNP is a tLNP.

In certain aspects, the instant disclosure provides a tLNP comprising a PEG-lipid conjugated to a binding moiety wherein the conjugation linkage comprises a reaction product of a thiol in the binding moiety with bromomaleimide or bromomaleimide amide, alkynylamide, or alkynylimide. In some embodiments, a “binding moiety” or “targeting moiety” refers to a protein, polypeptide, oligopeptide, peptide, carbohydrate, nucleic acid, or combination thereof that is capable of specifically binding to a target or multiple targets. A binding domain includes any naturally occurring, synthetic, semi-synthetic, or recombinantly produced binding partner for a biological molecule or another target of interest. In some embodiments, the binding moiety is a polypeptide comprising a binding domain and an N- or C-terminal extension comprising an accessible thiol group. In various embodiments, the binding moiety of the tLNP comprises an antigen binding domain of an antibody, an antigen, a ligand-binding domain of a receptor, or a receptor ligand. In some embodiments, the binding moiety comprising an antigen binding domain of an antibody comprises a complete antibody, an F(ab′)2, an Fab, and Fab′, a minibody, a single-chain Fv (scFv), a diabody, a VH domain, or a nanobody, such as a VHH or single domain antibody. In some embodiments, the receptor ligand is a carbohydrate, for example, a carbohydrate comprising terminal galactose or N-acetylgalactosamine units, which are bound by the asialoglycoprotein receptor. These binding moieties constitute means for targeting. Some embodiments specifically include one or more of these binding moieties. Other embodiments specifically exclude one or more of these binding moieties. In some embodiments, the binding moiety is attached to the PEG-lipid through a thiomaleimide linkage resulting from reaction with the bromomaleimide functionalized PEG-lipid. In some embodiments, the binding moiety is attached to the PEG-lipid through a thioacrylamide linkage resulting from reaction with the alkynoic amide functionalized PEG-lipid. In some embodiments, the binding moiety is attached to the PEG-lipid through a thio-N-acetyl acrylamide linkage resulting from reaction with the alkynoic imide functionalized PEG-lipid.

In some embodiments, a “binding moiety” or “targeting moiety” refers to a protein, polypeptide, oligopeptide, peptide, carbohydrate, nucleic acid, or combination thereof that is capable of specifically binding to a target or multiple targets. A binding domain includes any naturally occurring, synthetic, semi-synthetic, or recombinantly produced binding partner for a biological molecule or another target of interest. Exemplary binding moieties of this disclosure include an antibody, a Fab′, F(ab′)2, Fab, Fv, rIgG, scFv, hcAbs (heavy chain antibodies), a single domain antibody, VHH, VNAR, sdAbs, nanobody, receptor ectodomains or ligand-binding portions thereof, or ligands (e.g., cytokines, chemokines). A “Fab” (fragment antigen binding) is the part of an antibody that binds to antigens and includes the variable region and CH1 of the heavy chain linked to the light chain via an inter-chain disulfide bond. A variety of assays are known for identifying binding moieties of the present disclosure that specifically bind a particular target, including Western blot, ELISA, and Biacore® analysis. A binding moiety, such as a binding moiety comprising immunoglobulin light and heavy chain variable domains (e.g., scFv), can be incorporated into a variety of protein scaffolds or structures as described herein, such as an antibody or an antigen binding fragment thereof, a scFv-Fc fusion protein, or a fusion protein comprising two or more of such immunoglobulin binding domains.

As used herein, “antibody” refers to a protein comprising an immunoglobulin domain having hypervariable regions determining the specificity with which the antibody binds antigen; so-called complementarity determining regions (CDRs). The term antibody can thus refer to intact or whole antibodies as well as antibody fragments and constructs comprising an antigen binding portion of a whole antibody. While the canonical natural antibody has a pair of heavy and light chains, camelids (camels, alpacas, llamas, etc.) produce antibodies with both the canonical structure and antibodies comprising only heavy chains. The variable region of the camelid heavy chain only antibody has a distinct structure with a lengthened CDR3 referred to as VHH or, when produced as a fragment, a nanobody. Antigen binding fragments and constructs of antibodies include F(ab)₂, F(ab), minibodies, Fv, single-chain Fv (scFv), diabodies, and VH. Such elements may be combined to produce bi- and multi-specific reagents, such as BiTEs. The term “monoclonal antibody” arose out of hybridoma technology but is now used to refer to any singular molecular species of antibody regardless of how it was originated or produced. Antibodies can be obtained through immunization, selection from a naïve or immunized library (for example, by phage display), alteration of an isolated antibody-encoding sequence, or any combination thereof. Numerous antibodies that could be used as binding moieties are known in the art. An excellent source of information about antibodies for which an International Non-proprietary Name (INN) has been proposed or recommended, including sequence information, is Wilkinson & Hale, MAbs 14(1):2123299, 2022, including its Supplementary Tables, which is incorporated by reference herein for all that it teaches about individual antibodies and the various antibody formats that can be constructed. U.S. Pat. No. 11,326,182 and especially its Table 9 Cancer, Inflammation and Immune System Antibodies, is a source of sequence and other information for a wide range of antibodies including many that do not have an INN and is incorporated herein by reference for all that it teaches about individual antibodies.

An antibody or other binding moiety (or a fusion protein thereof) “specifically binds” a target if it binds the target with an affinity or Ka (i.e., an equilibrium association constant of a particular binding interaction with units of 1/M) equal to or greater than 10⁵ M⁻¹, while not significantly binding other components present in a test sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Ka of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Ka of up to 10⁸ M⁻¹, up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al., Ann. N.Y. Acad. Sci. 51:660, 1949; and U.S. Pat. Nos. 5,283,173, 5,468,614, or the equivalent).

With respect to any of the LNP aspects, the LNP, including the tLNP, may further comprise one or more of an ionizable cationic lipid, a phospholipid, a sterol, a co-lipid, and a further PEG-lipid, or combinations thereof. In some embodiments, the further PEG-lipid is not functionalized or conjugated. In some embodiments, the herein newly disclosed PEG-lipids serve as the non-functionalized PEG-lipid, the functionalized or conjugated PEG-lipid, or both. As used herein, functionalized PEG-lipid refers to a PEG-lipid in which the PEG moiety has been derivatized with a chemically reactive group that can be used for conjugating a targeting moiety to the PEG-lipid. The functionalized PEG-lipid can be reacted with a binding moiety after the LNP is formed, so that the binding moiety is conjugated to the PEG portion of the lipid. In embodiments comprising both conjugated and unconjugated PEG-lipids, in some the conjugated and unconjugated PEG-lipid are the same and in others the PEG-lipids are different.

With respect to the LNP or the tLNP, in various embodiments, the phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof. Phospholipids contribute to formation of a membrane, whether monolayer or bilayer, surrounding the core of the LNP or tLNP. Additionally, phospholipids such as DSPC, DMPC, DPPC, DAPC impart stability and rigidity to membrane structure. phospholipids such as DOPE impart fusogenicity. Further phospholipids such as DMPG, which attains negative charge at physiologic pH, facilitates charge modulation. Thus, phospholipids constitute means for membrane formation, means for imparting membrane stability and rigidity, means for imparting fusogenicity, and means for charge modulation.

With respect to the LNP or the tLNP, in various embodiments, the sterol is cholesterol or a phytosterol. In further embodiments the phytosterol comprises campesterol, sitosterol, or stigmasterol, or combinations thereof. In preferred embodiments, the cholesterol is not animal-sourced but is obtained by synthesis using a plant sterol as a starting point. LNPs incorporating C-24 alkyl (such as methyl or ethyl) phytosterols have been reported to provide enhanced gene transfection. The length of the alkyl tail, the flexibility of the sterol ring, and polarity related to a retain C-3 —OH group are important to obtaining high transfection efficiency. While β-sitosterol and stigmasterol performed well, vitamin D2, D3 and calcipotriol, (analogs lacking intact body of cholesterol) and betulin, lupeol ursolic acid and olenolic acid (comprising a 5th ring) should be avoided. Sterols serve to fill space between other lipids in the LNP and influence LNP shape. Sterols also control fluidity of lipid compositions, reducing temperature dependence. Thus, sterols such as cholesterol, campesterol, fucosterol, R-sitosterol, and stigmasterol constitute means for controlling LNP shape and fluidity or sterol means for increasing transfection efficiency.

With respect to the LNP or the tLNP, in some embodiments, the co-lipid is absent or comprises an ionizable lipid, anionic or cationic. The co-lipid can be used to adjust any property of the LNP or tLNP such as surface charge, fluidity, rigidity, size, stability, etc. In some embodiments the ionizable lipid is cholesterol hemisuccinate (CHEMS). In some embodiments, the co-lipid is a charged lipid, such as a quaternary ammonium headgroup containing lipid. In some instances, the quaternary ammonium headgroup containing lipid comprises 1,2-dioleoyl-3-trimethylammonium propane (DOTAP), N-(1-(2,3-dioleyloxy)propyl)-N,N,N-trimethylammonium (DOTMA), or 3p-(N-(N′,N′-Dimethylaminoethane)carbamoyl)cholesterol (DC-Chol), or combinations thereof. These compounds are commonly provided as chloride, bromide, mesylate, or tosylate salts.

With respect to the LNP or the tLNP, in some embodiments, the further PEG-lipid (that is, a lipid conjugated to a polyethylene glycol (PEG)) is a C₁₄-C₂₀ lipid such as a C₁₄, C₁₅, C₁₆, C₁₇, C₁₈, C₁₉, or C₂₀ lipid conjugated with a PEG. PEG-lipids with fatty acid chain lengths less than C₁₄ are too rapidly lost from the (t)LNP while those with chain lengths greater than C₂₀ are prone to difficulties with formulation. In some embodiments, the PEG is of 500-5000 Da molecular weight (MW) such as PEG-500, PEG-1000, PEG-1500, PEG-2000, PEG-2500, PEG-3000, PEG-3500, PEG-4000, PEG-4500, and PEG-5000. In some embodiments, the PEG unit has a MW of 2000 Da. In some instances, the MW2000 PEG-lipid comprises DMG-PEG2000 (1,2-dimyristoyl-glycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof. In some embodiments, the glycerol moiety is racemic. Alternatively, optically pure antipodes of the glycerol portion can be employed. Such PEG-lipids constitute old means for preventing aggregation or conjugating a binding moiety and are distinct from the means for preventing aggregation or conjugating a binding moiety related to the PEG-lipids newly disclosed herein.

An LNP that is not a tLNP or a precursor to a tLNP will generally not comprise a functionalized or functionalized/conjugated PEG-lipid. Nonetheless, in some embodiments, such LNP may comprise a mixture of 1) a PEG-lipid newly disclosed herein, and 2) a further PEG-lipid known in the art, such as those listed in the preceding paragraph.

In some embodiments, for LNP comprising both 1) a functionalized or functionalized/conjugated PEG-lipid and 2) PEG-lipid that has not been functionalized, both 1) and 2) are PEG-lipids newly disclosed herein. In some instances, the functionalization is any functionalization newly disclosed herein or otherwise know in the art. In other instances, the functionalization is one of the PEG-lipid functionalizations newly disclosed herein, that is, bromomaleimide or bromomaleimide amide, alkynoic amide, or alkynoic imide. In other embodiments 1) is a PEG-lipid known in the art that has been functionalized as a bromomaleimide or bromomaleimide amide, alkynoic amide, or alkynoic imide and 2) is either a PEG-lipid newly disclosed herein or a previously known PEG-lipid. In still further embodiments, 1) is a PEG-lipid known in the art wherein the functionalization is any functionalization newly disclosed herein or otherwise know in the art and 2) is a herein disclosed PEG-lipid.

### Ionizable Cationic Lipids

With respect to the LNP or the tLNP, in various embodiments, the ionizable cationic lipid comprises a lipid with a measured pKa in the LNP of 6 to 7, facilitating ionization in the endosome. In some embodiments the ionizable cationic lipid has a c-pKa from 8 to 11 and cLogD from 9 to 18 or from 11 to 14. In some embodiments, the ionizable cationic lipids have branched structure to give the lipid a conical rather than cylindrical shape. Suitable ionizable cationic lipids are known to those of skill in the art, including those disclosed in US20130022665, US20180170866, US20160095924, US20120264810, U.S. Pat. Nos. 9,061,063, 9,433,681 9,593,077, 9,642,804 10,196,637, 10,207,010 U.S. Ser. No. 10/383,952, U.S. Pat. Nos. 10,426,737 11,066,355 11,246,993, WO2012170952, WO2021026647, WO2017004143, and WO2017075531 each of which is incorporated by reference for all that it teaches about ionizable cationic lipids. In some preferred embodiments, the ionizable cationic lipid has a structure of Formula 1, Formula 2, or Formula 3, including species or subgenera thereof, as disclosed in U.S. Provisional Application Nos. 63/489,381 filed on Mar. 9, 2023, 63/366,462 filed Jun. 15, 2022, and 63/362,501 filed on Apr. 5, 2022, all entitled Ionizable Cationic Lipids and Lipid Nanoparticles (Atty. Docket No. 146758-8001.US00-02), and PCT application entitled Ionizable Cationic Lipids and Lipid Nanoparticles (Atty. Docket No. 146758-8001.WO00), filed on date even of this application, which are incorporated by reference in their entirety.

In some embodiments, the ionizable cationic lipid has a structure of Formula 1,

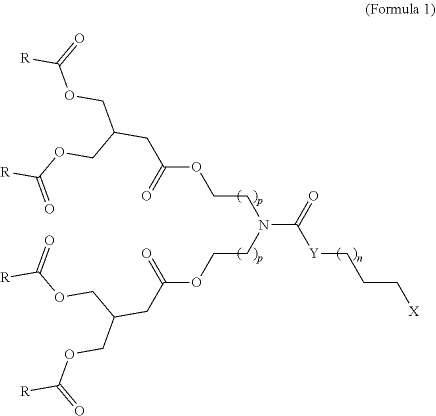

wherein Y is O, NH, N—CH₃, or CH₂,
n is an integer from 0 to 4,
   X is

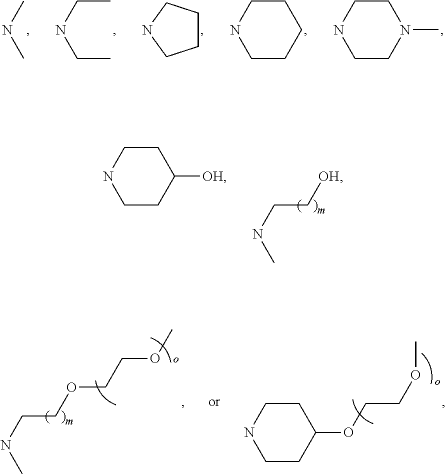

m is an integer from 1 to 3,
o is an integer from 1 to 4,
p is an integer from 1 to 4,
wherein when p=1, each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

Some embodiments specifically include one or more species or subgenera based on specific choices of R, X, Y, m, n, o, p, and/or carbon chain length, structure, or saturation. Other embodiments specifically exclude one or more species or subgenera based on specific choices of R, X, Y, m, n, o, p, and/or carbon chain length, structure, or saturation.

In some embodiments, the ionizable cationic lipid has a structure of Formula 1a

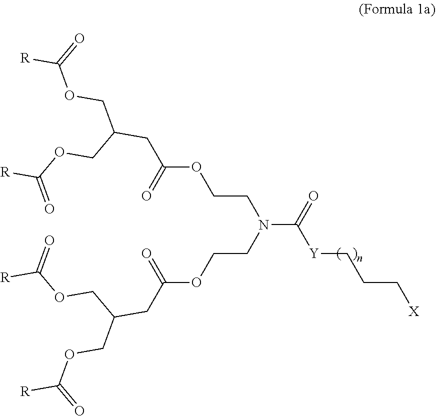

wherein each R is independently C₆ to C₁₆ straight-chain alkyl; C₆ to C₁₆ straight-chain alkenyl; C₆ to C₁₆ branched alkyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
Y is O, NH, N—CH₃, or CH₂,
n is an integer from 0 to 4,
X is

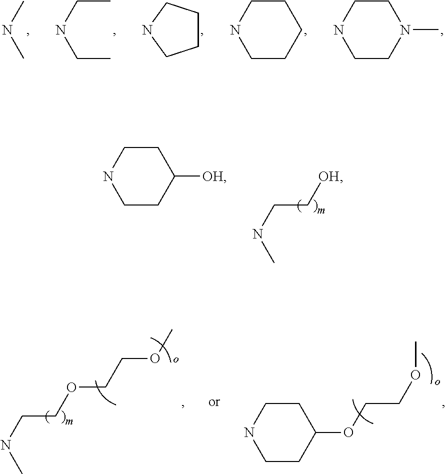

m is an integer from 1 to 3, and
o is an integer from 1 to 4.

In some embodiments, the ionizable cationic lipid having a structure of Formula 1a is:

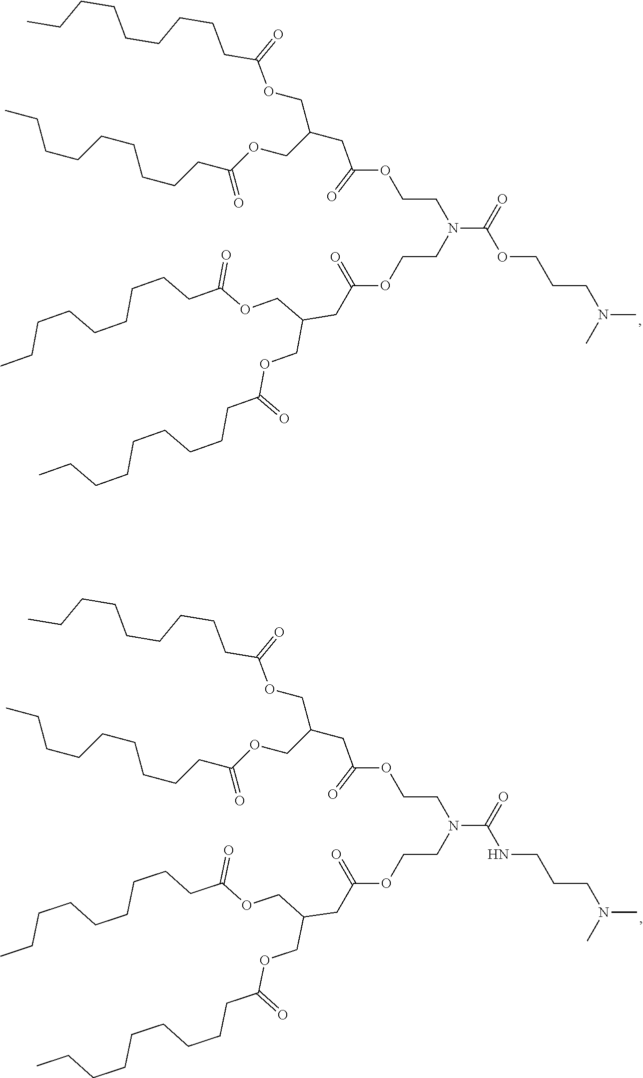

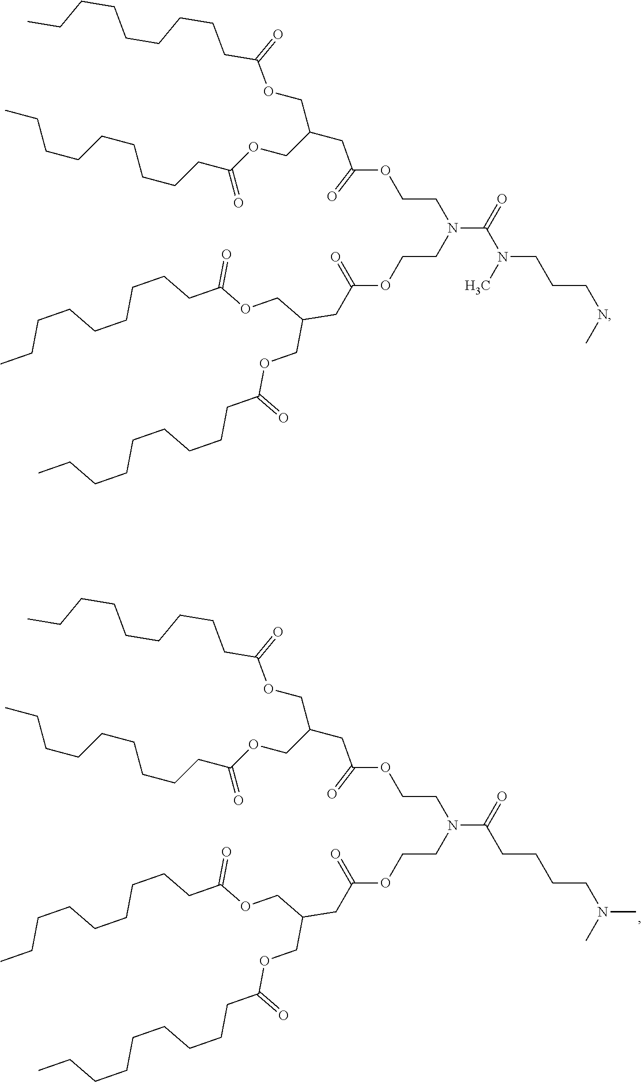

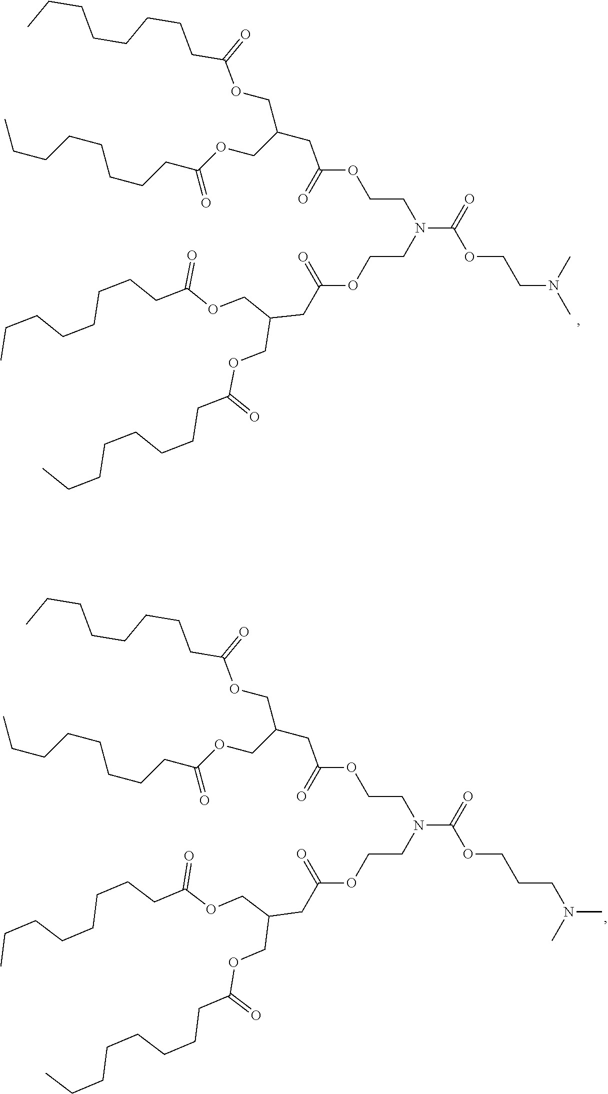

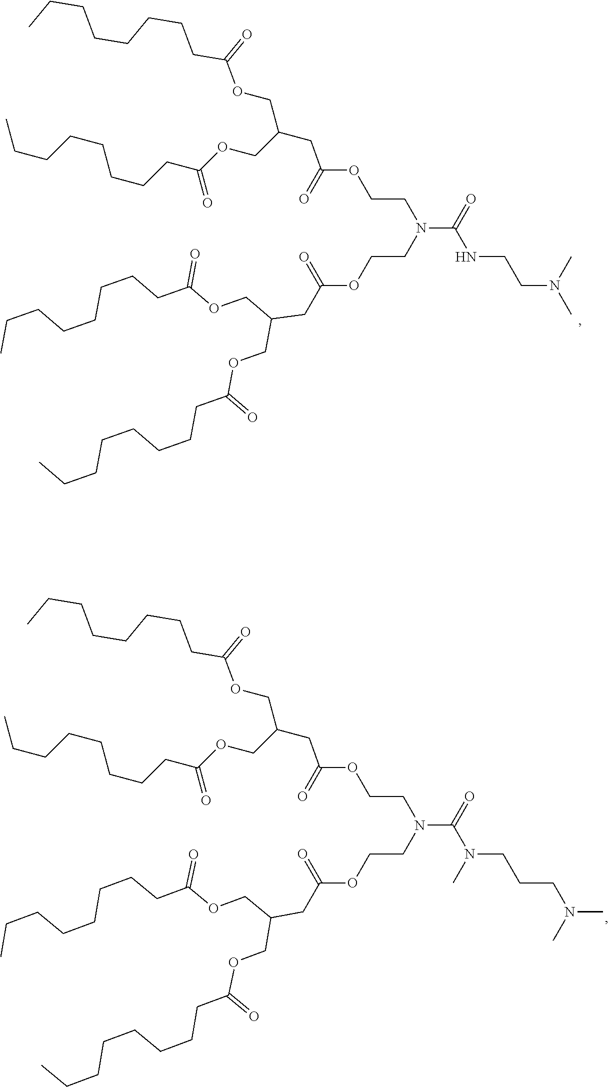

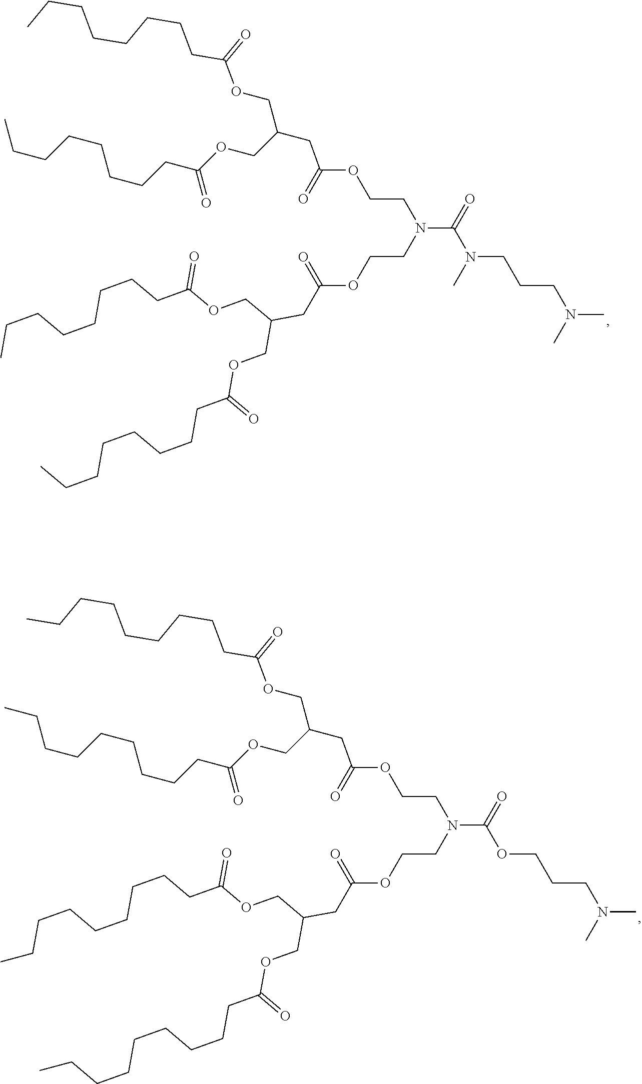

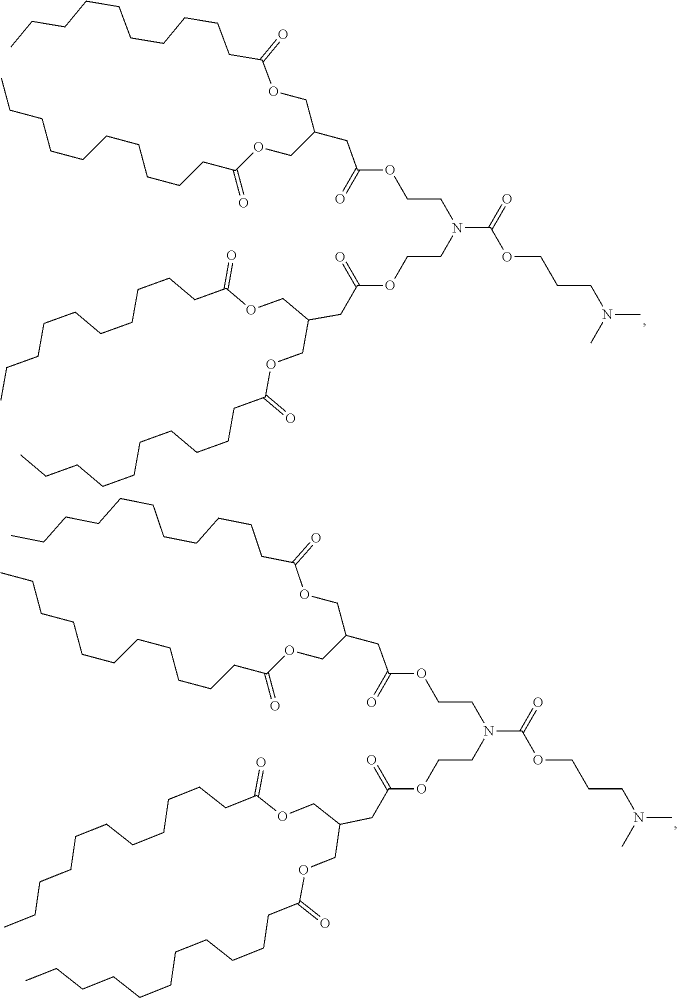

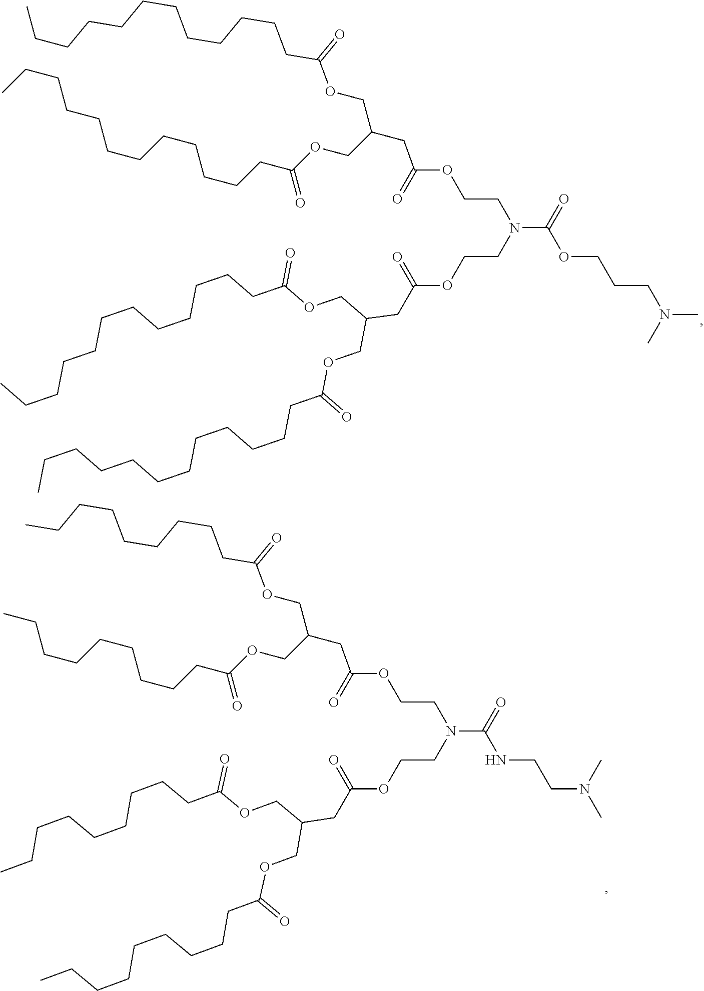

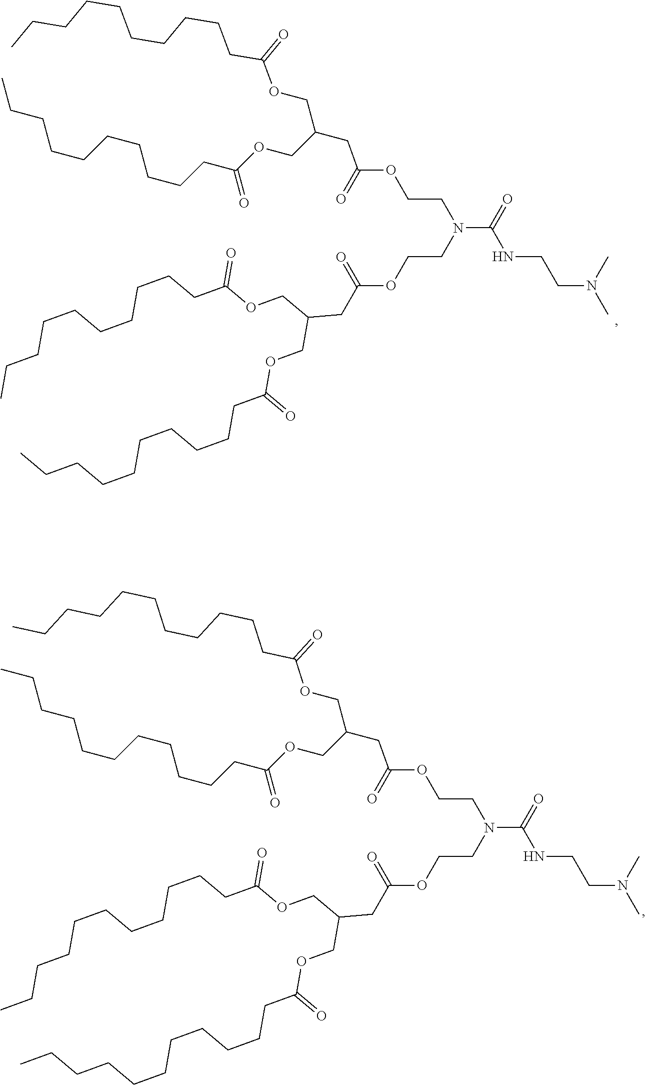

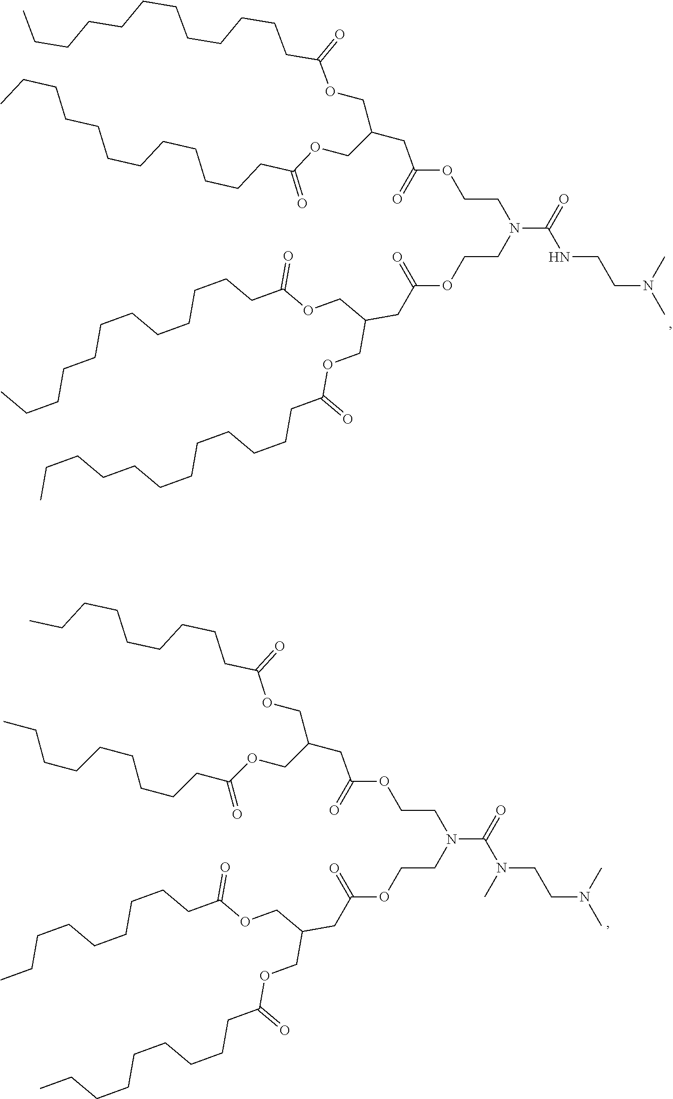

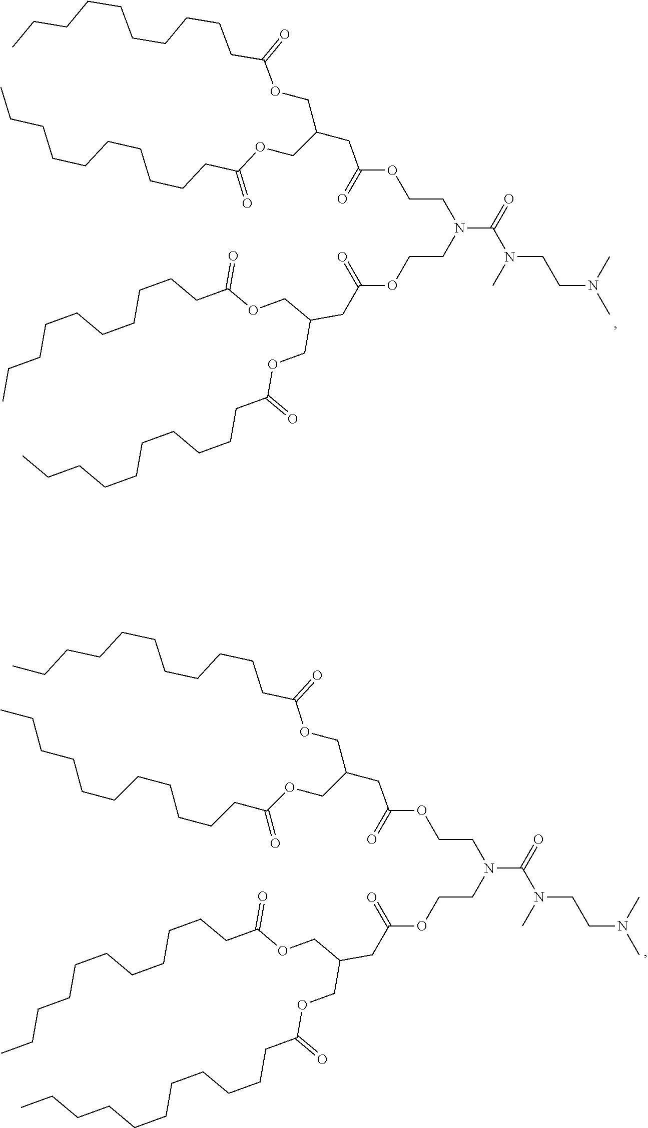

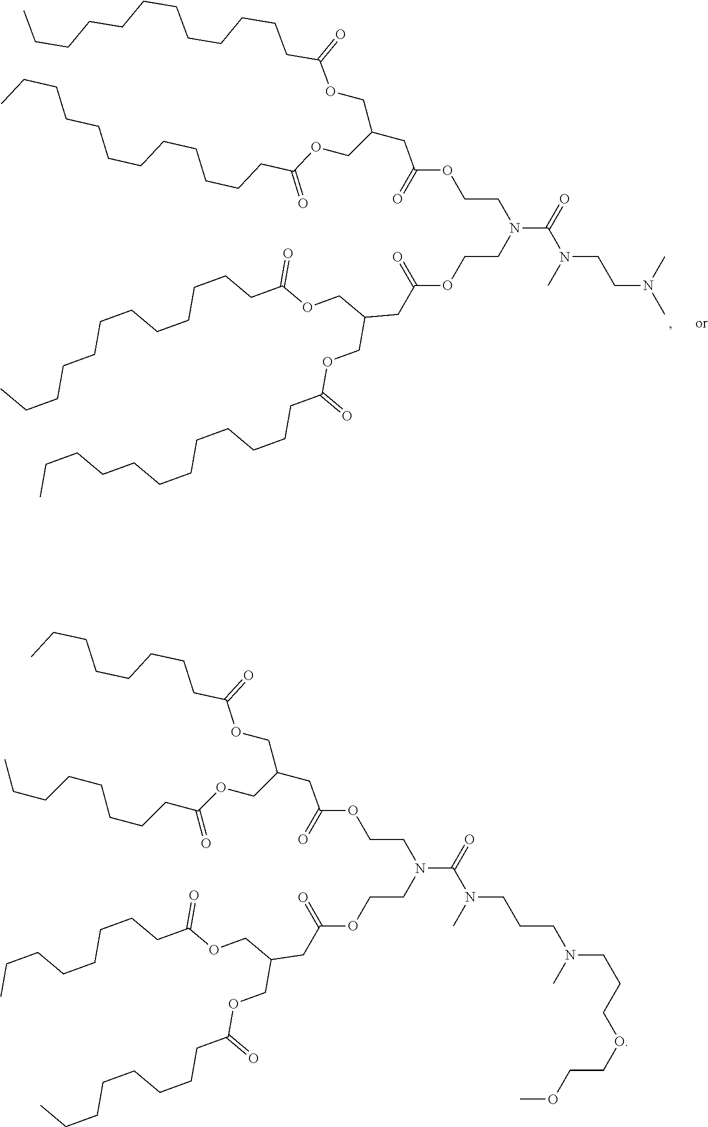

In some embodiments, the ionizable cationic lipid has a structure of Formula 2,

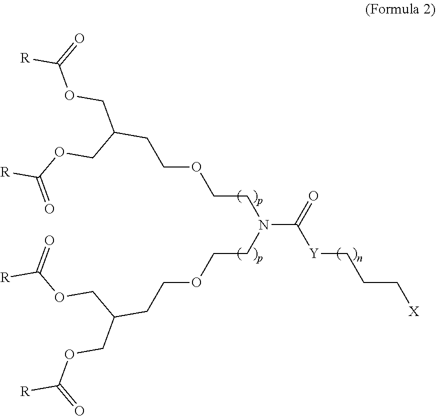

wherein Y is O, NH, N—CH₃, or CH₂,
n is an integer from 0 to 4,
X is

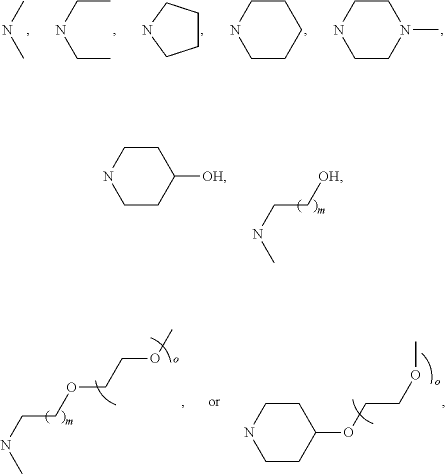

m is an integer from 1 to 3,
o is an integer from 1 to 4,
p is an integer from 1 to 4,
wherein when p=1, each R is independently C₆ to C₁₆ straight-chain alkyl; C₆ to C₁₆ straight-chain alkenyl; C₆ to C₁₆ branched alkyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₆ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; branched C₆ to C₁₂ alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; straight-chain C₆ to C₁₀ alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

Some embodiments include one or more species or subgenera based on specific choices of R, X, Y, m, n, o, p, and/or carbon chain length, structure, or saturation. Other embodiments specifically exclude one or more species or subgenera based on specific choices of R, X, Y, m, n, o, p, and/or carbon chain length, structure, or saturation.

In some embodiments, the ionizable cationic lipid has a structure of Formula 2a

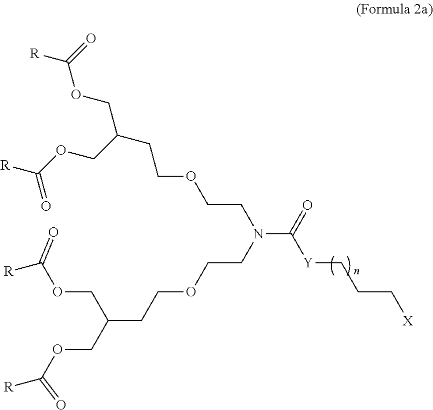

wherein each R is independently C₆ to C₁₆ straight-chain alkyl; C₆ to C₁₆ straight-chain alkenyl; C₆ to C₁₆ branched alkyl; branched C₆ to C₁₆ alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
Y is O, NH, N—CH₃, or CH₂,
n is an integer from 0 to 4,
X is

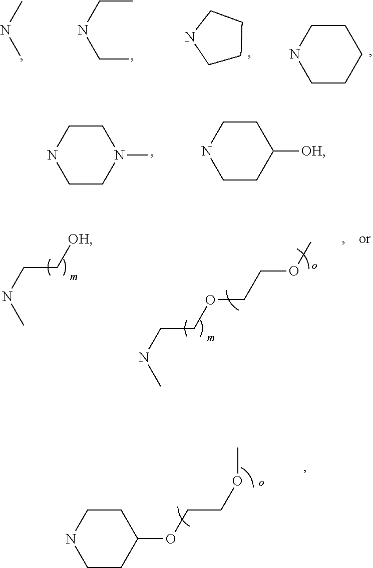

m is an integer from 1 to 3, and
o is an integer from 1 to 4.

In some embodiments, the ionizable cationic lipid has a structure of Formula 3,

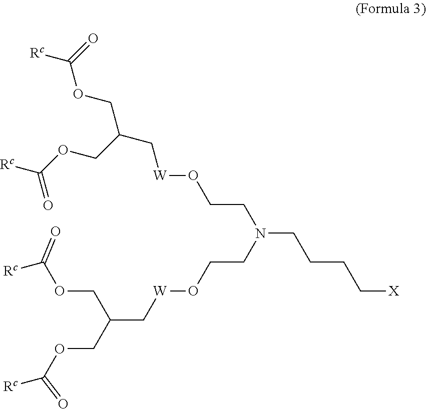

wherein W is C═O or CH₂,
n is an integer from 0 to 4,
X is

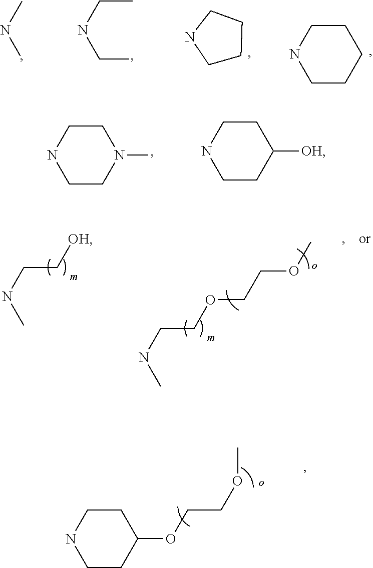

m is an integer from 1 to 3,
o is an integer from 1 to 4,
p is an integer from 1 to 4,
wherein when p is 1, each R^{c} is independently C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
wherein when p=2, each R^{c} is independently C₈ to C₁₆ straight-chain alkyl; C₈ to C₁₆ straight-chain alkenyl; C₈ to C₁₆ branched alkyl; C₈ to C₁₆ branched alkenyl; C₁₁ to C₁₆cycloalkyl-alkyl in which the cycloalkyl is C₃ to C₈cycloalkyl positioned at the either end or within the alkyl chain; or C₁₀ to C₁₈ aryl-alkyl in which the aryl is phenyl or naphthalenyl and is positioned at either end or within the alkyl chain,
wherein when p=3, each R^{c} is independently C₈ to C₁₄ straight-chain alkyl; C₈ to C₁₄ straight-chain alkenyl; C₈ to C₁₄ branched alkyl; C₈ to C₁₄ branched alkenyl; C₁₁ to C₁₄cycloalkyl-alkyl in which the cycloalkyl is C₃ to C₈cycloalkyl positioned at either end or within the alkyl chain; or C₁₀ to C₁₄ aryl-alkyl in which the aryl is phenyl or naphthalenyl and is positioned at the either end or within the alkyl chain, and
wherein when p=4, each R^{c} is independently C₈ to C₁₂ straight-chain alkyl; C₈ to C₁₂ straight-chain alkenyl; C₈ to C₁₂ branched alkyl; C₈ to C₁₂ branched alkenyl; C₁₁ to C₁₂ cycloalkyl-alkyl in which the cycloalkyl is C₃ to C₈ cycloalkyl positioned at either end or within the alkyl; or C₁ to C₁₄ aryl-alky in which the aryl is phenyl or naphthalenyl and is positioned at the either end or within the alkyl chain.

Some embodiments include one or more species or subgenera based on specific choices of R^{c}, W, X, m, n, o, p, and/or carbon chain length, structure, or saturation. Other embodiments specifically exclude one or more species or subgenera based on specific choices of R^{c}, W, X, m, n, o, p, and/or carbon chain length, structure, or saturation.

In some embodiments, the ionizable cationic lipid has a structure of Formula 3a

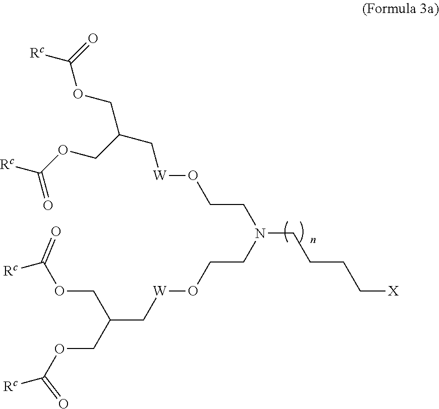

wherein each R^{c} is independently C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈ cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
W is C═O, or CH₂,
n is an integer from 0 to 4,
X is

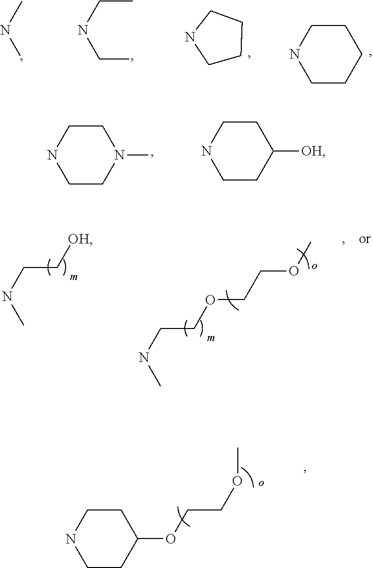

m is an integer from 1 to 3, and
o is an integer from 1 to 4.

With respect to each of the forgoing aspects, in some embodiments, all four R^{c} groups are identical. In other embodiments, the two R^{c} groups stemming from a first branchpoint are identical to each other and the two R^{c} groups from a second branchpoint are identical to each other, but the R^{c} groups stemming from the first branchpoint are different than the R^{c} groups stemming from the second branchpoint.

With respect to each of the forgoing aspects, some embodiments are limited to one, or a subset, of the alternatives for R^{c} W, X, Y, m, n, o, and/or p, as applicable. Other embodiments specifically exclude one, or a subset, of the alternatives for R^{c}, W, X, Y, m, n, o, p, and/or carbon chain length, structure, or saturation, as applicable. Each range of carbon chain length is meant to convey embodiments of all individual lengths and subranges therein.

cLogD is a calculated measure of lipophilicity that takes into account the state of ionization of the molecule and a particular pH, predicting partitioning of the lipid between water and octanol as a function of pH. More specifically, cLogD is calculated at a specified pH based on cLogP and c-pKa. (LogP is the partition coefficient of a molecule between aqueous and lipophilic phases usually considered as octanol and water.) Numerous software packages are available to provide values of cLogD. When higher basicity of the ionizable lipid is desired, it should be balanced by greater lipophilicity as represented by a higher cLogD value. Balance of basicity and lipophilicity is needed for optimal functioning of the LNP for both stability of the particle and release of the cargo (the nucleic acid) upon uptake by a cell. Accordingly, as m, n, or p increases, overall lipophilicity of the ionizable cationic lipid, as represented by cLogD, can be balanced by shorter chain lengths for R. Each of the ionizable cationic lipid species encompassed by Formulas 1-3 have a cLogD in the range of 9 to 18 calculated using ACD Labs Structure Designer v 12.0, cLogP was calculated using ACD Labs Version B; cLogD was calculated at pH 7.4. As different software packages give somewhat different results, this should be accounted for when other software is used, especially near the limits of any ranges stated herein.

A measured pKa in the LNP of 6 to 7 ensures that the ionizable cationic lipid in the LNP will remain neutral in the blood steam and interstitial spaces but ionize after uptake into cells as the endosomes acidify. Upon acidification in the endosomal space the lipid becomes protonated and becomes more strongly associated with the phosphate backbone of the nucleic acid destabilizing the structure of the LNP and promoting release of the nucleic acid from the LNP into the cytoplasm, also referred to as endosomal escape. Thus, the herein disclosed ionizable cationic lipids constitute means for destabilizing LNP structure (when ionized) or means for promoting nucleic acid release or endosomal escape.

### Lipid Nanoparticles

With respect to the LNP or the tLNP, in some embodiments the molar ratio of the lipids is 40 to 60 mol % ionizable cationic lipid: 7 to 30 mol % phospholipid: 20 to 45 mol % sterol: 1 to 30 mol % co-lipid, if present: 0 to 5 mol % PEG-lipid: 0.1 to 5 mol % functionalized PEG-lipid, if present. In some embodiments, the functionalized PEG-lipid is conjugated to a binding moiety.

The LNP or the tLNP further comprises a nucleic acid. In various embodiments the nucleic acid is mRNA, self-replicating RNA, siRNA, miRNA, DNA, a gene editing component (for example, a guide RNA a tracr RNA, sgRNA, an mRNA encoding a gene or base editing protein, a zinc-finger nuclease, a Talen, a CRISPR nuclease, such as Cas9, a DNA molecule to be inserted or serve as a template for repair), and the like, or a combination thereof. In some embodiments the mRNA encodes a chimeric antigen receptor (CAR). In other embodiments the mRNA encodes a gene-editing or base-editing protein. In some embodiments, the nucleic acid is a guide RNA. In some embodiments, the LNP or tLNP comprises both a gene- or base-editing protein-encoding mRNA and one or more guide RNAs. CRISPR nucleases may have altered activity, for example, modifying the nuclease so that it is a nickase instead of making double-strand cuts or so that it binds the sequence specified by the guide RNA but has no enzymatic activity. Base-editing proteins are often fusion proteins comprising a deaminase domain and a sequence-specific DNA binding domain (such as an inactive CRISPR nuclease).

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification relative to the common nucleosides found in natural nucleic acids. For example, over one hundred different nucleoside modifications have been identified in RNA (Rozenski, et al., 1999, The RNA Modification Database: 1999 update. Nucl Acids Res 27:196-197).

In some embodiments, the nucleic acid is an mRNA in which some or all of the uridines have been replaced with pseudouridine, 1-methyl pseudouridine, or another modified nucleoside. In certain embodiments, “pseudouridine” refers, in another embodiment, to m¹acp³Y (1-methyl-3-(3-amino-3-carboxypropyl) pseudouridine. In another embodiment, the term refers to m¹Y (1-methylpseudouridine). In another embodiment, the term refers to Ym (2′-O-methylpseudouridine. In another embodiment, the term refers to m⁵D (5-methyldihydrouridine). In another embodiment, the term refers to m³Y (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

With respect to the LNP or the tLNP, in some embodiments the ratio of total lipid to nucleic acid is 10:1 to 50:1 on a weight basis. In some embodiments, that ratio of total lipid to nucleic acid is 10:1, 20:1, 30:1, or 40:1 to 50:1, or 10:1 to 20:1, 30:1, 40:1 or 50:1, or any range bound by a pair of these ratios.

With respect to the various LNP (including tLNP) aspects, some embodiments specifically exclude one or more of the of the various aspects, embodiments, instances, or species of PEG-lipid, functionalized PEG-lipid, or functionalized/conjugated PEG-lipid. Some embodiments specifically exclude various phospholipids, sterols, co-lipids, and/or further PEG-lipids. Other embodiments specifically include such features.

In certain aspects, the instant disclosure provides a method of making a LNP comprising rapid mixing of an aqueous solution of a nucleic acid and an alcoholic solution of the lipids. The aqueous solution is buffered at pH 3 to 5, for example, with citrate or acetate. In various embodiments, the alcohol can be ethanol or isopropanol or t-butanol. In some embodiments, the rapid mixing is accomplished by pumping the two solutions through a T-junction or an impinging jet mixer. Microfluidic mixing through a staggered herringbone mixer (SHM) or a hydrodynamic mixer (microfluidic hydrodynamic focusing), microfluidic bifurcating mixers, and microfluidic baffle mixers can also be used. After the LNP are formed they are diluted with buffer, for example phosphate, HEPES, or Tris, in a pH range of 6 to 8.5 to reduce the alcohol (ethanol) concentration, The diluted LNP are purified either by dialysis or ultrafiltration or diafiltration using tangential flow filtration (TFF) against a buffer in a pH range of 6 to 8.5 (for example, phosphate, HEPES, or Tris) to remove the alcohol. Alternatively, one can use size exclusion chromatography. Once the alcohol is completely removed the buffer is exchanged with like buffer containing a cryoprotectant (for example, glycerol or a sugar such as sucrose, trehalose, or mannose). The LNP are concentrated to a desired concentrated, followed by 0.2 μm filtration through, for example, a polyethersulfone (PES) filter and filled into glass vials, stoppered, capped, and stored frozen. In alternative embodiments, a lyoprotectant is used and the LNP lyophilized for storage instead of as a frozen liquid. Further methodologies for making LNP can be found, for example, in US20200297634, US20130115274, and WO2017/048770, each of which is incorporated by references for all that they teach about the production of LNP.

In certain aspects, the instant disclosure provides a method of making a tLNP comprising rapid mixing of an aqueous solution of a nucleic acid and an alcoholic solution of the lipids as disclosed for LNP. In some embodiments, the lipid mixture includes functionalized PEG-lipid, for later conjugation to a targeting moiety. As used herein, functionalized PEG-lipid refers to a PEG-lipid in which the PEG moiety has been derivatized with a chemically reactive group (such as, maleimide, NHO ester, Cys, azide, alkyne, and the like) that can be used for conjugating a targeting moiety to the PEG-lipid, and thus, to the LNP comprising the PEG-lipid. In other embodiments, the functionalized PEG-lipid is inserted into and LNP subsequent to initial formation of an LNP from other components. In either type of embodiment, the targeting moiety is conjugated to functionalized PEG-lipid after the functionalized PEG-lipid containing LNP is formed. Protocols for conjugation can be found, for example, in Parhiz et al. J. Controlled Release 291:106-115, 2018, and Tombacz et al., Molecular Therapy 29(11):3293-3304, 2021, each of which is incorporated by reference for all that it teaches about conjugation of PEG-lipids to binding moieties.

After the conjugation the tLNP are purified by dialysis, tangential flow filtration, or size exclusion chromatography, and stored, as disclosed above for LNP.

In other aspects, the instant disclosure provides a method of delivering a nucleic acid into a cell comprising contacting the cell with LNP or tLNP of any of the forgoing aspects. In some embodiments the contacting takes place ex vivo. In some embodiments, the contacting takes place in vivo. In some instances, the in vivo contacting comprises intravenous, intramuscular, subcutaneous, intranodal or intralymphatic administration. In some embodiments, toxicity is confined (or largely confined) to grades of 0 or 1 or two, as discussed above.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein. Although multiple theories have been discussed herein, it is understood that there is no intention to be bound by any theories.

### EXAMPLES

### Example 1: Synthesis and Functionalization of Symmetric Tri-Ester PEG-Lipids on a Dihydroxyacetone Base

Dihydroxy acetone is reacted with (tert-butoxycarbonylmethylene)triphenylphosphorane to give alkenyl-ester IV-1 in 75% yield. Hydrogenation of IV-1 (H₂, Pd/C) then affords t-butyl ester IV-2 in 70% yield (Org. Proc. Research and Development 2011, 15, 515-526). Bis-esterification of IV-2 with stearic acid (EDC-HCl, DMAP) provides IV-3 which is then deblocked to the related acid upon treatment with CF₃CO₂H. A coupling of the acid with PEG-2000 (EDC-HCl, DMAP) affords the tri-ester PEG-lipid Compound B-2 (FIG. 1B).

Similarly, the bis-esterification reaction of IV-2 with the known 4-heptylundecanoic acid (EDC-HCl, DMAP) provides IV-4, which is then de-blocked to the related acid upon treatment with CF₃CO₂H. A coupling of the acid with PEG-2000 (EDC-HCl, DMAP) affords the tri-ester PEG-lipid Compound B-2 (FIG. 1B).

Bromomaleimide functionalization: To functionalize the PEG moiety for stable conjugation with a binding moiety the PEG-lipids Compounds B-1 and B-2 are reacted with bromomaleimide, under Mitsunobu conditions (Ph₃P, DIAD), to provide compounds B-3 and B-4, respectively (FIG. 2B).

Alkynylamide functionalization: The reaction of bis-trimethylsilylacetamide with 3-(trimethylsilyl)-2-propynoyl chloride gives N-acetyl-3-(trimethylsilyl)propiolamide IV-5 and treatment with tetra-n-butyl ammonium fluoride in THF containing HOAc then affords N-acetylpropiolamide IV-6 (FIG. 3A). The coupling of IV-6 with B-1 under Mitsunobu conditions (Ph₃P, DIAD), provides the alkynylamide functionalized PEG-lipid, Compound B-5 (FIG. 3C). Similarly, the coupling of IV-6 with B-2 under Mitsunobu conditions (Ph₃P, DIAD), provides the alkynylamide functionalized PEG-lipid, Compound B-6 (FIG. 3C).

Alkynylimide functionalization: The reaction of t-butyl carbamate (NaH, DMF) with 3-(trimethylsilyl)-2-propynoyl chloride gives imide IV-7 which is desilylated (n-BuN₄NF, THF, HOAc) to provide IV-8 (FIG. 4A). As was described for the syntheses of Compounds B-1 to B-4, B1 and B-2 could be coupled in Mitsunobu fashion (Ph₃P, DIAD) with IV-8 to provide the alkynylimide functionalized PEG-lipid, Compounds B5 and B6, respectively, after BOC removal with acid (FIG. 4C).

### Example 2. Synthesis and Functionalization of Symmetric Di-Ester PEG-Lipids (2-Hydroxymethyl-1,4-Butanediol Scaffold)

2-Hydroxymethyl-1,4-butanediol undergoes an acetal exchange with 4-methoxybenzaldehyde dimethyl acetal, in the presence of p-TsOH in THF (Synthetic Communications 2003, 33, 3897-3905) to provide V-1 (FIG. 5A). Bromination with carbon tetrabromide and triphenyl phosphine in DMF affords bromide V-2 which is coupled with methoxy-PEG-2000 in the presence of NaH in DMF or KOtBu in THF to yield V-3. Hydrolysis with PPTs in MeOH, or hydrogenolysis with H₂ and Pd/C in EtOAc, provides diol V-4. Bis-esterification with 4-heptyl-undecanoic acid in the presence of EDC-HCl and DMAP in CH₂Cl₂ affords target diester B-9. Likewise, bis-esterification of V-4 with stearic acid would lead to bis ester B-10. Note that the PEG moiety in B-9 and B-10 terminates with a methoxyl group.

Alternatively, the reaction of bromide V-2 with 4-methoxybenzyloxy-PEG-2000, in the presence of NaH in DMF or KOtBu in THF affords V-5 (FIG. 5D). Acetal hydrolysis with PPTs in MeOH leads to diol V-6. Bis-esterification of V-6 in the presence of EDC-HCl and DMAP in CH₂Cl₂ affords target diester B-11. Likewise, bis-esterification of V-6 with stearic acid would lead to bis ester B-12. Note that the PEG moiety in B-11 and B-12 terminate in a 4-methoxybenzyloxyl group.

Hydrogenolytic cleavage of the p-methoxybenzyl group of B-11 with H₂ and Pd/C in EtOAc provides alcohol B-13 (FIG. 6B) which can be coupled in Mitsunobu fashion (Ph₃P, DIAD, THF) with bromomaleimide to give B-15. Similarly, the Mitsunobu coupling of B-13 with IV-6 can provide B-17, while the Mitsunobu coupling of B-13 with IV-8 leads to B-19 after BOC removal (TFA, CH₂Cl₂).

Hydrogenolytic cleavage of the p-methoxybenzyl group of B-12 (H₂, Pd/C, EtOAc) provides alcohol B-14 (FIG. 6C) which can be coupled in Mitsunobu fashion (Ph₃P, DIAD, THF) with bromomaleimide to give B-16. Similarly, the Mitsunobu coupling of B-14 with IV-6 can provide B-18, while the Mitsunobu coupling of B-13 with IV-8 leads to B-20 after BOC removal (TFA, CH₂Cl₂).

### Example 3. Synthesis and Functionalization of Symmetric Di-Ester PEG-Lipids (Glycerol Scaffold)

The reaction of the sodium salt of 2,2-dimethyl-1,3-dioxan-5-ol (NaH, DMF) with the mesylate of methoxy-PEG-2000 (VI-1; see Example 4) provides the acetonide VI-2 (FIG. 7B). Acetonide hydrolysis (aq. HCl, MeOH) then affords diol VI-3. Bis-esterification of VI-3 with 4-heptyl-undecanoic acid (EDC-HCl, DMAP, CH₂Cl₂) gives target diester B-21. Likewise, bis-esterification of VI-3 with stearic acid would lead to bis ester B-22. Note that the PEG moiety in B-21 and B-22 terminate in a methoxyl group.

The reaction of the sodium salt of 2,2-dimethyl-1,3-dioxan-5-ol (NaH, DMF) with the mesylate of 4-methoxybenzyloxy-PEG-2000 (VI-4) provides the acetonide VI-5 (FIG. 8C). Acetonide hydrolysis (aq. HCl, MeOH) then affords diol VI-6. Bis-esterification of VI-6 with stearic acid (EDC-HCl, DMAP, CH₂Cl₂) leads to bis ester VI-8. Hydrogenylytic cleavage of the 4-methoxybenzyl group (H₂, Pd/C, EtOAc) provides alcohol B-24 which can be coupled in Mitsunobu fashion (Ph₃P, DIAD, THF) with bromomaleimide to give B-26. Similarly, the Mitsunobu coupling of B-24 with IV-6 can provide B-28, while the Mitsunobu coupling of B-24 with IV-8 leads to B-30 after BOC removal (TFA, CH₂Cl₂).

Bis-esterification of VI-6 with 4-heptyl-undecanoic acid (EDC-HCl, DMAP, CH₂Cl₂) leads to bis ester VI-7 (FIG. 8B). Hydrogenylytic cleavage of the 4-methoxybenzyl group (H₂, Pd/C, EtOAc) provides alcohol B-23 which can be coupled in Mitsunobu fashion (Ph₃P, DIAD, THF) with bromomaleimide to give B-25. Similarly, the Mitsunobu coupling of B-23 with IV-6 can provide B-27, while the Mitsunobu coupling of B-23 with IV-8 leads to B-29 after BOC removal (TFA, CH₂Cl₂).

### Example 4. Synthesis and Functionalization of Asymmetric Di-Ester PEG-Lipids (Glycerol Scaffold)

Methoxy-PEG-2000 is reacted with methanesulfonyl chloride (Et3N, THF) to yield mesylate VI-1 (FIG. 9D). Mesylate VI-1 is then reacted with the sodium salt of (S)-2,2,-dimethyl-dioxolane-4-methanol (NaH, DMF) to give acetonide VII-1. Acetonide hydrolysis with aq. HCl in MeOH produces diol VII-2. Bis-esterification of VII-2 with 4-heptyl-undecanoic acid (EDC-HCl, DMAP, CH₂Cl₂) would give target diester B-31, a PEG-lipid with a glycerol scaffold in which the PEG moiety terminates with a methoxyl group.

The reaction of the sodium salt of PEG-2000 (NaH, DMF) with 4-methoxybenzyl bromide (FIG. 9E) provides VII-3. The reaction of PEG-2000 with 4-methoxybenzyl trichloroacetimidate (THF, CF₃SO₃H) also leads to VII-3. VII-3 can then be converted to the mesylate VII-4 (methanesulfonyl chloride, Et3N, THF). Mesylate VII-4 is then reacted with the sodium salt of (S)-2,2,-dimethyl-dioxolane-4-methanol (NaH, DMF) to give acetonide VII-5. Acetonide hydrolysis with aq. HCl in methanol provides diol, VII-6.

Bis-esterification of VII-6 with 4-heptyl-undecanoic acid (EDC-HCl, DMAP, CH₂Cl₂) gives diester B-33 (FIG. 9F), a PEG-lipid with a glycerol scaffold and a PEG moiety terminating with a 4-methoxybenzyloxyl group. Likewise, bis-esterification of VII-6 with stearic acid (EDC-HCl, DMAP, CH₂Cl₂) gives diester B-32. Hydrogenolytic cleavage (H₂, Pd/C, EtOAC) of the 4-MeO-benzyl group of B-33 affords alcohol B-35. Similar cleavage of the 4-MeO-benzyl group of B-32 gives alcohol B-34.

Alcohol B-34 (FIG. 101B) can then be coupled in Mitsunobu fashion (Ph₃P, DIAD, THF) with bromomaleimide to give B-36. Similarly, the Mitsunobu coupling of B-34 with IV-6 can provide B-38, while the Mitsunobu coupling of B-34 with IV-8 leads to B-40 after BOC removal (TFA, CH₂Cl₂).

Analogously, alcohol B-35 (FIG. 10C) can then be coupled in Mitsunobu fashion (Ph₃P, DIAD, THF) with bromomaleimide to give B-37. Similarly, the Mitsunobu coupling of B-35 with IV-6 can provide B-39, while the Mitsunobu coupling of B-35 with IV-8 leads to B-41 after BOC removal (TFA, CH₂Cl₂).

### Example 5. Synthesis of Symmetric Tri-Ester PEG-Lipids on a Dihydroxyacetone Base with PEG Ethers

Triester IV-3 is deblocked to provide the related acid upon treatment with CF₃CO₂H in CH₂Cl₂. A coupling of the acid with methoxy-PEG-2000 (EDC-HCl, DMAP) affords the tri-ester PEG-lipid Compound B-42. Substituting benzyloxy-PEG-2000 for the methoxy-PEG-2000 used above, leads to B-44, and the use of 4-methoxybenzyl-PEG-2000 (VII-3) in the coupling gives B-46 (FIG. 11B).

Similarly, triester IV-4 is de-blocked (CF₃CO₂H, CH₂Cl₂) to the related acid which can be coupled with methoxy-PEG-2000 (EDC-HCl, DMAP) to afford the tri-ester PEG-lipid Compound B-43. Substituting benzyloxy-PEG-2000 for the methoxy-PEG-2000 used above, leads to B-45, and the use of 4-methoxybenzyloxy-PEG-2000 (VII-3) in the coupling gives B-46 (FIG. 11B).

### Example 6. Synthesis of 4-Methoxybenzyloxy-PEG 2000 (VII-3)

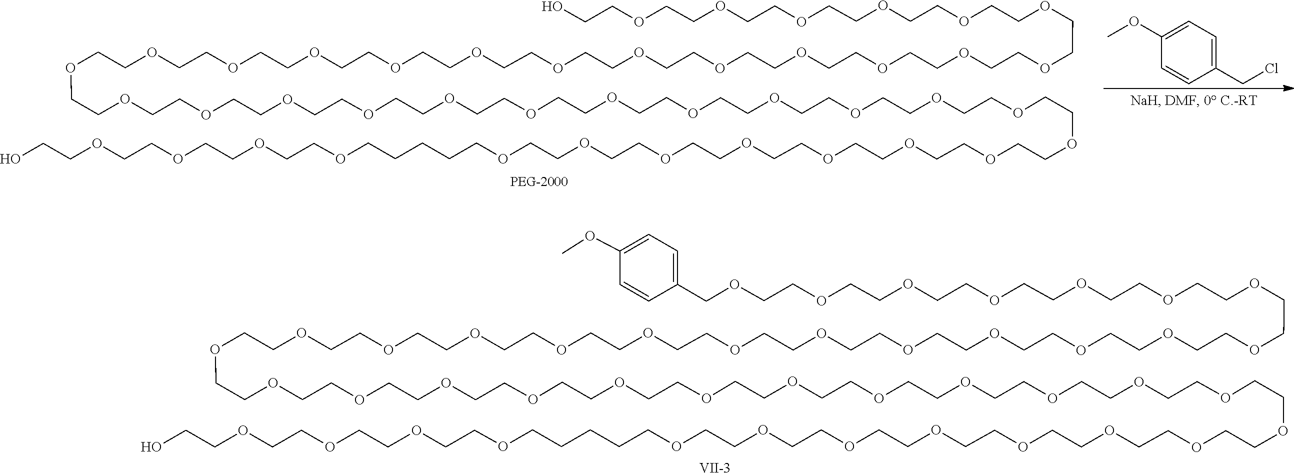

To a solution of PEG-2000 (200 g, 100 mmol), in anhydrous DMF (1.6 L), cooled in an ice-water bath under nitrogen, was added NaH (60% in oil, 4.00 g, 100 mmol) over a period of 10 minutes. The solution was stirred for 30 minutes, then 4-methoxybenzyl chloride (10.40 g, 66 mmol) in DMF (10 mL) was added over a period of 10 minutes. After the addition was complete, the mixture was warmed to room temperature and the reaction was quenched by the addition of saturated aq. NH₄Cl (1.5 L). The aqueous layer was saturated with salt and extracted with CH₂Cl₂ (2×0.5 L). The combined organic phases were washed with brine (2×0.5 L), dried over Na₂SO₄, filtered, and concentrated in vacuo to give crude 1 as a pale-yellow semi-solid. Crude VII-3 was purified by Flash-Prep-HPLC (C₁₈ column, gradient CH₃CN/H₂O 100:0 to 55:45). Fractions containing VII-3 were pooled and concentrated (lyophilization) to give VII-3 (33.10 g, 15.60 mmol, 23.7% based upon 4-MeO-benzyl chloride) as a white solid.

¹H-NMR (300 MHz, CDCl₃): δ 7.27 (d, J=8.70 Hz, 2H), 6.87 (d, J=8.70 Hz, 2H), 3.84-3.90 (3H), 3.80 (s, 3H), 3.55-3.66 (177H), 2.66 (m, 4H); HRMS: Calcd. for C₉₈H₁₉₀O₄₇+Na 2142.24, Found 2143.23.

### Example 7. Synthesis of 4-Methoxybenzyloxy-PEG 2000 Methanesulfonate (VI-4)

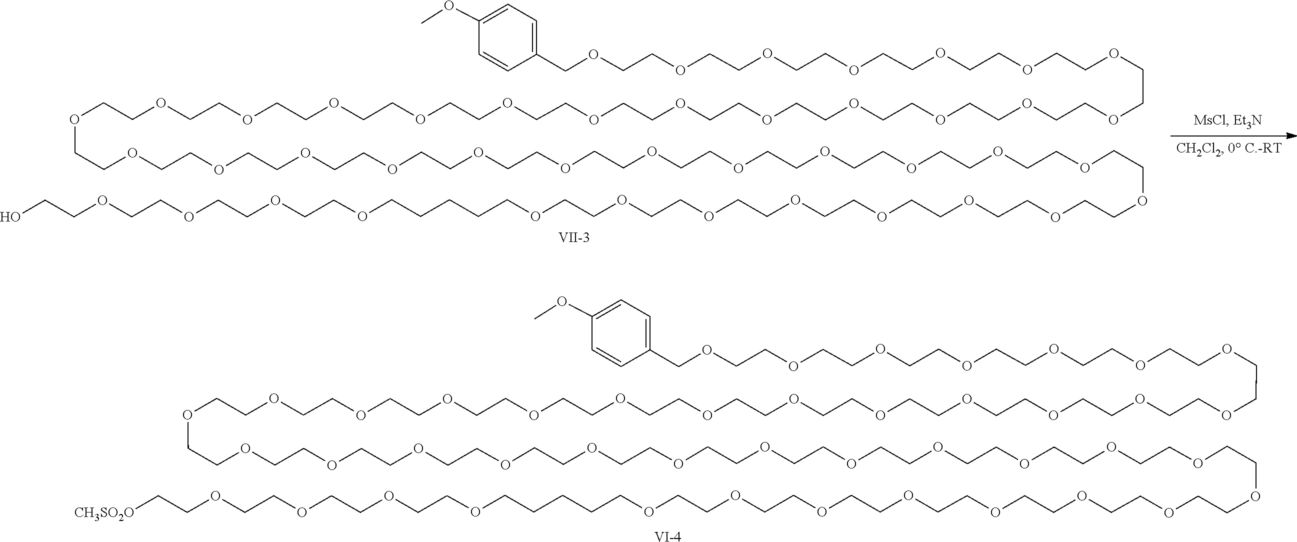

To a solution of VII-3 (28.0 g, 13.2 mmol) in CH₂Cl₂ (280 mL), cooled in an ice-water bath under nitrogen, was added in order: Et3N (2.00 g, 20 mmol) and methanesulfonyl chloride (MsCl, 1.80 g, 15.7 mmol). The mixture was allowed to stir for 1 hour after the additions were complete, then the reaction was quenched by the addition of water (100 mL). The organic phase was separated, the aq. phase was extracted with CH₂Cl₂ (3×100 mL), and the combined organic phases were dried over Na₂SO₄. Filtration and concentration in vacuo afforded VI-4 (26.00 g, 88% purity by HPLC, 11.60 mmol, 88%) as an off-white solid which was carried forward without further purification.

¹H-NMR (300 MHz, CDCl₃): δ 7.26 (d, J=8.70 Hz, 2H), 6.87 (d, J=8.70 Hz, 2H), 4.49 (s, 2H), 4.38 (m, 2H), 3.30-3.90 (181H), 3.09 (s, 3H).

### Example 8. Synthesis of 4-Methoxybenzyloxy-PEG 2000 2,2-Dimethyl-1,3-dioxane-5-ol Ether (VI-5)

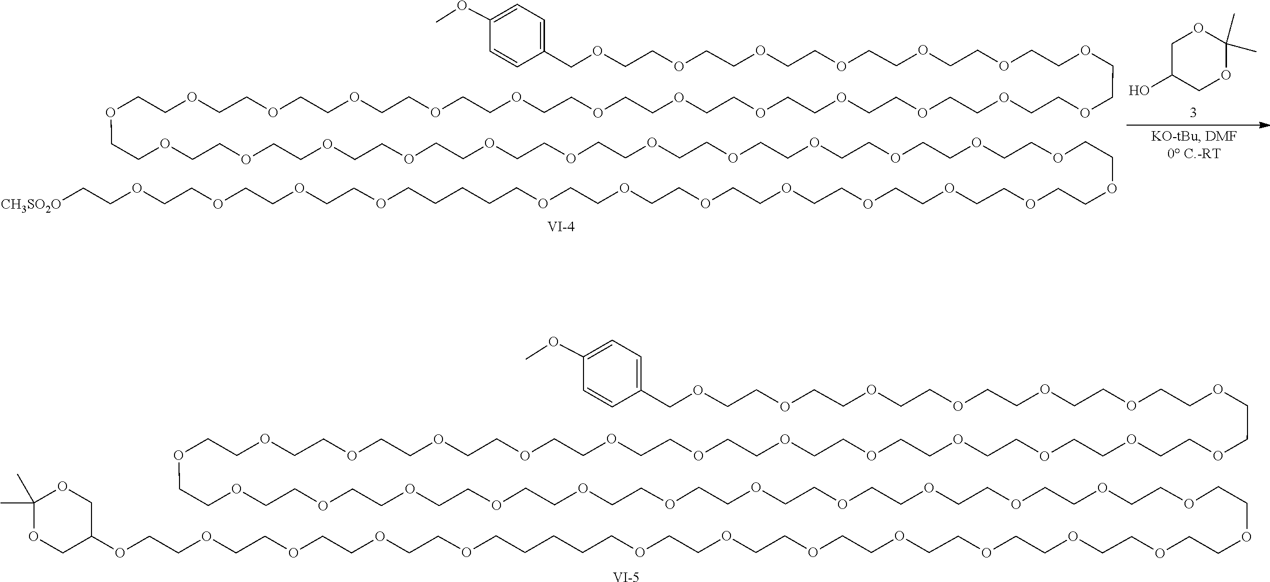

To a solution of 2,2-dimethyl-1,3-dioxan-5-ol 3 (2.39 g, 18.00 mmol, (prepared as described in: Synthesis 1998, 879-882) in anhydrous DMF (24 mL), cooled in an ice-water bath under nitrogen, was added solid KO-tBu (2.70 g, 24.0 mmol) in one portion. The mixture was stirred for 30 minutes after the addition, then mesylate VI-4 (26.50 g, 88% pure, 10.60 mmol) in anhydrous DMF (500 mL) was added over a period of 30 minutes. The mixture was then warmed to room temperature and was stirred for 4 hours. The mixture was cast into saturated aq. NH₄Cl (750 mL) and was extracted with CH₂Cl₂ (3×375 mL). The combined organic phases were washed with brine (5×500 mL), dried over Na₂SO₄, filtered, and concentrated in vacuo to furnish crude VI-5 (30.00 g) as a light-yellow solid. Crude VI-5 was taken without any further purification.

¹H-NMR (300 MHz, CDCl₃): δ 7.27 (d, J=8.70 Hz, 2H), 6.87 (d, J=8.70 Hz, 2H), 4.49 (s, 2H), 3.32-4.00 (183H), 1.43 (s, 3H), 1.39 (s, 3H).

### Example 9. Synthesis of 4-Methoxybenzyloxy-PEG 2000-propane-1,3-diol-2-ether (VI-6)

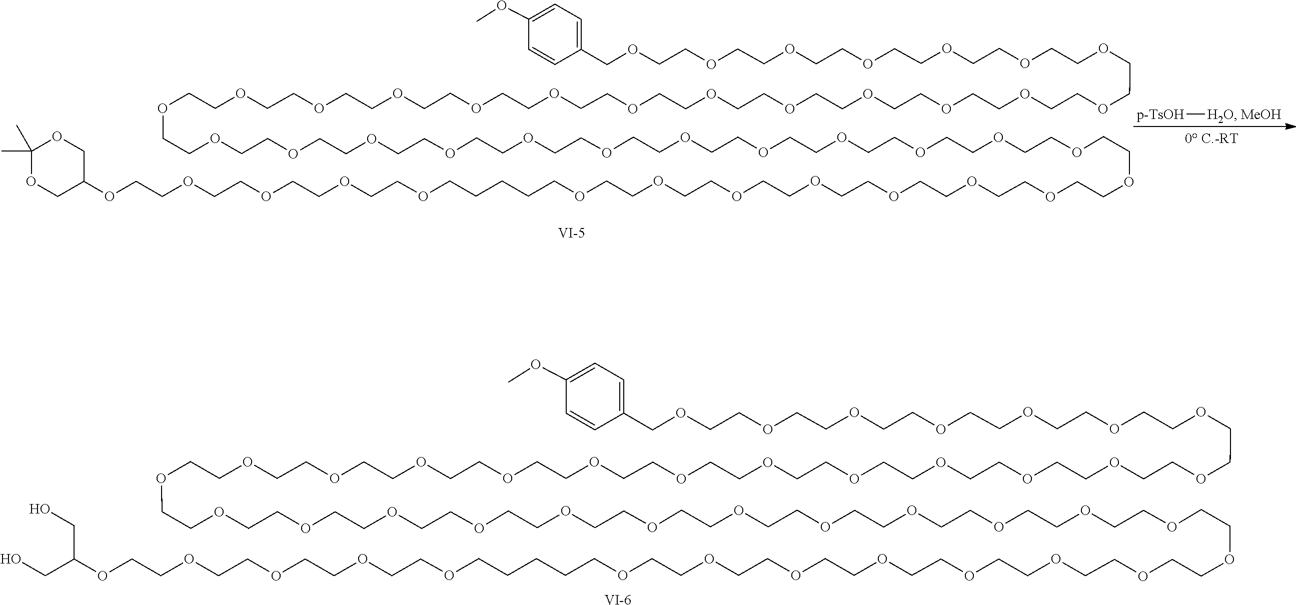

To a solution of acetonide VI-5 (30.0 g, crude) in methanol (MeOH, 600 mL) was added p-TsOH—H₂O (9.07 g, 47.7 mmol). The mixture was stirred for 30 minutes in the ice-water bath then was warmed to room temperature and stirring was continued for an additional 3 hours. The solvent was removed in vacuo and the crude VI-6 was purified Flash-Prep-HPLC (C₁₈ column, gradient CH₃CN/H₂O-0.1% NH₄HCO₃ 100:0 to 55:45). Fractions containing VI-6 were pooled and concentrated (lyophilization) to give VI-6 (19.00 g, 8.66 mmol, 66% from VII-3) as a light-yellow solid.

¹H-NMR (300 MHz, CDCl₃): δ 7.27 (d, J=8.70 Hz, 2H), 6.87 (d, J=8.70 Hz, 2H), 4.49 (s, 2H), 3.40-3.90 (188H).

### Example 10. Synthesis of VI-8

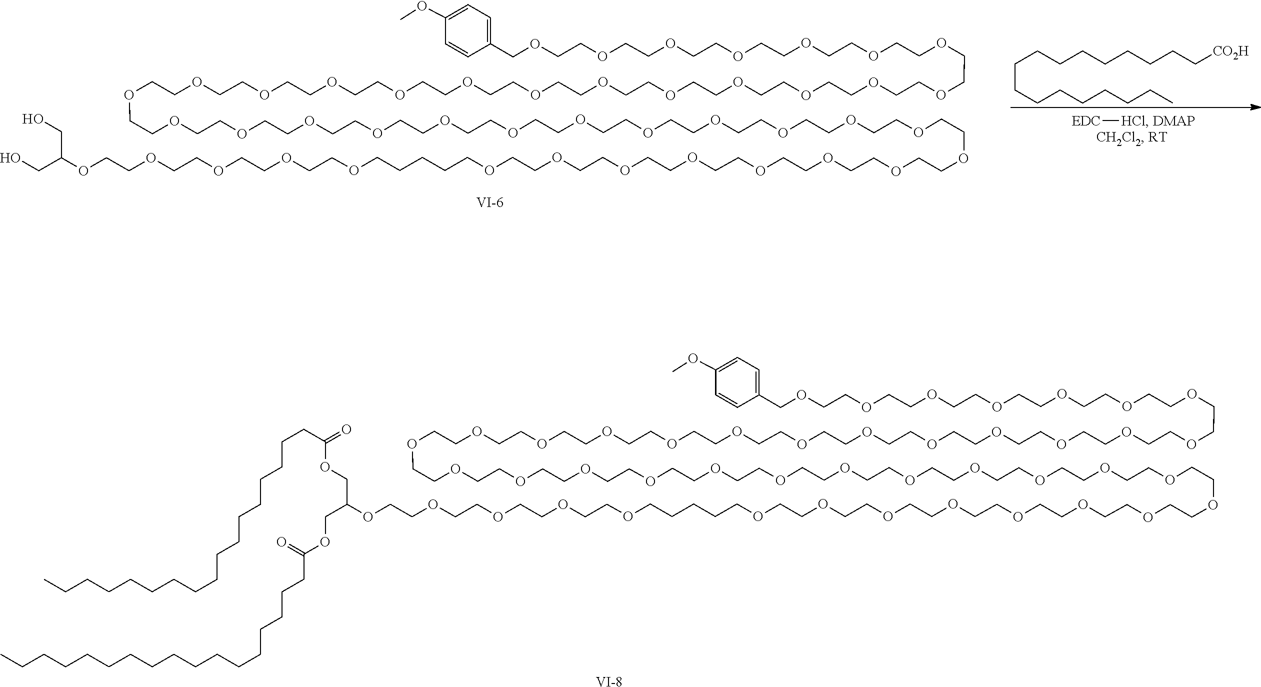

To a solution of diol VI-6 (10.00 g, 4.55 mmol) in CH₂Cl₂ (500 mL), cooled in an ice-water bath under nitrogen, was added in order: stearic acid (2.82 g, 9.91 mmol), 4-dimethylamino-pyridine (DMAP, 0.551 g, 4.51 mmol). The mixture was stirred for 10 minutes, then EDC-HCl (2.18 g, 11.37 mmol) was added in portions over 5 minutes. The was warmed to room temperature and was stirred for 16 hours, then was cast into CH₂Cl₂ (500 mL) and washed in order with 5% aq. NaHCO₃ (500 mL), 5% aq. citric acid (500 mL), and brine (500 mL). The organic phase was dried over Na₂SO₄, filtered, and concentrated in vacuo to give crude VI-8 as a pale-yellow semi solid. Crude VI-8 was purified Flash-Prep-HPLC (C₁₈ column, gradient CH₃CN/H₂O-0.04% TFA 35:65 to 90:10). Fractions containing VI-8 were pooled, the pH was adjusted to 7-8 with 5% aq. ammonium hydroxide, and concentrated. The mixture was diluted with water (50 mL), extracted with CH₂Cl₂ (3×50 mL), and the combined organic phases were dried over Na₂SO₄. Filtration, concentration in vacuo, dissolution in CH₃CH/H₂O (90:10), and lyophilization, gave VI-8 as a white solid.

¹H-NMR (300 MHz, CDCl₃): δ 7.27 (d, J=8.70 Hz, 2H), 6.87 (d, J=8.70 Hz, 2H), 4.50 (s, 2H), 4.17 (m, 4H), 3.35-3.90 (183H), 2.31 (t, J=7.20 Hz, 4H), 1.60 t, J=7.20 Hz, 4H), 1.15-1.37 (56H), 0.87 (m, 6H); HRMS: Calcd. for C₁₃₇H₂₆₄O₅₁+Na⁺2748.800, Found 2748.806.

### Example 11. Synthesis of Dimethyl 2,2-diheptylmalonate (6)

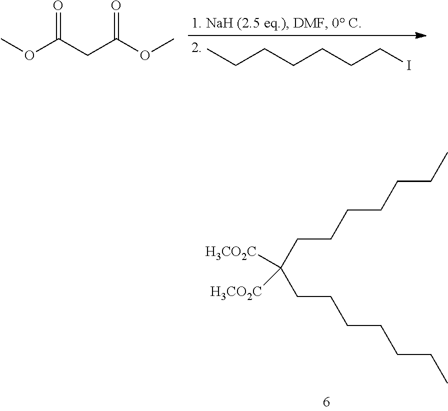

To NaH (60% in oil, 32.68 g, 0.917 mol) in anhydrous DMF (165 mL), cooled in an ice-water bath under nitrogen, was added methyl malonate (46.73 g, 0.353 mol) in DMF (115 mL) over a period of 1 hour. The mixture was stirred for 30 minutes after the addition was complete, then heptyl iodide (200 g, 0.884 mol) in DMF (500 mL) was added over 60 minutes. The mixture was stirred for 3 hours after the addition was complete, then the reaction was quenched by the careful addition of water/ice (1.2 L). The mixture was extracted with ethyl acetate (2×3.0 L), the combined organic phases were washed with water (2×1.0 L), brine (2.1 L), and dried over Na₂SO₄. Filtration and concentration in vacuo gave crude 6 which was dissolved in CH₂Cl₂ (1.0 L,) and silica gel (300 g, type ZCX-2, 100-200 mesh) was added. The solvent was removed in vacuo and the silica gel, impregnated with crude product, was placed atop a gravity column of silica gel (1500 g, type ZCX-2, 100-200 mesh) packed with n-heptane and eluted with a gradient of n-heptane/ethyl acetate from 100:0 to 97:3. Fractions containing 6 were pooled and concentrated in vacuo to provide 6 (82.0 g, 0.249 mol, 71%) as a pale-yellow oil.

¹H-NMR (300 MHz, CDCl₃): δ 3.72 (s, 6H), 1.87 (m, 4H), 1.03-1.39 (20H), 0.89 (t, J=6.90 Hz, 6H).

### Example 12. Synthesis of Methyl 2-heptylnonanoate (7)

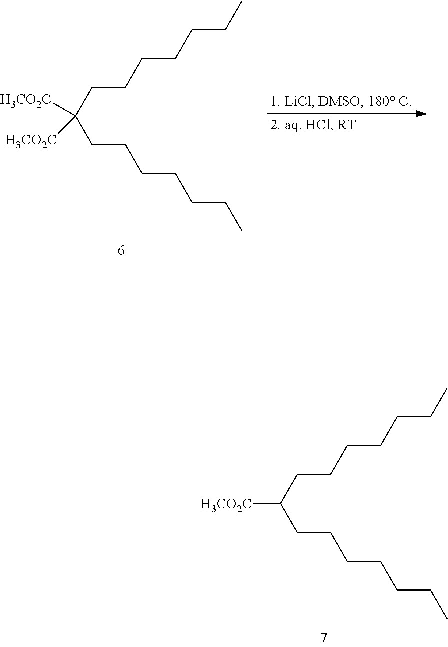 [SMILES: CCCCCCCC(CCCCCCC)(C(=O)OC)C(=O)OC ; CCCCCCCC(CCCCCCC)C(=O)OC]

To a solution of 6 (80.00 g, 0.244 mol), in dry DMSO (400 mL) under nitrogen was added anhydrous LiCl (31.04 g, 0.732 mol). The mixture was warmed to 180° C. and was attired for 16 hours. The mixture was then cooled to room temperature and the reaction was quenched by adding ice-cold 1 N aq. HCl (500 mL). The resulting mixture was extracted with ethyl acetate (2×700 mL). The combined organic phases were washed with water (2×400 mL), brine (400 mL), and dried (Na₂SO₄). Filtration and concentration in vacuo gave crude 7 which was dissolved in ethyl acetate (500 mL), and silica gel (160 g, type ZCX-2, 100-200 mesh) was added. The solvent was removed in vacuo and the silica gel, impregnated with crude product, was placed atop a gravity column of silica gel (800 g, type ZCX-2, 100-200 mesh) packed with n-heptane and eluted with a gradient of n-heptane/ethyl acetate from 100:0 to 90:10. Fractions containing 7 were pooled and concentrated in vacuo to provide 7 (45.0 g, 0.166 mol, 68%) as a colorless oil.

¹H-NMR (300 MHz, CDCl₃): δ 3.69 (s, 3H), 2.33 (m, 1H), 1.61 (m, 2H), 1.45 (m, 2H), 1.25-1.42 (20H), 0.89 (m, 6H).

### Example 13. Synthesis of 2-heptylnonan-1-ol (8)

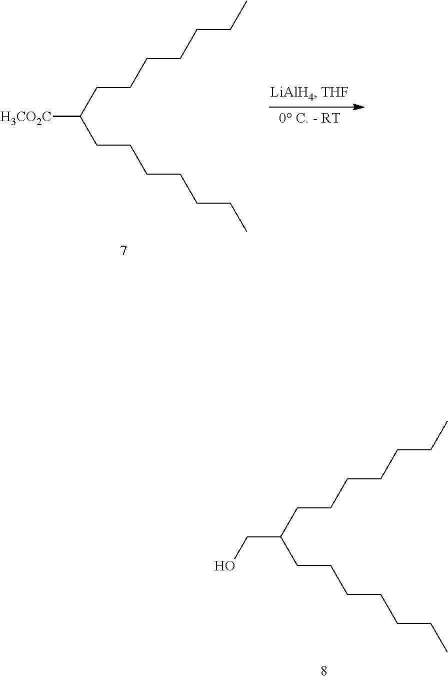 [SMILES: CCCCCCCC(CCCCCCC)C(=O)OC ; CCCCCCCC(CO)CCCCCCC]

To a solution of 7 (45.00 g, 0.166 mol) in anhydrous THF (900 mL), cooled in an ice-water bath under nitrogen, was added LiAlH₄ (12.65 g, 0.333 mol) in portions over 30 minutes. The mixture was stirred for 30 minutes then was warmed to room temperature and was allowed to stir for an additional 2 hours. The reaction was carefully quenched by the addition of ice/water (12 mL, followed by 15% aq. NaOH (12 mL), and ice-water (34 mL). The solids were removed by filtration and the solution was concentrated in vacuo and the residue was dissolved with CH₂Cl₂ (90 mL). To this solution of crude 8 was added silica gel (90 g, type ZCX-2, 100-200 mesh) was added. The solvent was removed in vacuo and the silica gel, impregnated with crude product, was placed atop a gravity column of silica gel (450 g, type ZCX-2, 100-200 mesh) packed with n-heptane and eluted with a gradient of n-heptane/ethyl acetate from 100:0 to 95:5. Fractions containing 8 were pooled and concentrated in vacuo to provide 8 (38.00 g, 0.157 mol, 94%) as a colorless oil.

¹H-NMR (300 MHz, CDCl₃): δ3.56 (m, 2H), 1.20-1.55 (25H), 0.90 (m, 6H).

### Example 14. Synthesis of 2-Heptylnonanal (9)

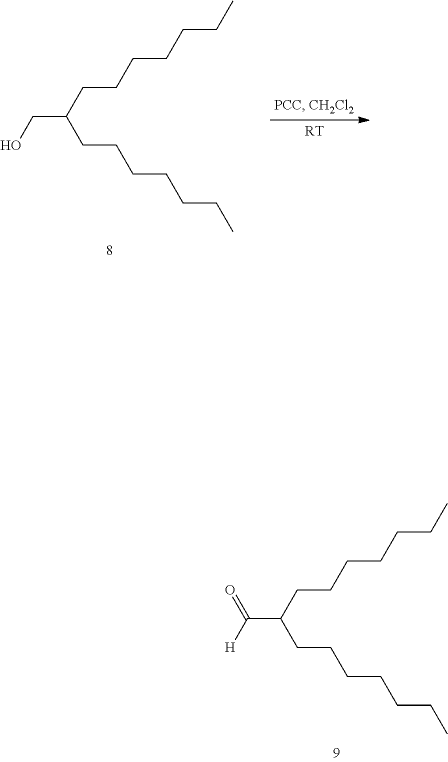 [SMILES: CCCCCCCC(CO)CCCCCCC ; "RT" ; CCCCCCCC(C=O)CCCCCCC]

To a solution of 8 (38.00 g, 0.157 mol), in CH₂Cl₂ (750 mL) under nitrogen at room temperature, was added pyridinium chlorochromate (PCC, 50.76 g, 0.235 mol) in portions over a period of 30 minutes. The mixture was stirred for 2 hours after the addition was complete, then diethyl ether (1.0 L) was added and stirring was continued for 10 minutes. The resulting precipitate was removed by filtration through a Celite pad, the filter cake was rinsed with diethyl ether (250 mL) and the solvent was removed in vacuo. The residue was dissolved in CH₂Cl₂ (250 mL) and added silica gel (76 g, type ZCX-2, 100-200 mesh) was added. The solvent was removed in vacuo and the silica gel, impregnated with crude product, was placed atop a gravity column of silica gel (400 g, type ZCX-2, 100-200 mesh) packed with n-heptane and eluted with a gradient of n-heptane/ethyl acetate from 100:0 to 95:5. Fractions containing 9 were pooled and concentrated in vacuo to provide 9 (18.20 g, 76 mmol, 48%.).

¹H-NMR (300 MHz, CDCl₃): δ 9.55 (d, J=3.30 Hz, 1H), 2.21 (m, 1H), 1.20-1.60 (24H), 0.90 (m, 6H); LCMS: Calcd. for C₁₆H₃₂O+H+241.25. Found: 241.20.

### Example 15. Synthesis of (E)-4-Heptylundec-2-enoic acid (10)

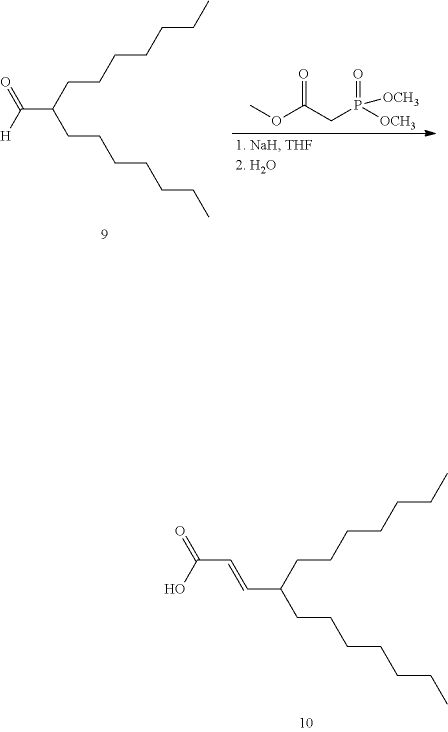 [SMILES: CCCCCCCC(C=O)CCCCCCC ; COC(=O)CP(=O)(OC)OC ; CCCCCCCC(/C=C/C(=O)O)CCCCCCC]

To a solution of trimethyl phosphonoacetate (17.97 g, 98.7 mmol) in anhydrous THF (900 mL), cooled in an ice-water bath under nitrogen, was added NaH (60% in oil, 6.58 g, 0.164 mol) in portions over 15 minutes. The mixture was allowed to stir for 0.5 hours, then a solution of 9 (80% pure, 15.80 g, 52.6 mmol) in THF (75 mL) was added over a period of 30 minutes. The mixture was warmed to room temperature and was stirred for 16 hours. The reaction was quenched by the careful addition of ice-water (300 mL) and the solution was extracted with ethyl acetate (3×300 mL). The combined organic phases were dried over Na₂SO₄, filtered, and concentrated in vacuo. The residue was dissolved in ethyl acetate (150 mL) and silica gel (36 g, type ZCX-2, 100-200 mesh) was added. The solvent was removed in vacuo and the silica gel, impregnated with crude product, was placed atop a gravity column of silica gel (360 g, type ZCX-2, 100-200 mesh) packed with n-heptane and eluted with a gradient of n-heptane/ethyl acetate from 100:0 to 95:5. Fractions containing 10 were pooled and concentrated in vacuo to provide 10 (6.75 g, 23.9 mmol, 45%.).

¹H-NMR (300 MHz, CDCl₃): δ 6.87 (dd, J=15.60 Hz, 3.10 Hz, 1H), 5.80 (d, J=15.60 Hz, 1H), 2.13 (m, 1H), 1.15-1.62 (24H), 0.90 (m, 6H); LCMS: Calcd. for C₁₈H₃₄O₂−H+281.25. Found: 281.20.

### Example 16. Synthesis of 4-Heptylundecanoic acid (11)

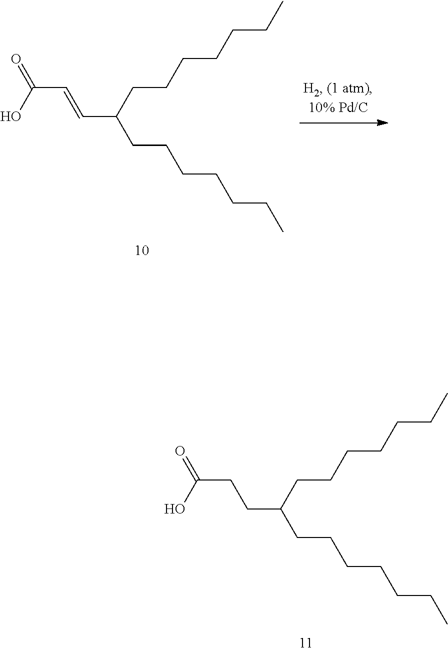 [SMILES: CCCCCCCC(/C=C/C(=O)O)CCCCCCC ; CCCCCCCC(CCCCCCC)CCC(=O)O]

To a solution of 10 (10.80 g, 38.23 mmol) in THF (100 mL) and MeOH (100 mL), at room temperature under nitrogen, was added 10% Pd/C (1.10 g). The flask was evacuated and flushed with nitrogen (3×) then the mixture was placed under a balloon of hydrogen. The mixture was stirred for 2 hours, then the catalyst was removed by filtration through a pad of Celite. The filter cake was rinsed with THF/MeOH (50:50, 100 mL) and the combined filtrates were concentrated in vacuo. The crude 11 was purified by Flash-Prep-HPLC (IntelFlash-1, C₁₈ column, CH₃CN/H₂O with 0.05% TFA, gradient from 60% CH₃CN to 95% CH₃CN). Fractions containing 11 were pooled and concentrated in vacuo to give 11 (8.20 g, 28.82 mmol, 74%) as a pale-yellow oil.

¹H-NMR (300 MHz, CDCl₃): δ 2.33 (m, 2H), 1.60 (m, 2H), 1.18-1.40 (25H), 0.90 (m, 6H); LCMS: Calcd. for C₁₈H₃₆O₂−H+283.26. Found: 283.30.

### Example 17. Synthesis of VI-7

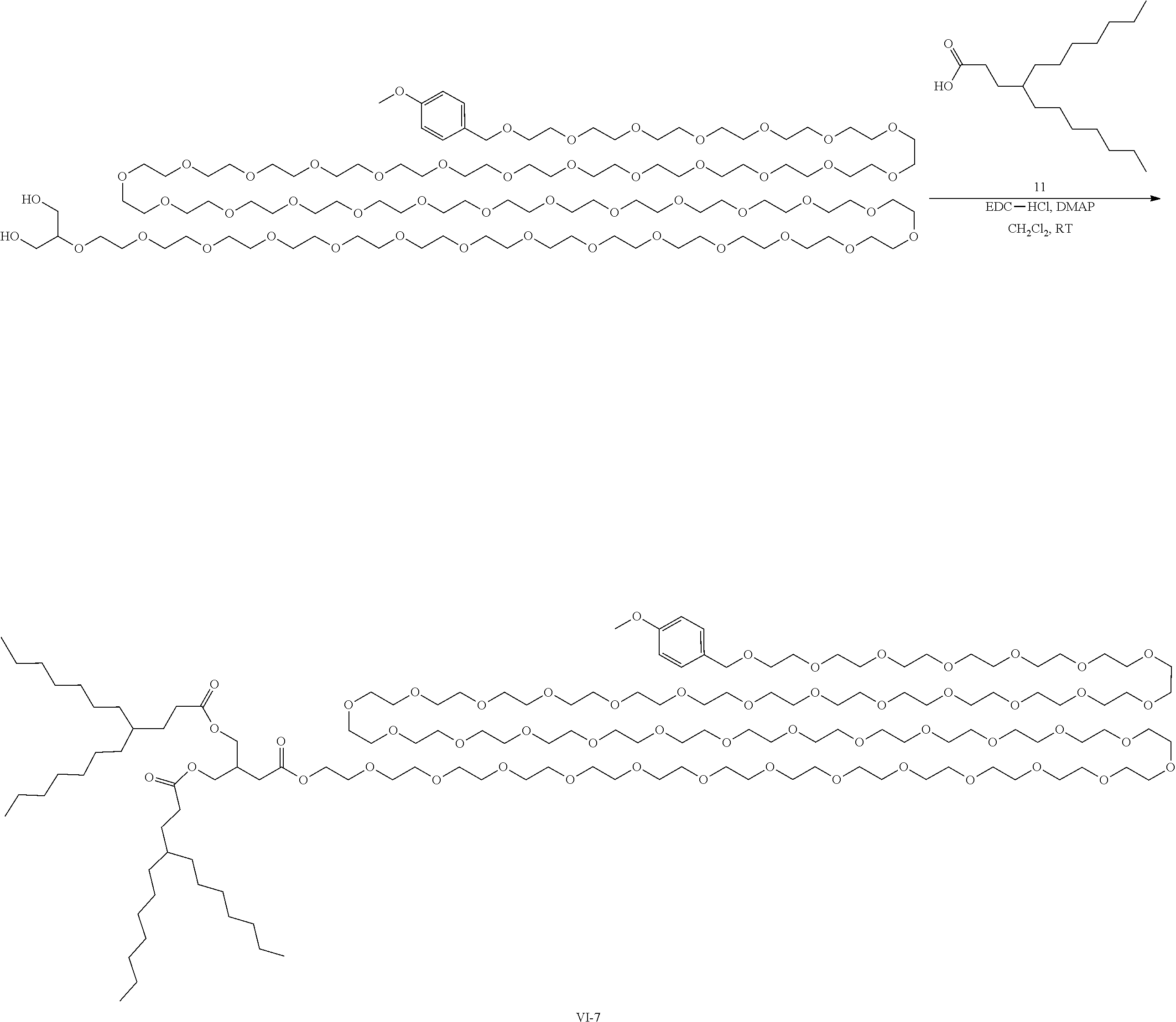

To a solution of VI-6 (6.60 g, 3.01 mmol) in CH₂Cl₂ (330 mL), cooled in an ice-water bath under nitrogen, was added 11 (1.88 g, 6.61 mmol) DMAP (220 mg, 1.80 mmol). The solution was stirred for 10 minutes, then EDC-HCl (1.45 g, 7.56 mmol) was added in portions over 10 minutes. The mixture was warmed to room temperature and was allowed to stir for 16 hours. The solution was diluted with CH₂Cl₂ (330 mL) and washed with 5% aq. NaHCO₃ (330 mL), 5% aq. citric acid (3×110 mL), brine (2×165 mL) and dried (Na₂SO₄). Filtration and concentration in vacuo gave crude VI-7, which was purified by Flash-Prep-HPLC (IntelFlash-1, XB-Phenyl column, gradient CH₃CN/H₂O with 0.05% TFA, from 65% CH₃CN to 95% CH₃CN). Fractions containing VI-7 were pooled, the pH of the solution was adjusted to 7-8 with 5% aq. NH₃ and concentrated to remove the CH₃CN. The resulting solution was diluted with H₂O (150 mL) and was extracted with CH₂Cl₂ (3×150 mL). The combined organic phases were dried over Na₂SO₄, filtered, and concentrated in vacuo to provide VI-7 (5.05 g, 1.85 mmol, 62%) after lyophilization.

¹H-NMR (300 MHz, CDCl₃): δ 7.23 (d, J=8.40 Hz, 2H), 6.87 (d, J=8.40 Hz, 2H), 4.49 (s, 2H), 4.16 (m, 2H), 3.87 (m, 1H), 3.80 (s, 3H), 3.54-3.78 (179H), 3.41 (m, 1H), 2.58 (m, 4H), 1.18-1.38 (50H), 0.90 (m, 12H); HRMS: Calcd. for C₁₃₇H₂₆₄₀₅₁+Na⁺2748.80. Found: 2748.79.

### Example 18. Synthesis of 1,1-Dimethylethyl 4-hydroxy-3-(hydroxymethyl)-2-butenoate (IV-1)

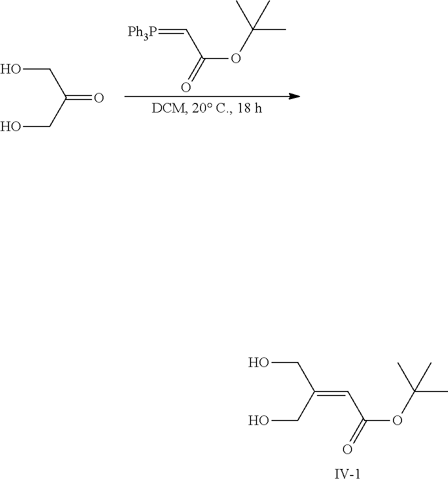

To a solution of dihydroxyacetone (150.0 g, 1.67 mol) in anhydrous dichloromethane (3.0 L), under nitrogen) was added tert-butoxycarbonylmethylene-triphenylphosphorane (627 g, 1.67 mol) in portions over 1 hour. The mixture was stirred for 18 hours at room temperature, then silica gel (750 g, type: ZCX-2, 100-200 mesh) was added to the solution and the solvent was removed in vacuo to afford crude IV-1 impregnated on silica gel. The dry silica gel was placed onto a gravity column of silica gel (3700 g, type: ZCX-2, 100-200 mesh, packed with petroleum ether), and the resulting column was eluted with a gradient of petroleum ether: ethyl acetate (100:0 to 50:50). Compound 1 eluted with petroleum ether: ethyl acetate 50:50 and the fractions of IV-1 were concentrated in vacuo to provide IV-1 (235.0 g) containing Ph₃PO (purity 73.8% by HNMR, 55% yield of IV-1).

¹H NMR (400 MHz, DMSO-d₆, ppm): δ 5.77 (m, 1H), 5.00 (t, J=5.6 Hz, 1H), 4.80 (t, J=5.7 Hz, 1H), 4.49 (dd, J=5.8, 1.5 Hz, 2H), 4.17 (dd, J=5.6, 2.0 Hz, 2H), 1.42 (s, 9H); LCMS (+mode): Calcd. for C₉H₁₆O₄+H⁺: 189.11, Found: 189.10.

### Example 19. Synthesis of tert-Butyl 4-hydroxy-3-(hydroxymethyl)butanoate (IV-2)

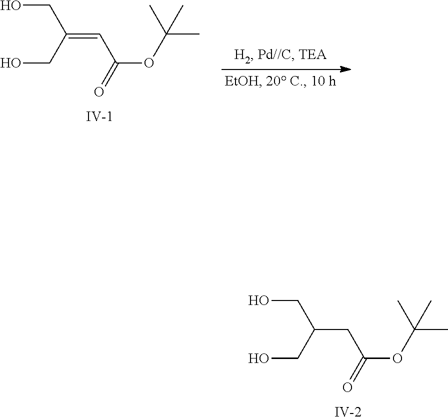

To a solution of IV-1 (100.00 g, 73.8% pure, 0.53 mol) in anhydrous EtOH (1.0 L), in a 2.0 L round bottom flask under nitrogen, was added Et3N (8.10 g, 0.080 mol) followed by 10% Pd/C (20.0 g). The mixture was placed under a hydrogen balloon for 16 hours at room temperature. HPLC analysis indicated that the hydrogenation was not complete and the hydrogen balloon was refilled and the reaction was continued for an additional 16 hours. The mixture was filtered through a pad of Celite®, the filter cake was rinsed with absolute EtOH (200 mL) and the combined filtrates were concentrated in vacuo to afford IV-2(198.0 g) containing P₃PO (purity 67.5% by HNMR, 66% yield of IV-2) as a yellow oil.

¹H NMR (400 MHz, DMSO-D6, ppm): δ 4.47 (t, J=5.10 Hz, 2H), 3.44-3.29 (5H), 2.17 (d, J=7.00 Hz, 2H), 1.93 (m, 1H), 1.39 (s, 9H).

### Example 20. Synthesis of 2-(2-(tert-Butoxy)-2-oxoethyl)propane-1,3-diyl distearate (IV-3)

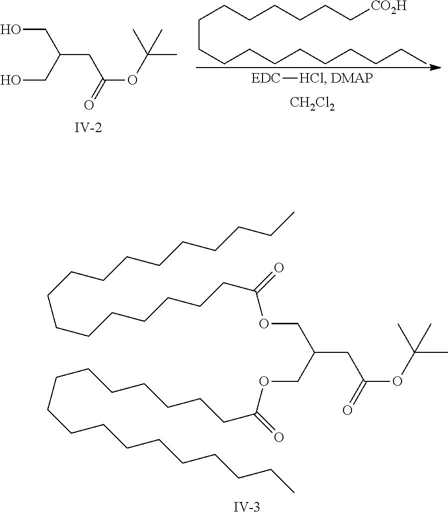

To a solution of IV-2(2.00 g, 10.51 mmol) in CH₂Cl₂ (200 mL), cooled in an ice-water bath under nitrogen, was added in order stearic acid (6.60 g, 26.6 mmol) and DMAP (0.90 g, 7.36 mmol). The mixture was stirred for 10 minutes, then EDC-HCl (5.10 g, 26.60 mmol) was added in portions over 15 minutes. After the addition was complete, the mixture was warmed to room temperate and was stirred for 12 hours. The mixture was cast into water (100 mL), the organic phase was separated, the aqueous phase was extracted with CH₂Cl₂ (2×200 mL), and the combined organic phases were dried over Na₂SO₄. Filtration and concentration in vacuo gave crude IV-3 which was dissolved in CH₂Cl₂ (100 mL), then silica gel (20 g, type: ZCX-2, 100-200 mesh) was added to the solution and the solvent was removed in vacuo to afford crude IV-3 impregnated on silica gel. The dry silica gel was placed onto a gravity column of silica gel (175 g, type: ZCX-2, 100-200 mesh, packed with n-heptane), and the resulting column was eluted with a gradient of n-heptane: ethyl acetate (100:0 to 98:2). Fractions containing IV-3 were pooled and concentrated in vacuo to provide IV-3 (4.50 g, 6.22 mmol, 59%) as a white solid.

¹H NMR (400 MHz, CDCl₃, ppm): δ 4.12 (m, 4H), 2.52 (m, 1H), 2.32 (m, 6H), 1.63 (m, 4H), 1.47 (s, 9H), 1.20-1.44 (56H), 0.90 (t, J=6.40 Hz, 6H); LCMS: Calcd. for C₄₅H₈₆O₆+Na⁺745.63. Found: 745.75.

### Example 21. Synthesis of 4-(Stearoyloxy)-3-((stearoyloxy)methyl)butanoic acid (15)

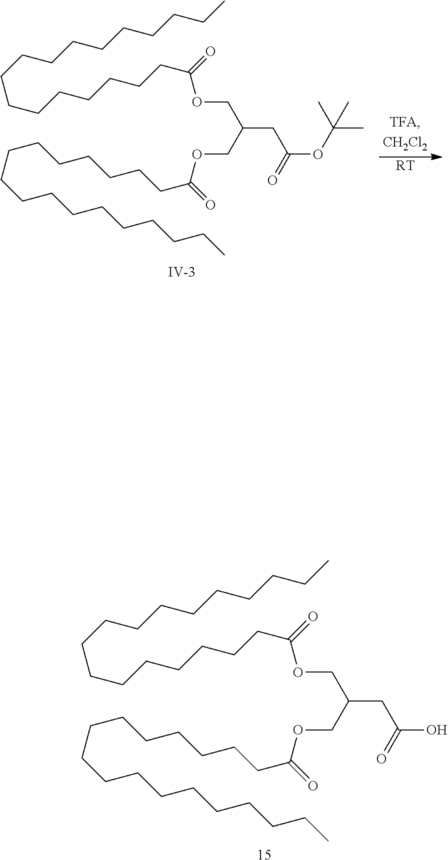

To a solution of IV-3 (4.00 g, 5.50 mmol) in CH₂Cl₂ (40 mL), cooled in an ice-water bath under nitrogen, was added TFA (8.00 mL, 11.91 g, 0.104 mol). The mixture was stirred for 5 minutes, then the solution was warmed to room temperature and was stirred for 2 hours. The solution was concentrated in vacuo to give a semi-solid which was triturated with H₂O (20 mL). The resulting solid was isolated by filtration, the filter cake was rinsed with water (3×8 mL), the solid was triturated with n-heptane (40 mL) and the solid was isolated by filtration. The filter cake was rinsed with n-heptane (3×8 mL), the solid was triturated with CH₃CN (40 mL), and the solid was isolated by filtration. The solid was dried in vacuo to provide 15 (2.50 g, 3.75 mmol, 68%) as a white solid.

¹H NMR (400 MHz, CDCl₃, ppm): δ 4.15 (m, 4H), 2.59 (m, 1H), 2.49 (m, 2H), 2.33 (m, 4H), 1.63 (m, 4H), 1.20-1.50 (56H), 0.90 (t, J=6.80 Hz, 6H); LCMS: Calcd. for C₄₁H₇₈O₆+Na⁺689.57. Found: 689.70.

### Example 22. Synthesis of Compound B-46

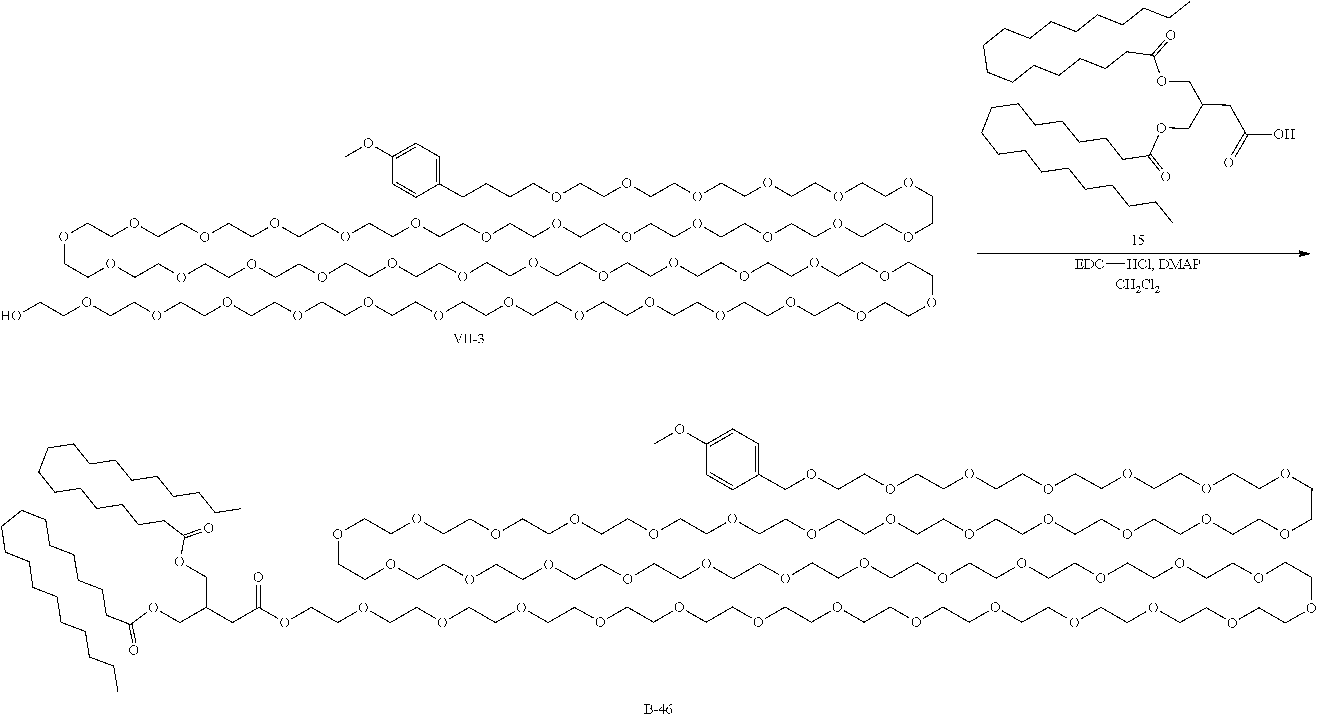

To a solution of 15 (2.20 g, 3.30 mmol) and DMAP (403 mg, 3.30 mmol) in CH₂Cl₂ (300 mL), cooled in an ice-water bath under nitrogen, was VII-3 (5.83 g, 2.75 mmol). The solution was stirred for 10 minutes, then EDC-HCl (1.27 g, 6.62 mmol) was added over a period of 5 minutes. The mixture was stirred for 30 minutes, then was warmed to room temperature, and stirring was continued for 16 hours. The mixture was cast into CH₂Cl₂ (300 mL) and the resulting solution was washed with 5% aq. NaHCO₃ (300 mL), 5% aq. citric acid (3×150 mL), brine (2×175 mL). The organic phase was separated and dried over Na₂SO₄. Filtration and concentration in vacuo gave crude B-46 which was purified by Flash-Prep-HPLC (IntelFlash-1, XB-Phenyl column, gradient CH₃CN/H₂O with 0.05% TFA, from 65% CH₃CN to 95% CH₃CN). Fractions containing B-46 were pooled, the pH of the solution was adjusted to 7-8 with 5% aq. NH₃ and concentrated to remove the CH₃CN. The resulting solution was diluted with H₂O (150 mL) and was extracted with CH₂Cl₂ (3×150 mL). The combined organic phases were dried over Na₂SO₄, filtered, and concentrated in vacuo to provide B-46 (5.10 g, 1.84 mmol, 67%) as a white solid after lyophilization.

¹H-NMR (300 MHz, CDCl₃): δ 7.27 (d, J=8.40 Hz, 2H), 6.87 (d, J=8.40 Hz, 2H), 4.50 (s, 2H), 4.25 (m, 2H), 4.11 (m, 4H), 3.88 (m, 1H), 3.80 (s, 3H), 3.60-3.77 (176H), 3.41 (m, 1H), 2.56 (m, 1H), 2.44 (m, 2H), 2.27 (m, 4H), 1.60 (m, 4H), 1.18-1.38 (56H), 0.88 (m, 6H); HRMS: Calcd. for C₁₃₉H₂₆₆O₅₂+Na⁺2790.81. Found 2790.81.

### Example 23. Synthesis of 2-(2-(tert-Butoxy)-2-oxoethyl)propane-1,3-diyl bis(4-heptylundecanoate) (IV-4)

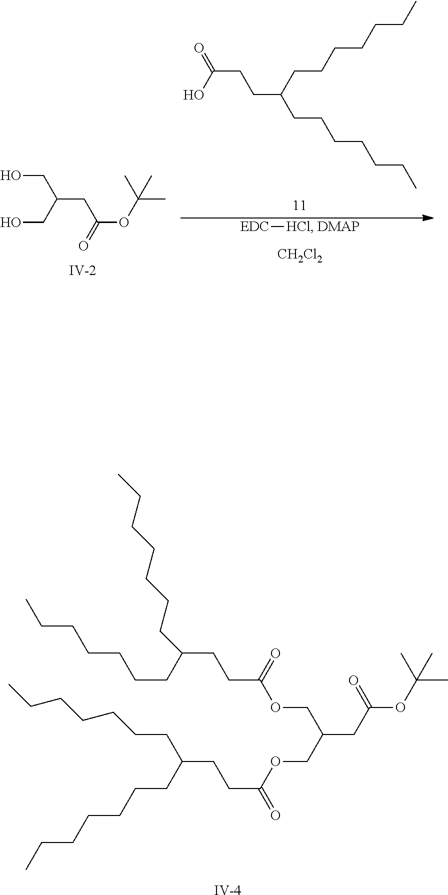

To a solution of 11 (6.25 g, 22.0 mmol) and IV-2 (1.90 g, 10.00 mmol) in CH₂Cl₂ (95 mL), cooled in an ice-water bath under nitrogen, was added DMAP (610 mg, 5.0 mmol). The solution was stirred for 10 minutes, then EDC-HCl (4.80 g, 25.0 mmol) was added in portions over 15 minutes. The mixture was stirred for 30 minutes, then was warmed to room temperature and was allowed to stir for an additional 16 hours. The mixture was cast into water, the organic phase was separated, the aqueous phase was extracted with CH₂Cl₂ (2×100 mL) and the combined organic phases were dried over Na₂SO₄. Filtration and concentration in vacuo provided crude IV-4 which was dissolved in CH₂Cl₂ (50 mL), then silica gel (20 g, type: ZCX-2, 100-200 mesh) was added to the solution and the solvent was removed in vacuo to afford crude IV-4 impregnated on silica gel. The dry silica gel was placed onto a gravity column of silica gel (180 g, type: ZCX-2, 100-200 mesh, packed with n-heptane), and the resulting column was eluted with a gradient of n-heptane: ethyl acetate (100:0 to 98:2). Fractions containing IV-4 were pooled and concentrated in vacuo to provide IV-4 (6.50 g, 8.98 mmol, 90%) as a colorless oil.

¹H-NMR (300 MHz, CDCl₃): δ 4.10 (m, 4H), 2.55 (m, 1H), 2.23-2.36 (6H), 1.59 (m, 4H), 1.45 (s, 9H), 1.15-1.40 (50H), 0.88 (m, 12H); LCMS: Calcd. for C₄₅H₈₆O₆+Na⁺745.63. Found 745.90.

### Example 24. Synthesis of 4-((4-Heptylundecanoyl)oxy)-3-(((4-heptylundecanoyl)oxy)methyl)butanoic acid (17)

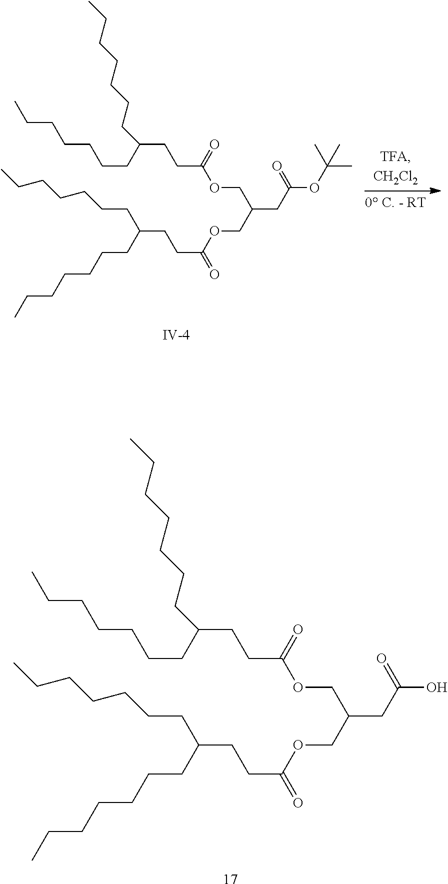

A solution of IV-4 (6.00 g, 8.30 mmol) in CH₂Cl₂(30 mL), cooled in an ice-water bath under nitrogen, was treated with TFA (1.40 mL, 2.05 g, 18.0 mmol). The mixture was stirred for 30 minutes, then was allowed to warm to room temperature and stir for an additional 2 hours. The mixture was concentrated in vacuo and the residue was dissolved in ethyl acetate (60 mL). The resulting solution was washed with 2% aq. NaHCO₃ (30 mL), 5% aq. citric acid (30 mL), brine (2×15 mL), and dried (Na₂SO₄). Filtration and concentration in vacuo led to 17 (5.10 g, 7.62 mmol, 92%), as a clear, pale-yellow oil.

¹H-NMR (300 MHz, CDCl₃): δ 4.12 (m, 4H), 2.55 (m, 1H), 2.55 (m, 2H), 2.27 (m, 4H), 1.60 (m, 4H), 1.35-1.40 (50H), 0.88 (m, 12H).

### Example 25. Synthesis of Compound B-47

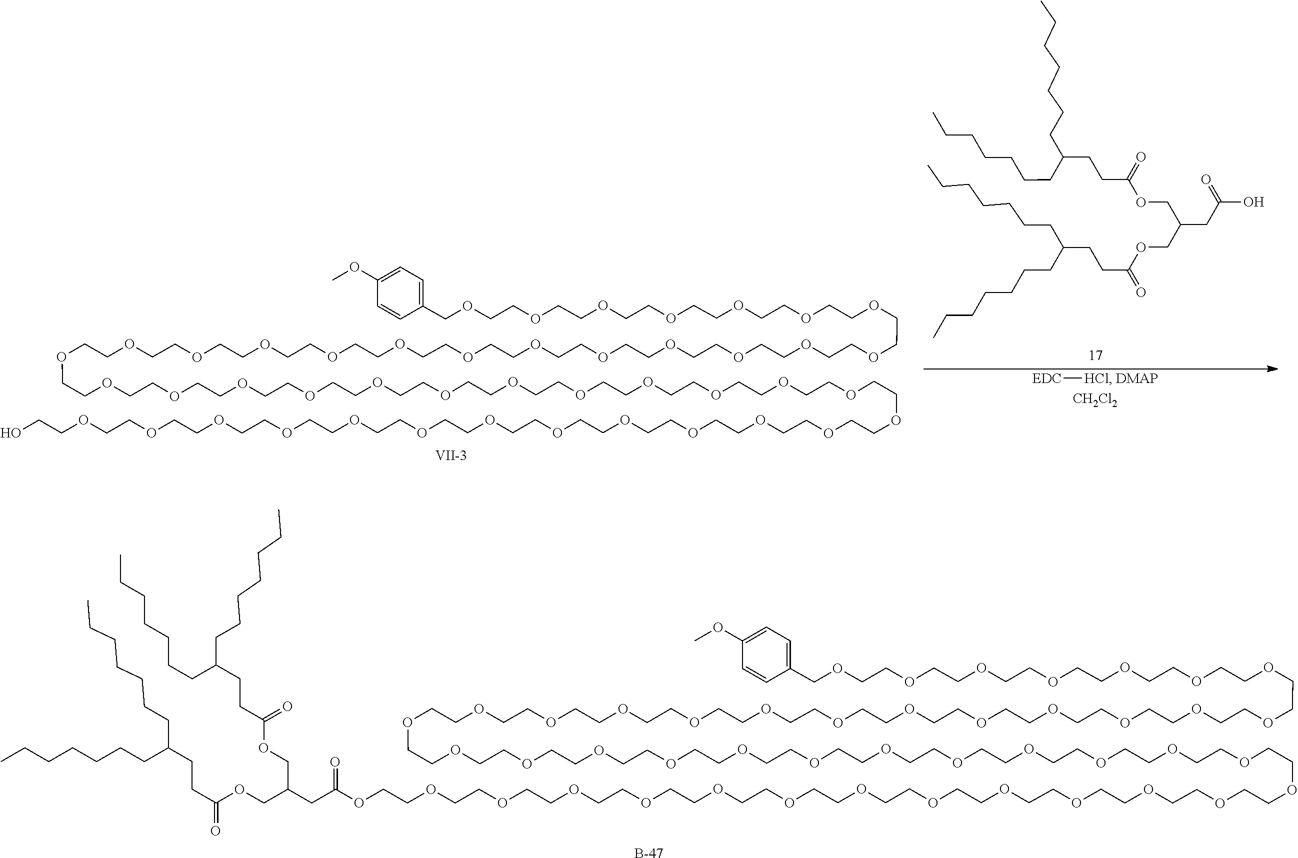

To a solution of VII-3 (7.00 g, 3.30 mmol) in CH₂Cl₂ (350 mL), cooled in an ice-water bath under nitrogen, was added 17 (2.42 g, 3.63 mmol) and DMAP (0.793 g, 6.50 mmol). The mixture was stirred for 10 minutes, then EDC-HCl (0.791 g, 4.13 mmol) was added in one portion. After the addition was complete, the mixture was stirred for 10 minutes, then was warmed to room temperature and stirred for 16 hours. The solution was cast into CH₂Cl₂ (350 mL) and 5% aq. NaHCO₃ (350 mL). The organic phase was separated, washed with 5% aq. citric acid (3×140 mL), brine (2×200 mL) and dried over Na₂SO₄. Filtration and concentration in vacuo gave crude B-47 which was purified by Flash-Prep-HPLC (IntelFlash-1, XB-Phenyl column, gradient CH₃CN/H₂O with 0.05% TFA, from 65% CH₃CN to 95% CH₃CN). Fractions containing B-47 were pooled, the pH of the solution was adjusted to 7-8 with 5% aq. NH₃ and concentrated to remove the CH₃CN. The resulting solution was diluted with H₂O (150 mL) and was extracted with CH₂Cl₂ (3×150 mL). The combined organic phases were dried over Na₂SO₄, filtered, and concentrated in vacuo to provide B-47 (5.04 g, 1.82 mmol, 60%) as a white solid after lyophilization.

¹H-NMR (300 MHz, CDCl₃): δ 7.26 (d, J=8.40 Hz, 2H), 6.87 (d, J=8.40 Hz, 2H), 4.50 (s, 2H), 4.25 (m, 2H), 4.10 (m, 4H), 3.90 (m, 1H), 3.80 (s, 3H), 3.60-3.77 (176H), 3.43 (m, 1H), 2.56 (m, 1H), 2.44 (m, 2H), 2.28 (m, 4H), 1.60 (m, 4H), 1.18-1.38 (50H), 0.88 (m, 12H); HRMS: Calcd. for C₁₃₉H₂₆₆O₅₂+Na⁺2790.81. Found 2790.80.

### Example 26. Synthesis of PEG-Alcohol B-24

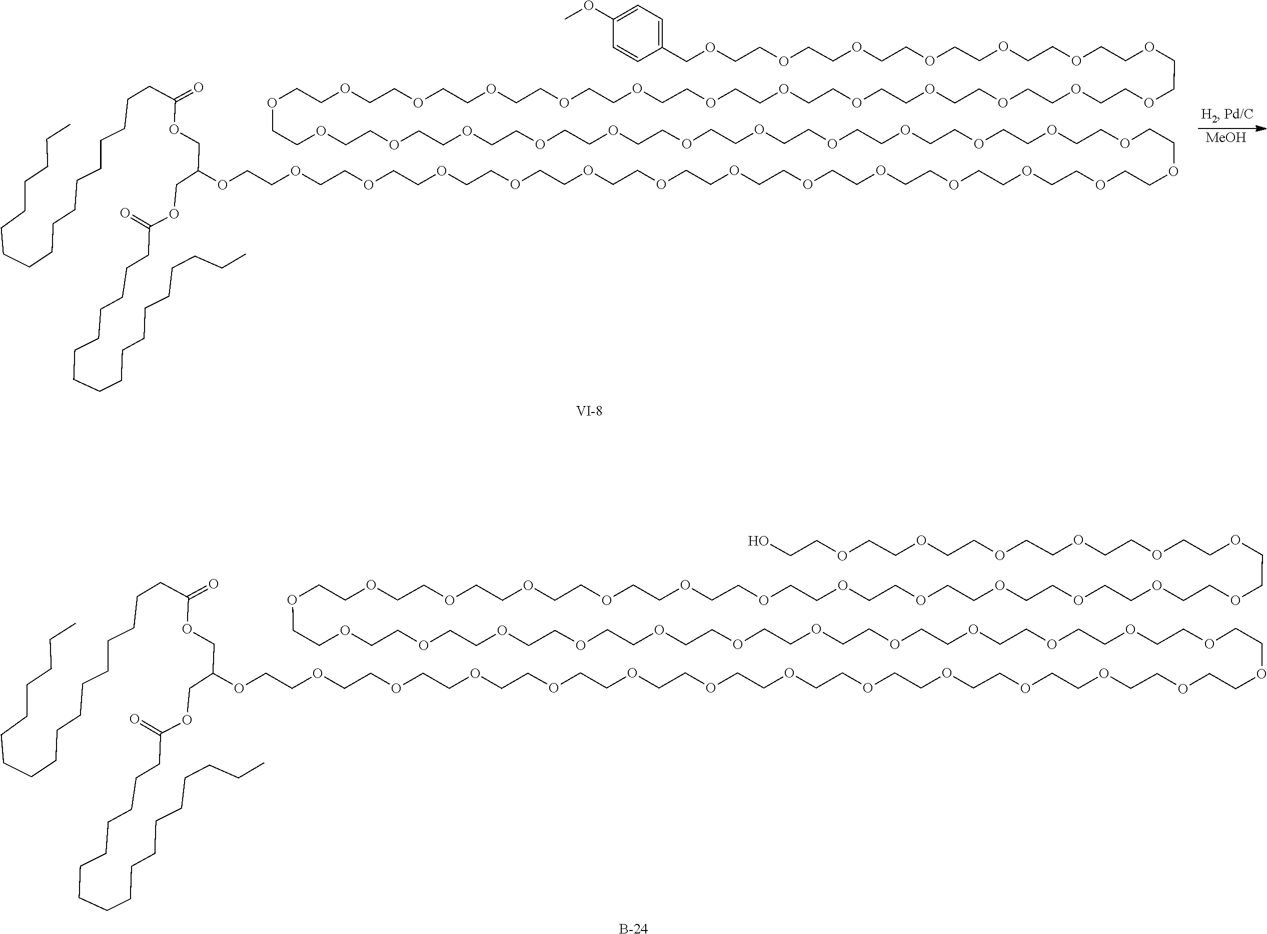

To a solution of VI-8 (1.50 g, 0.55 mmol) in MeOH (38 mL), under nitrogen at room temperature, was added 10% Pd/C (150 mg). The flask was evacuated and flushed with nitrogen (3×), then the mixture was placed under a balloon of nitrogen. The mixture was stirred for 2 hours under the hydrogen balloon, then was carefully filtered through a pad of Celite, the filter cake was rinsed with MeOH (25 mL), and the combined filtrates were concentrated in vacuo to afford alcohol B-24 (1.20 g, 0.46 mmol, 84%) as a white solid.

¹H-NMR (300 MHz, CDCl₃): δ 4.17 (m, 4H), 3.55-3.87 (182H), 2.55 (brs, 1H), 2.31 (t, J=7.20 Hz, 4H), 1.60 (m, 4H), 1.15-1.37 (56H), 0.87 (m, 6H); HRMS: Calcd. for C₁₂₉H₂₅₆O₅₀+Na⁺2628.74, Found 2628.94.

### Example 27. Synthesis of PEG-Mesylate 19

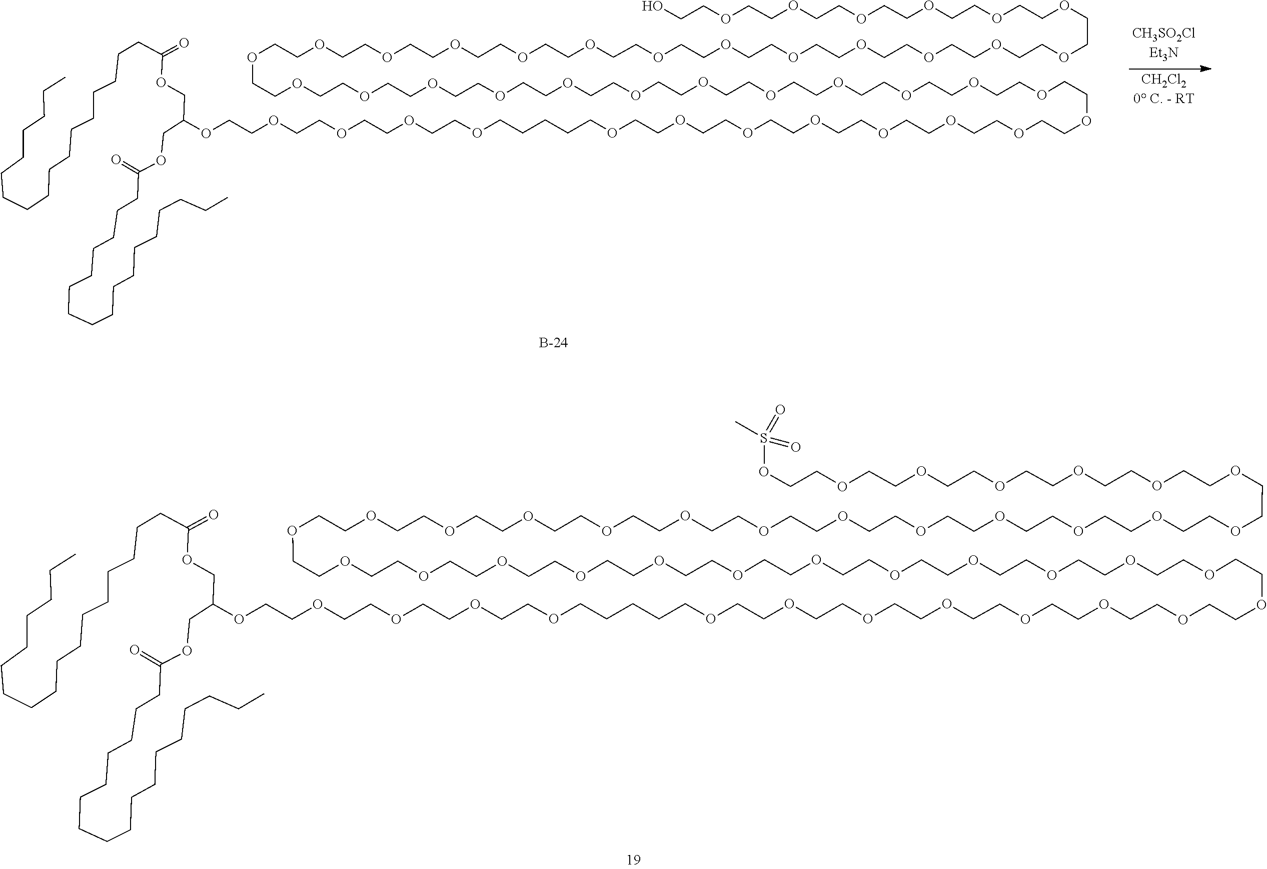

To a solution of B-24 (1.20 g, 0.46 mmol) in CH₂Cl₂ (26 mL), cooled in an ice-water bath under nitrogen, was added Et3N (0.23 g, 2.30 mmol) followed by the dropwise addition of a solution of CH₃SO₂Cl (MsCl, 0.16 g, 1.38 mmol) in CH₂Cl₂ (5 mL) over a period of 5 minutes. The mixture was stirred for 1 hour then was warmed to room temperature and was stirred for an additional 14 hours. The mixture was cast into CH₂Cl₂ (20 mL) and H₂O (10 mL). The organic phase was separated, the aq. phase was extracted with CH₂Cl₂ (20 mL), and the combined organic phases were washed with 1 N aq. HCl (50 mL), H₂O (2×25 mL) and dried over Na₂SO₄. Filtration and concentration in vacuo gave 19 (1.20 g, 0.44 mmol, 96%) as a pale-yellow solid.

HRMS: Calcd. for C₁₃₀H₂₅₈O₅₂+H⁺2684.73, Found 2628.73.

### Example 28. Synthesis of PEG-Azide 20

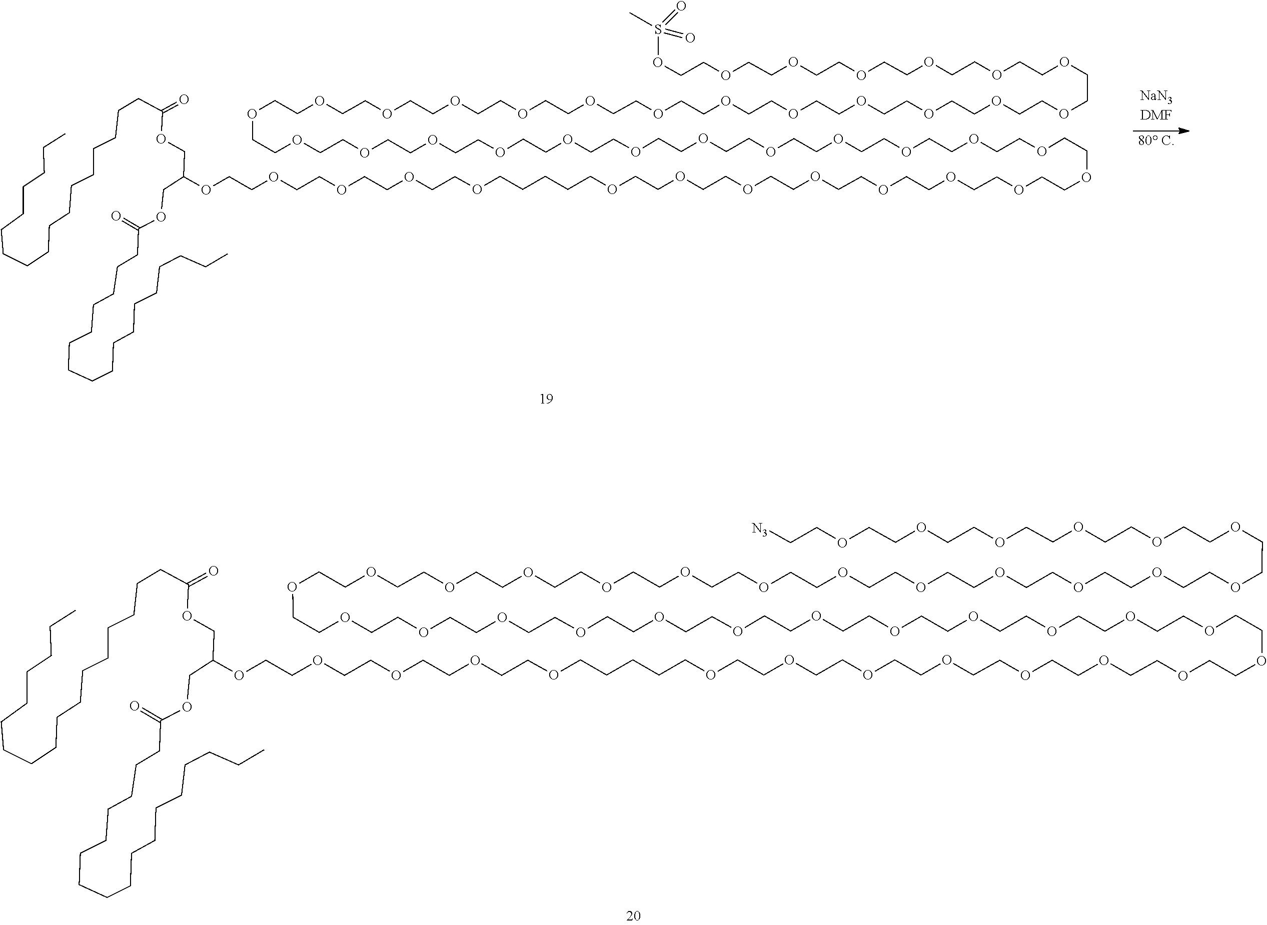 [SMILES: CCCCCCCCCCCCCCCCCC(=O)OCC(COC(=O)CCCCCCCCCCCCCCCCC)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOS(C)(=O)=O ; "80° C." ; CCCCCCCCCCCCCCCCCC(=O)OCC(COC(=O)CCCCCCCCCCCCCCCCC)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCN=[N+]=[N-]]

To a solution of 19 (1.50 g, 0.56 mmol) in CH₃CN (30 mL), under nitrogen, was added NaN₃ (0.11 g, 1.68 mmol) in one portion. The solution was then warmed to 80° C. and was stirred for 24 hours. The mixture was cooled to room temperature and concentrated in vacuo to afford crude 20. Crude 20 was purified by chromatography on a column of silica gel (150 g, type ZCX-2, 100-200 mesh) packed with CH₂Cl₂ and eluted with a gradient of CH₂Cl₂/MeOH from 95:5 to 80:20. Fractions containing 20 were pooled and concentrated in vacuo to provide 20 (1.20 g, 0.456 mmol, 81%) as a pale-yellow solid.

¹H-NMR (300 MHz, CDCl₃): δ 4.17 (m, 4H), 3.65-3.87 (184H), 3.30-3.45 (4H), 2.31 (t, J=7.20 Hz, 4H), 1.60 (m, 4H), 1.15-1.37 (56H), 0.87 (m, 6H); HRMS: Calcd. for C₁₂₉H₂₅₃N₃O₄₉+Na⁺2653.75, Found 2653.75.

### Example 29. Synthesis of PEG-Amine 21

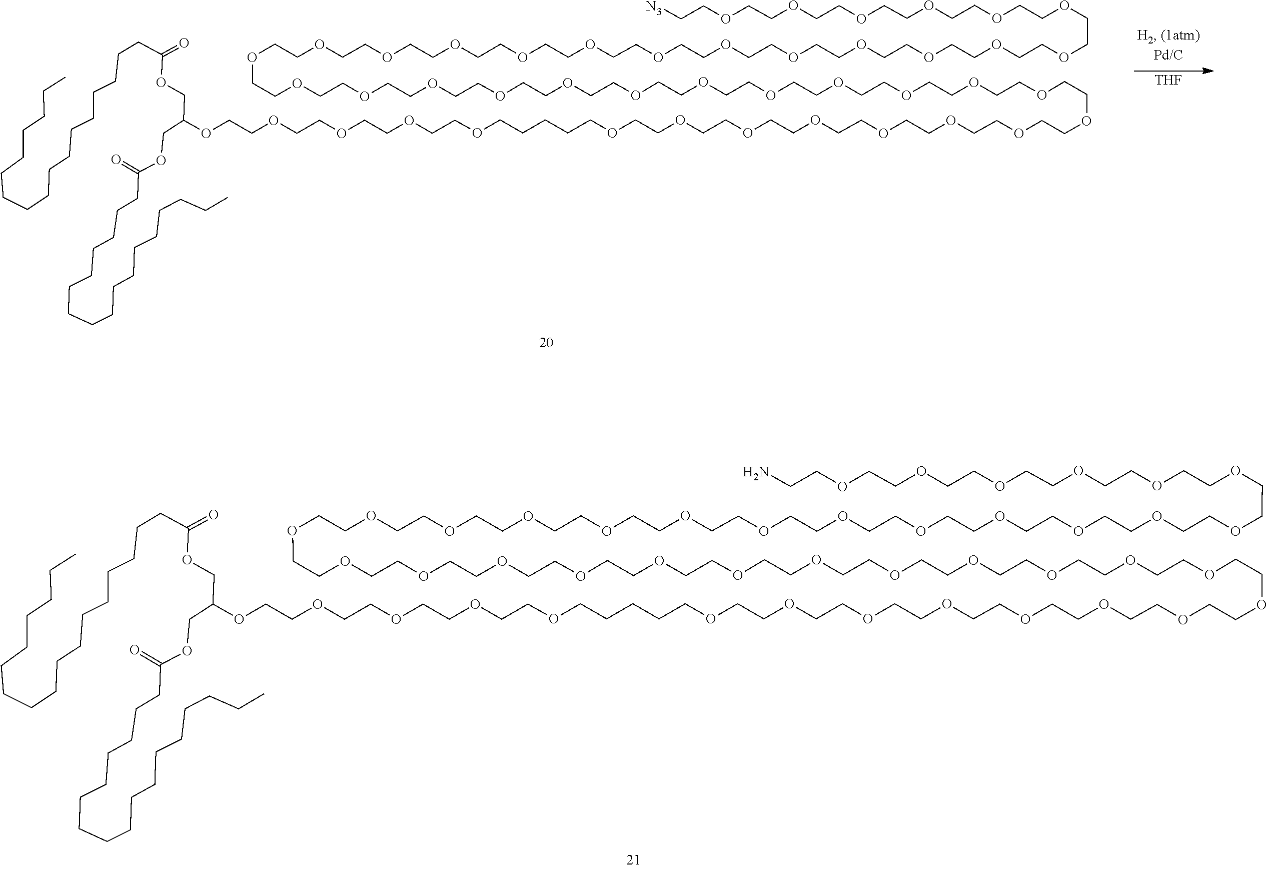

To a solution of azide 20 (1.10 g, 0.418 mmol), in THE (110 mL) under nitrogen, was added 10% Pd/C. The mixture was evacuated and refilled with nitrogen (3×), then the mixture was placed under a balloon of hydrogen. The mixture was stirred for 16 hours, then the solids were removed by filtration through a pad of Celite, the filter cake was rinsed with THE (25 mL) and the combined filtrates were concentrated in vacuo to yield 21 (0.893 g, 0.343 mmol, 82%) as a sticky, pale-yellow solid.

¹H-NMR (300 MHz, CDCl₃): δ 4.10 (m, 4H), 3.62-3.71 (10H), 3.50-3.60 (174H), 2.19-2.34 (6H), 1.76-1.85 (6H), 1.34-1.42 (6H), 1.15-1.26 (50H), 0.87 (m, 6H); HRMS: Calcd. for C₁₂₉H₂₅₇NO₄₉+H⁺2605.77, Found 2605.78.

### Example 30. Synthesis of PEG-Bromomaleimide Amide 22

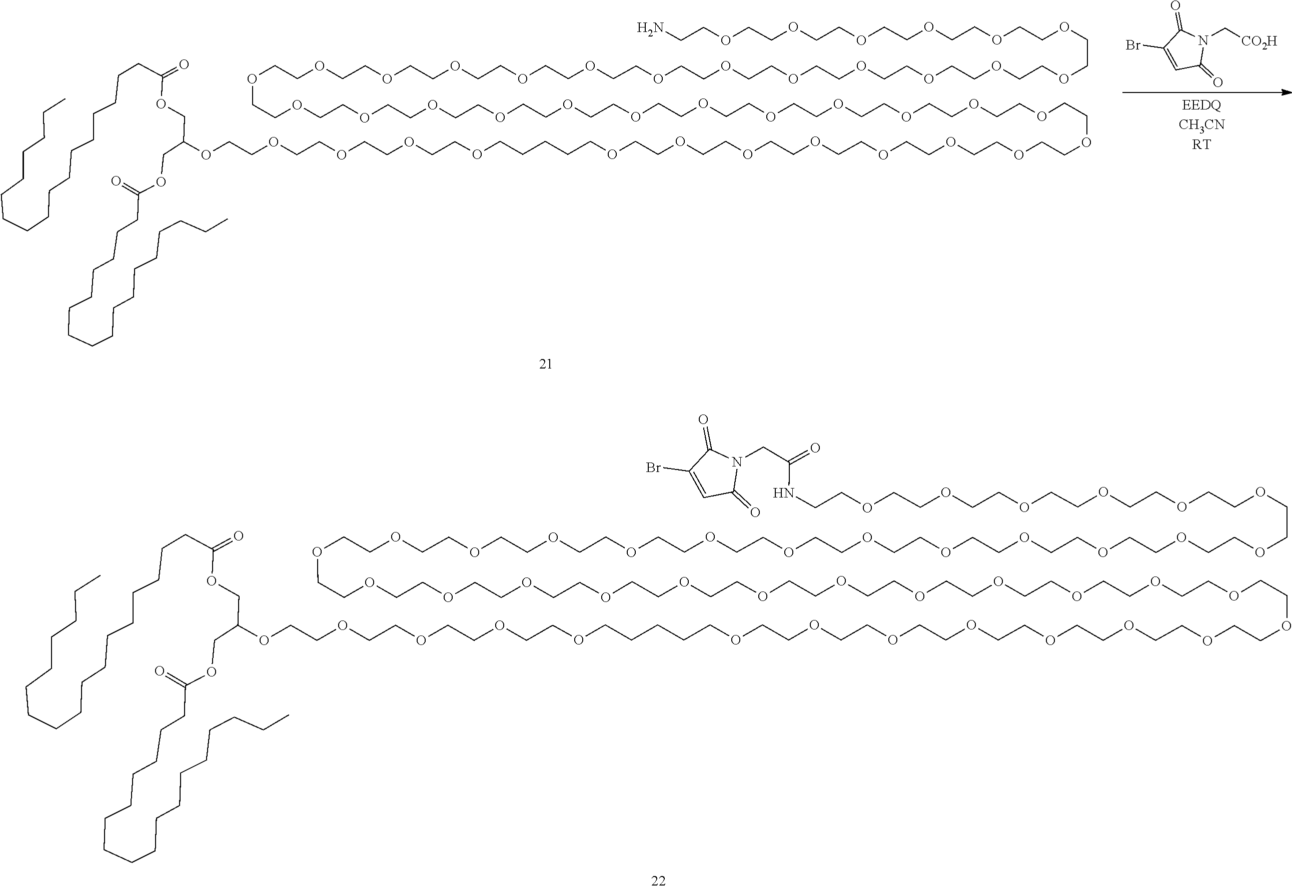 [SMILES: CCCCCCCCCCCCCCCCCC(=O)OCC(COC(=O)CCCCCCCCCCCCCCCCC)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCN ; O=C(O)CN1C(=O)C=C(Br)C1=O ; CCCCCCCCCCCCCCCCCC(=O)OCC(COC(=O)CCCCCCCCCCCCCCCCC)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CN1C(=O)C=C(Br)C1=O]

A solution of 2-(3-bromo-2,5-dioxo-2,5-dihydro-1H-pyrrol-1-yl)acetic acid (0.100 g, 0.422 mmol) and ethyl 2-ethoxy-2H-quinoline-1-carboxylate (EEDQ, 0.123 g, 0.498 mmol) in CH₃CN (50 mL) was stirred for 1 hour at room temperature under nitrogen, then 21 (1.00 g, 0.384 mmol) was added in one portion. The resulting solution was stirred for 12 hours at room temperature, then the solvent was removed in vacuo to give crude 22 as a sticky yellow, semi-solid. Crude 22 was purified by Flash-Prep-HPLC (Xbridge BEH Phenyl column, gradient i-PrOH—CH₃CN-MeOH (30:20:50)/MeOH—H₂O-0.1% formic acid 80:20 to 85:15). Fractions containing 22 were pooled and the solvent was removed by lyophilization to provide 22 (160 mg, 0.057 mmol, 15%) as an off-white solid.

¹H-NMR (400 MHz, CDCl₃): δ 6.94 (s, 1H), 6.90 (brs, 1H), 4.36 (s, 2H), 4.20 (m, 4H), 3.40-3.90 (179H), 2.33 (t, J=7.20H, 4H), 1.62 (m, 4H), 1.06-1.48 (54H), 0.90 (m, 6H); HRMS: Calcd. for C₁₃₅H₂₅₉BrN₂O₅₂+Na⁺2843.68/2845.68, Found 2843.70/2845.69.

### Example 31. Synthesis of PEG-Alcohol (B-23)

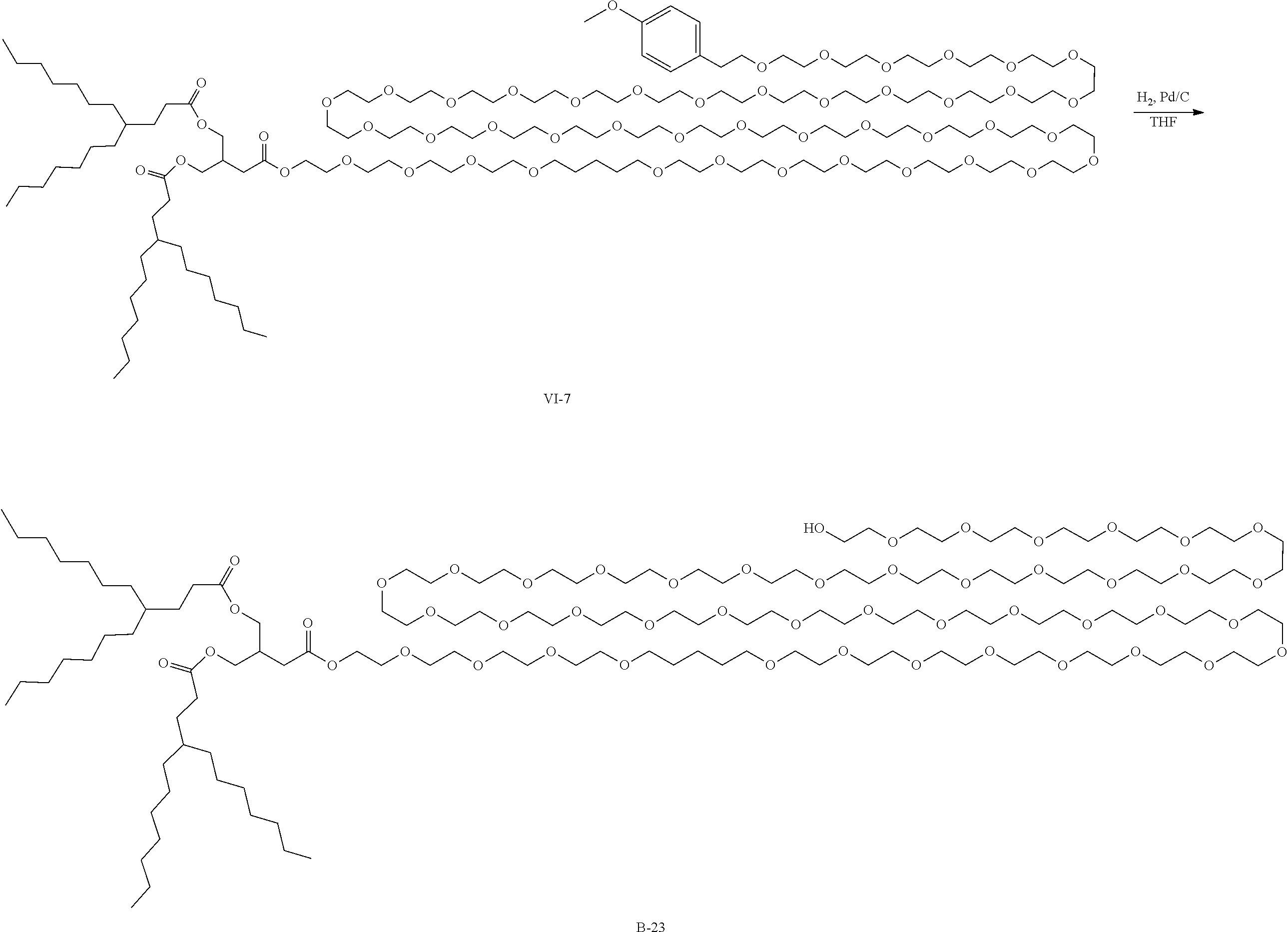

To a solution of VI-7 (3.00 g, 1.10 mmol) in THF (75 mL), under nitrogen, was added 10% Pd/C (600 mg). The system was evacuated and refilled with nitrogen (3×), then a balloon of hydrogen was attached and the mixture was stirred for 6 hours. The solids were removed by filtration through a pad of Celite, the filter cake was rinsed with THF (30 mL), and the combined filtrates were concentrated in vacuo to provide B-23 (2.48 g, 0.951 mmol, 86%) as an off-white solid.

¹H-NMR (300 MHz, CDCl₃): δ 4.18 (m, 4H), 3.60-3.87 (180H), 2.30 (t, J=7.50 Hz, 4H), 1.59 (m, 4H), 1.15-1.38 (50H), 0.90 (m, 12H); HRMS: Calcd. for C₁₂₉H₂₅₆O₅₀+H⁺2606.76. Found: 2606.76.

### Example 32. Synthesis of PEG-Mesylate 24

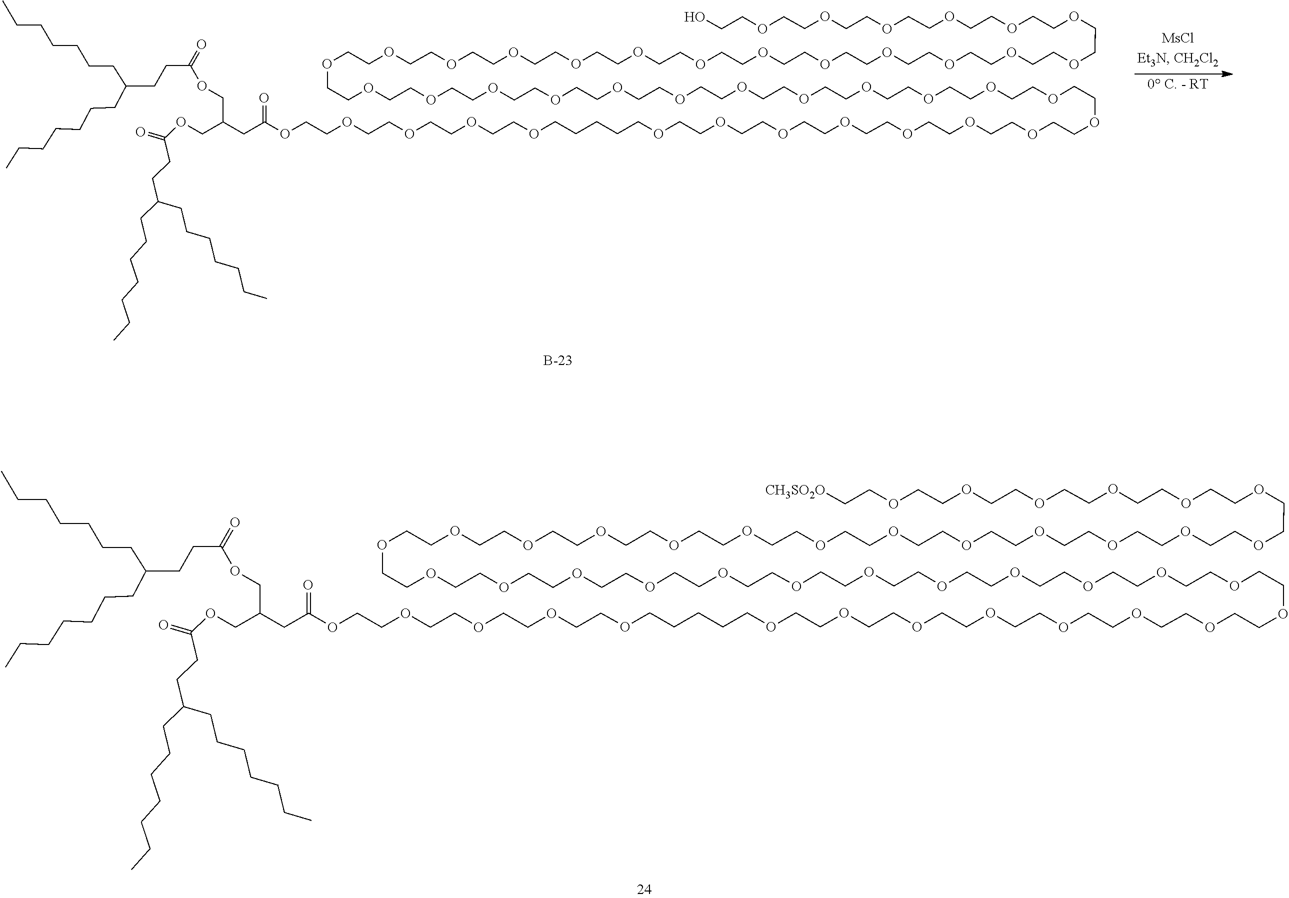

To a solution of B-23 (0.67 g, 0.256 mmol) in CH₂Cl₂ (14 mL), in an ice-water bath under nitrogen, was added Et3N (78 mg, 0.77 mmol). The mixture was allowed to stir for 10 minutes, then MsCl (26.3 mg, 0.230 mmol) was added in one portion. After the addition was complete, the solution was allowed to warm to room temperature and was stirred for 14 hours. The solution was cast into CH₂Cl₂ (20 mL) and H₂O (10 mL), The organic phase was separated, the aq. layer was extracted with CH₂Cl₂ (20 mL) and the combined organic layers were washed with 1 N aq. HCl (20 mL), water (2×10 mL), and dried (Na₂SO₄). Filtration and concentration in vacuo gave crude 24 (0.680 g, 0.253 mmol, 99%) as a viscous, light-yellow oil.

HRMS: Calcd. for C₁₃₀H₂₅₈O₅₂+Na⁺2706.72. Found: 2706.72.

### Example 33. Synthesis of PEG-Azide 25

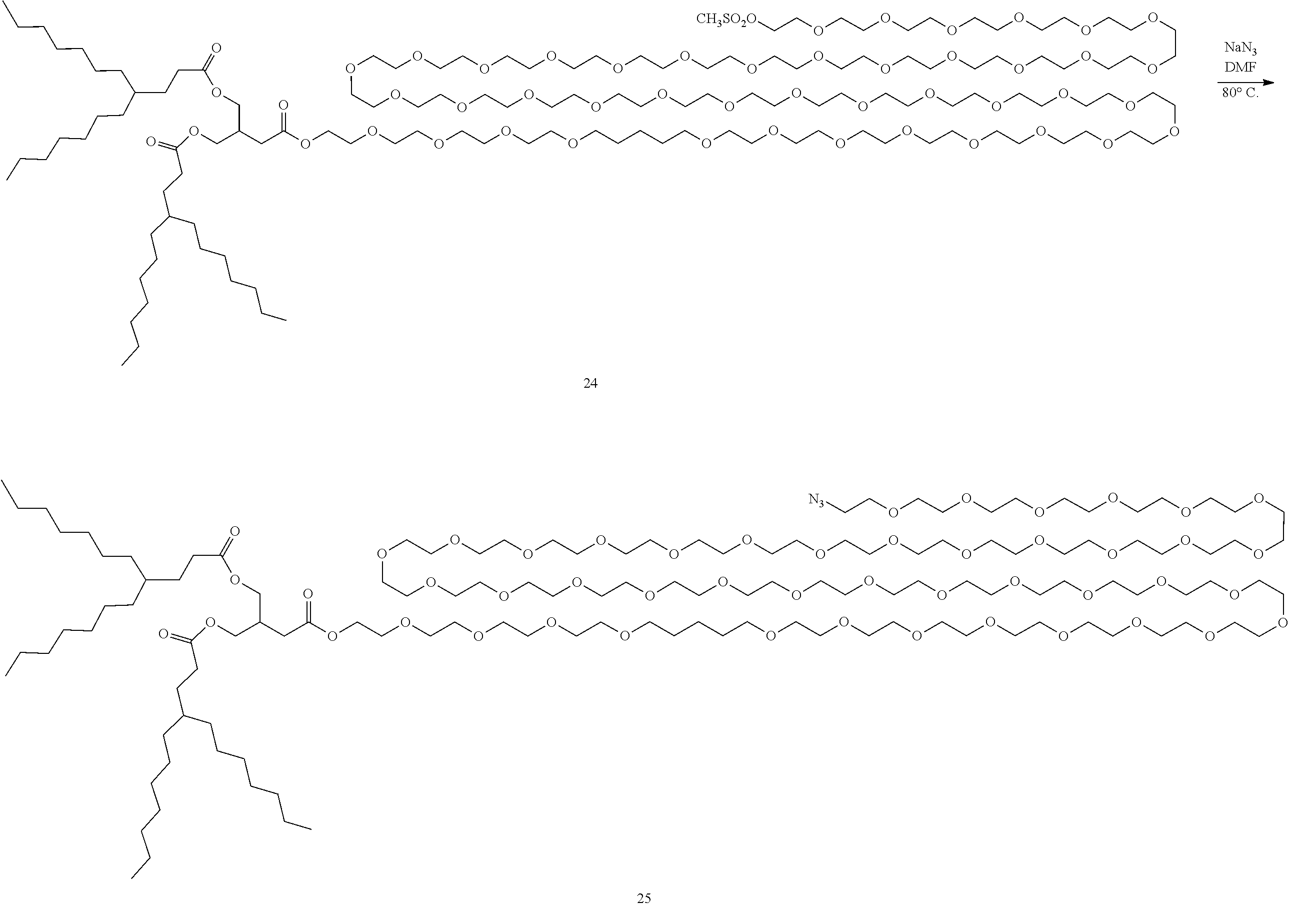

To a solution of mesylate 24 (700 mg, 0.260 mmol) in DMF (20 mL), was added sodium azide (32.5 mg, 0.50 mmol), at room temperature under nitrogen. After the addition, the mixture was warmed to 80° C. and stirred for 14 hours. The mixture was cooled to room temperature and was purified by Prep-reverse phase HPLC (C₁₈ column, CH₃CN-MeOH (3:1)/H₂O w/0.1% TFA, gradient from 30% to CH₃CN-MeOH. Fractions containing 25 were pooled and the solvent was removed by lyophilization to give 25 (550 mg, 0.209 mmol, 80%) as a waxy, off-white solid.

HRMS: Calcd. for C₁₂₉H₂₅₅N₃O₄₉+Na⁺2653.75. Found: 2653.74.

### Example 34. Synthesis of PEG-Amine 26

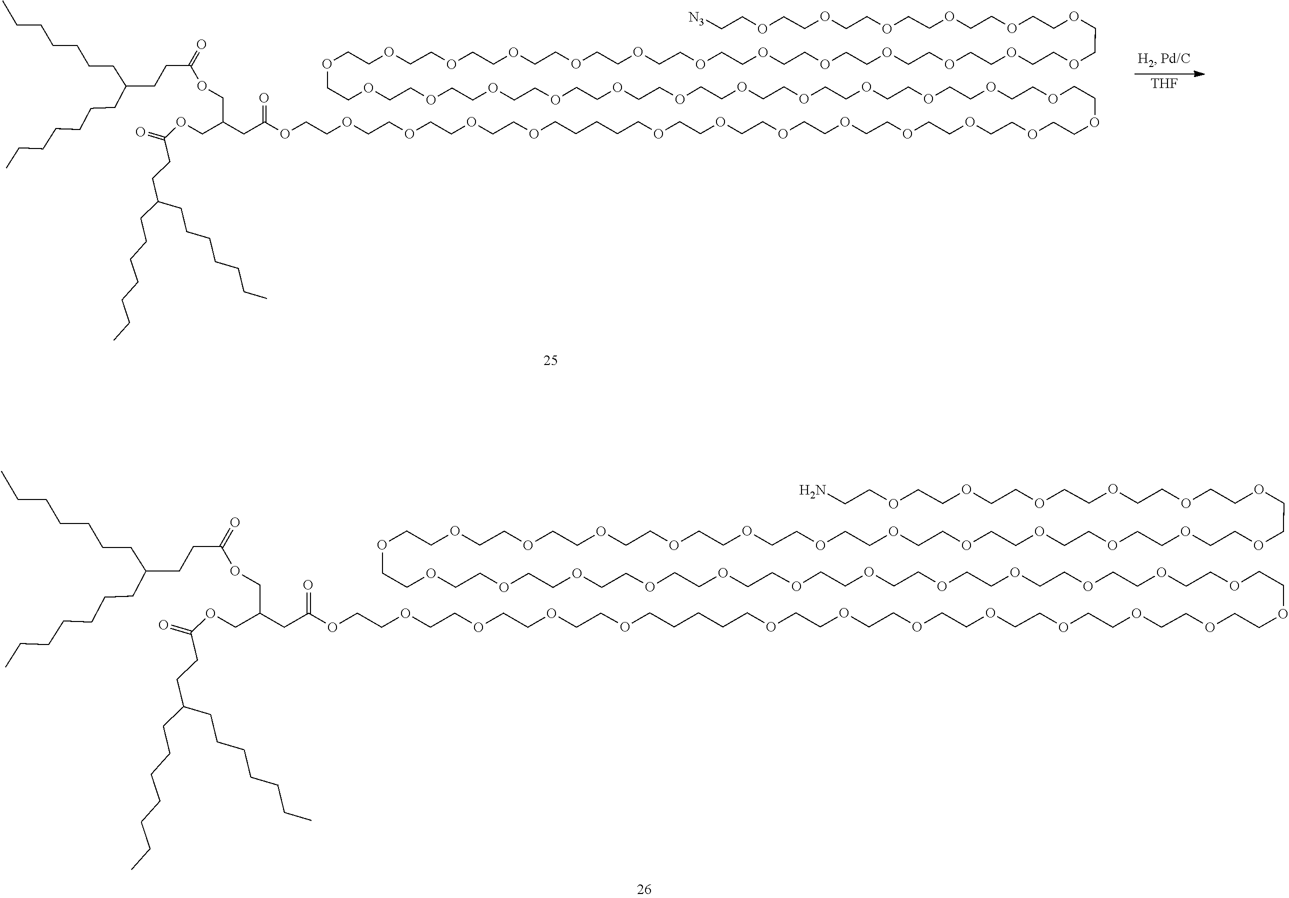

To a solution of 25 (200 mg, 0.076 mmol) in THE (100 mL) was added 10% Pd/C (40 mg). The system was evacuated and refilled with nitrogen (3×), then was placed under a balloon of hydrogen. Stirring under hydrogen was continued for 4 hours, then the solids were removed by filtration through a pad of Celite, the filter cake was rinsed with THE (20 mL) and the solvent was removed in vacuo to yield amine 26 (195 mg, 0.075 mmol, 98%) as an off-white waxy-solid.

HRMS: Calcd. for C₁₂₉H₂₅₇NO₄₉+H⁺2605.77. Found: 2605.77.

### Example 35. Synthesis of PEG-Bromomaleimide Amide 27

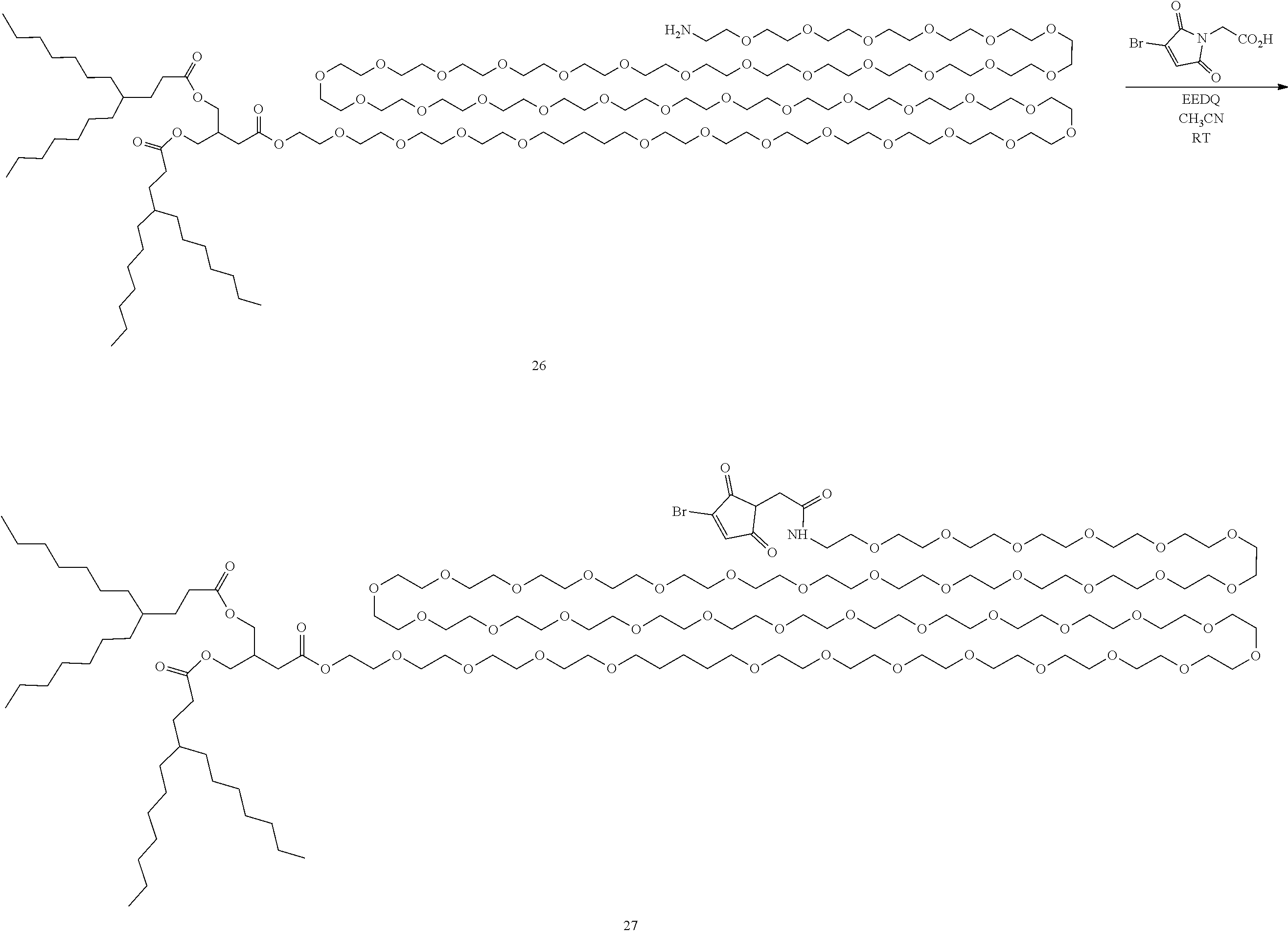 [SMILES: CCCCCCCC(CCCCCCC)CCC(=O)OCC(COC(=O)CCC(CCCCCCC)CCCCCCC)CC(=O)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCN ; O=C(O)CN1C(=O)C=C(Br)C1=O ; CCCCCCCC(CCCCCCC)CCC(=O)OCC(COC(=O)CCC(CCCCCCC)CCCCCCC)CC(=O)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CC1C(=O)C=C(Br)C1=O]

A solution of 2-(3-bromo-2,5-dioxo-2,5-dihydro-1H-pyrrol-1-yl)acetic acid (11.41 mg, 0.049 mmol) and ethyl 2-ethoxy-2H-quinoline-1-carboxylate (EEDQ, 14.25 mg, 0.058 mmol) in CH₃CN (15 mL) was stirred for 1 hour at room temperature under nitrogen, then 26 (150 mg, 0.058 mmol) was added in one portion. The resulting solution was stirred for 12 hours at room temperature, then the solvent was removed in vacuo to give crude 27 as a sticky yellow, semi-solid. Crude 22 was purified by Flash-Prep-HPLC (Xbridge BEH Phenyl column, gradient i-PrOH—CH₃CN-MeOH (30:20:50)/MeOH—H₂O-(70:30)-0.1% formic acid 80:20 to 85:15). Fractions containing 27 were pooled and the solvent was removed by lyophilization to provide 27 (30 mg, 0.0106 mmol, 18%) as an off-white solid.

¹H-NMR (400 MHz, CDCl₃): δ 6.94 (s, 1H), 6.90 (brs, 1H), 4.28 (s, 2H), 4.18 (m, 4H), 3.54-3.85 (176H), 3.48 (m, 2H), 2.34 (t, J=7.20H, 4H), 1.59 (m, 4H), 1.06-1.48 (54H), 0.90 (m, 12H); HRMS: Calcd. for C₁₃₅H₂₅₉BrN₂O₅₂+Na⁺2842.68/2844.68, Found 2842.68/2844.66.

### Example 36. Synthesis of PEG-Azide 29

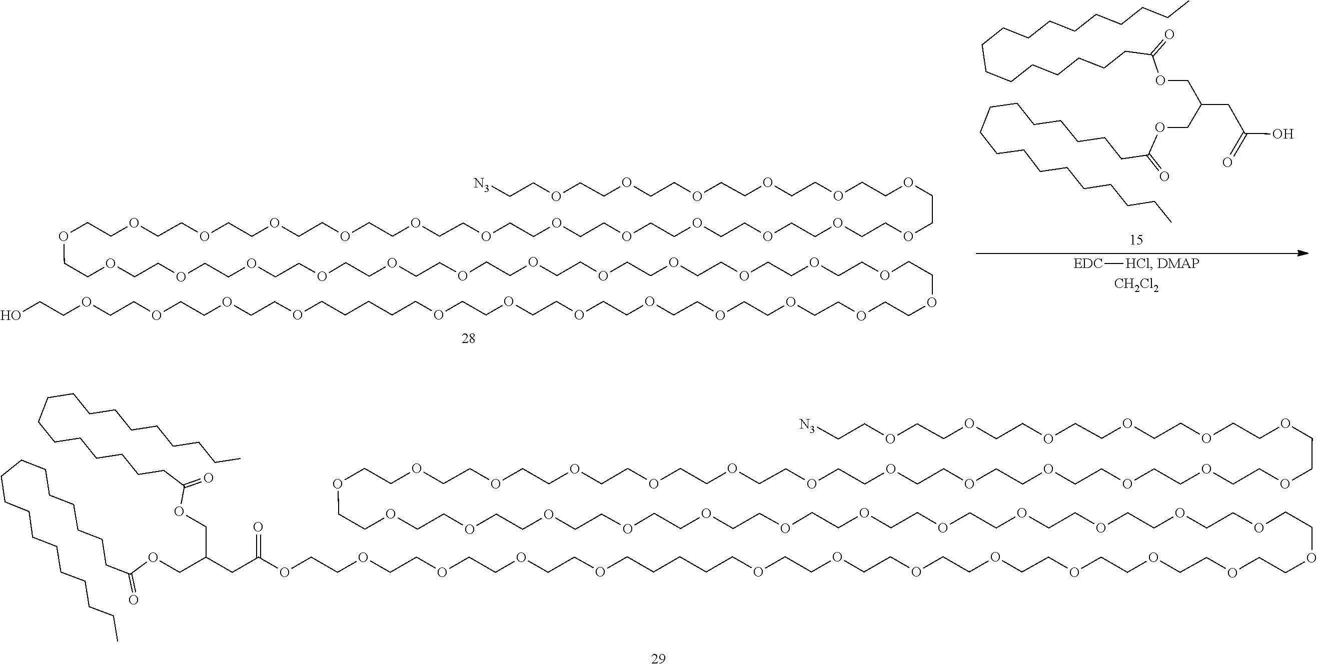

To a solution of PEG-2000 azide 28 (Sigma-Aldrich #900936, 3.00 g, 1.481 mmol) in CH₂Cl₂ (60 mL), under nitrogen, was added in order 15 (1.19 g, 1.787 mmol), DMAP (0.11 g, 0.889 mmol), and EDC-HCl (0.43 g, 2.24 mmol). The mixture was stirred for 16 hours at room temperature, then the solvent was removed in vacuo to give crude 29 as an off-white semi-solid. Crude 29 was dissolved in CH₂Cl₂ (20 mL), then silica gel (10 g, type: ZCX-2, 100-200 mesh) was added to the solution and the solvent was removed in vacuo to afford crude 29 impregnated on silica gel. The dry silica gel was placed onto a gravity column of silica gel (150 g, type: ZCX-2, 100-200 mesh, packed with CH₂Cl₂), and the resulting column was eluted with a gradient of CH₂Cl₂:MeOH (98:2 to 85:15). Fractions containing 29 were pooled and

HRMS: Calcd. for C₁₃₁H₂₅₉N₃O₅₀+Na⁺2695.76, Found 2695.76.

### Example 37. Synthesis of PEG-Amine 30

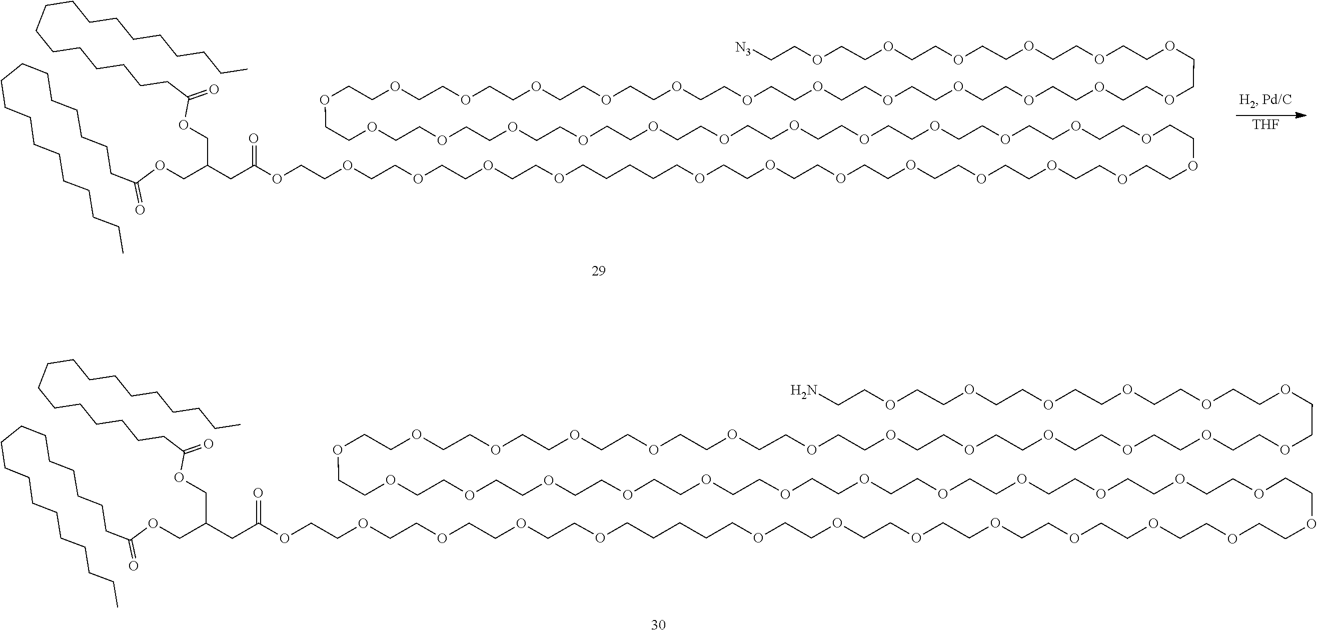 [SMILES: CCCCCCCCCCCCCCCCCC(=O)OCC(COC(=O)CCCCCCCCCCCCCCCCC)CC(=O)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCN=[N+]=[N-] ; CCCCCCCCCCCCCCCCCC(=O)OCC(COC(=O)CCCCCCCCCCCCCCCCC)CC(=O)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCN ; "THF"]

To a solution of 29 (0.500 g, 0.187 mmol) in THF (50 mL), under nitrogen, was added 10% Pd/C (100 mg). The system was evacuated and refilled with nitrogen (3×), then the mixture was placed under a balloon of hydrogen and was stirred for 4 hours. The solids were removed by filtration through a pad of Celite, the filter cake was rinsed with THE (25 mL), and the solvent was removed in vacuo to give 30 (0.430 g, 0.162 mmol, 87%) as an off-white solid.

HRMS: Calcd. for C₁₃₁H₂₅₉NO₅₀+Na⁺2647.78, Found 2647.79.

### Example 38. Synthesis of PEG-Bromomaleimide Amide 31

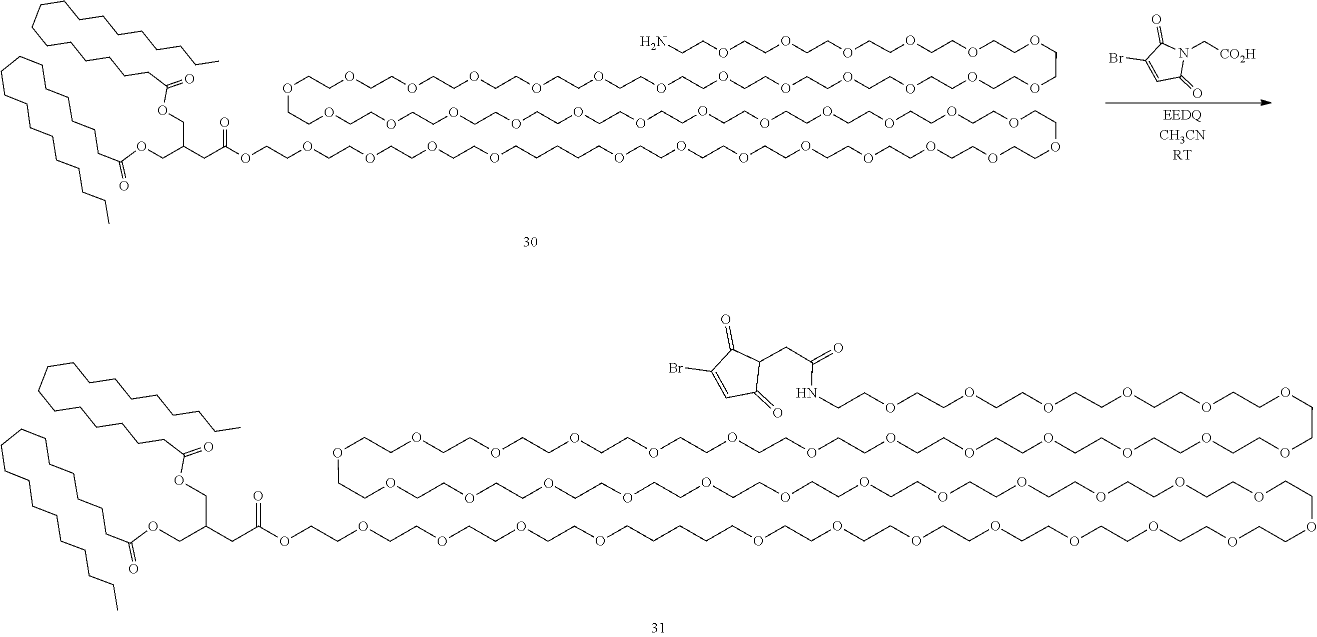 [SMILES: CCCCCCCCCCCCCCCCCC(=O)OCC(COC(=O)CCCCCCCCCCCCCCCCC)CC(=O)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCN ; O=C(O)CN1C(=O)C=C(Br)C1=O ; CCCCCCCCCCCCCCCCCC(=O)OCC(COC(=O)CCCCCCCCCCCCCCCCC)CC(=O)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CC1C(=O)C=C(Br)C1=O]

A solution of 2-(3-bromo-2,5-dioxo-2,5-dihydro-1H-pyrrol-1-yl)acetic acid (34.02 mg, 0.145 mmol) and ethyl 2-ethoxy-2H-quinoline-1-carboxylate (EEDQ, 42.48 mg, 0.172 mmol) in CH₃CN (50 mL) was stirred for 1 hour at room temperature under nitrogen, then 30 (500 mg, 0.132 mmol) was added in one portion. The resulting solution was stirred for 12 hours at room temperature, then the solvent was removed in vacuo to give crude 31 as a sticky pale-yellow, semi-solid. Crude 31 was purified by Flash-Prep-HPLC (Xbridge BEH Phenyl column, gradient i-PrOH—CH₃CN-MeOH (30:20:50)/MeOH—H₂O-(70:30)-0.1% formic acid, 80:20 to 85:15). Fractions containing 31 were pooled and the solvent was removed by lyophilization to provide 31 (180 mg, 0.063 mmol, 48%) as an off-white solid.

¹H-NMR (400 MHz, CDCl₃): δ 6.94 (s, 1H), 6.90 (brs, 1H), 4.20-4.33 (4H), 4.07-4.17 (4H), 3.30-3.90 (170H), 2.56 (m, 2H), 2.43 (t, J=6.80H, 2H), 2.31 (t, J=7.20 Hz, 4H), 1.60 (m, 4H), 1.10-1.50 (60H), 0.88 (m, 6H); HRMS: Calcd. for C₁₃₇H₂₆₁BrN₂O₅₃+H⁺2862.71/2864.71, Found 2862.71/2864.73.

### Example 39. Synthesis of PEG-Azide 32

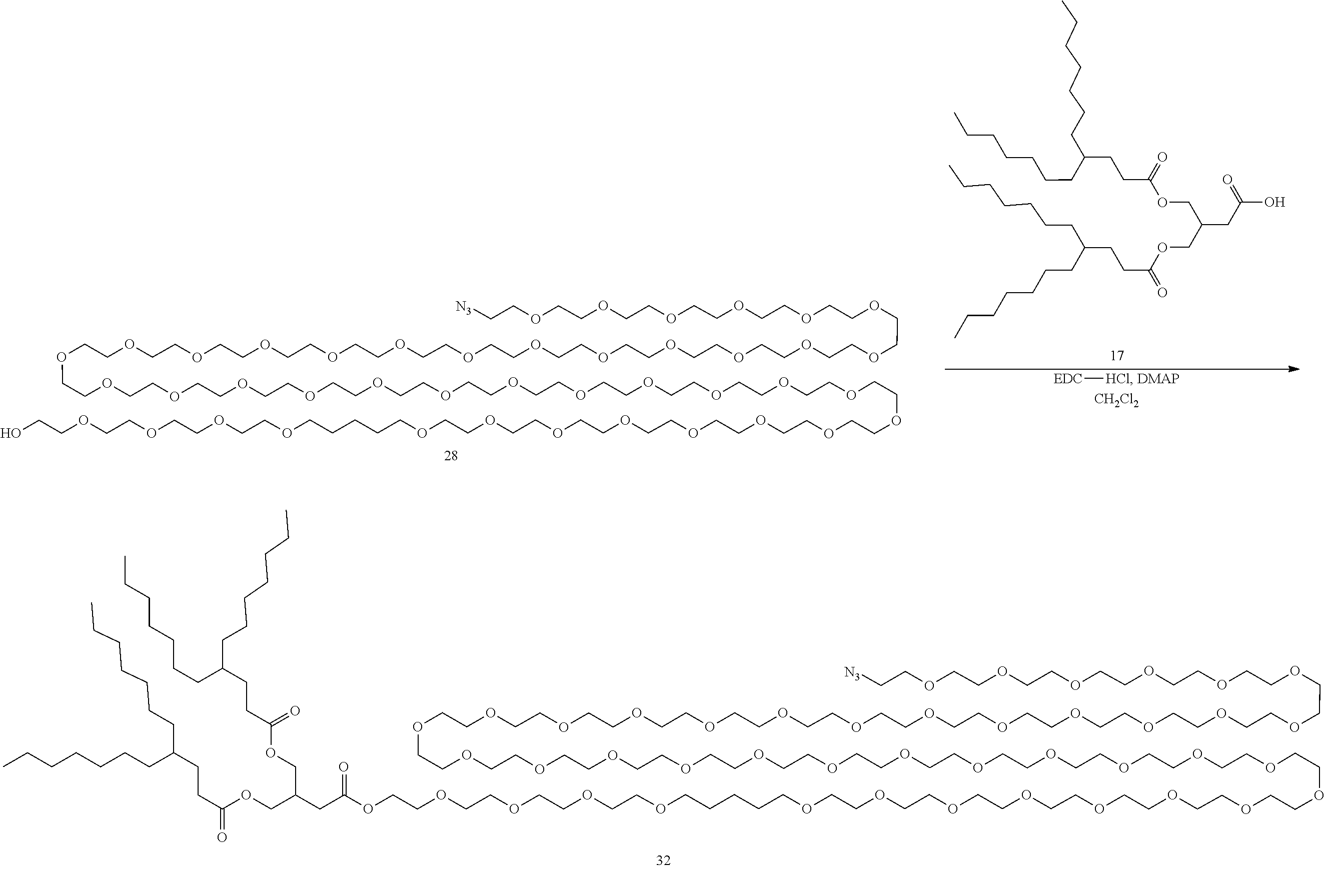

To a solution of PEG-2000 azide 28 (Sigma-Aldrich #900936, 1.00 g, 0.469 mmol) in CH₂Cl₂ (20 mL), under nitrogen, was added in order 17 (0.38 g, 0.563 mmol), DMAP (0.030 g, 0.281 mmol), and EDC-HCl (0.13 g, 0.704 mmol). The mixture was stirred for 16 hours at room temperature, then the solvent was removed in vacuo to give crude 32 as an off-white semi-solid. Crude 32 was dissolved in CH₂Cl₂ (20 mL), then silica gel (10 g, type: ZCX-2, 100-200 mesh) was added to the solution and the solvent was removed in vacuo to afford crude 32 impregnated on silica gel. The dry silica gel was placed onto a gravity column of silica gel (100 g, type: ZCX-2, 100-200 mesh, packed with CH₂Cl₂), and the resulting column was eluted with a gradient of CH₂Cl₂:MeOH (98:2 to 85:15). Fractions containing 32 were pooled and the solvent was removed in vacuo to provide 32 (1.10 g, 0.411 mmol, 88%) as an off-white solid.

HRMS: Calcd. for C₁₃₁H₂₅₇N₃O₅₀+H⁺2673.77, Found 2673.79.

### Example 40. Synthesis of PEG-Amine 33

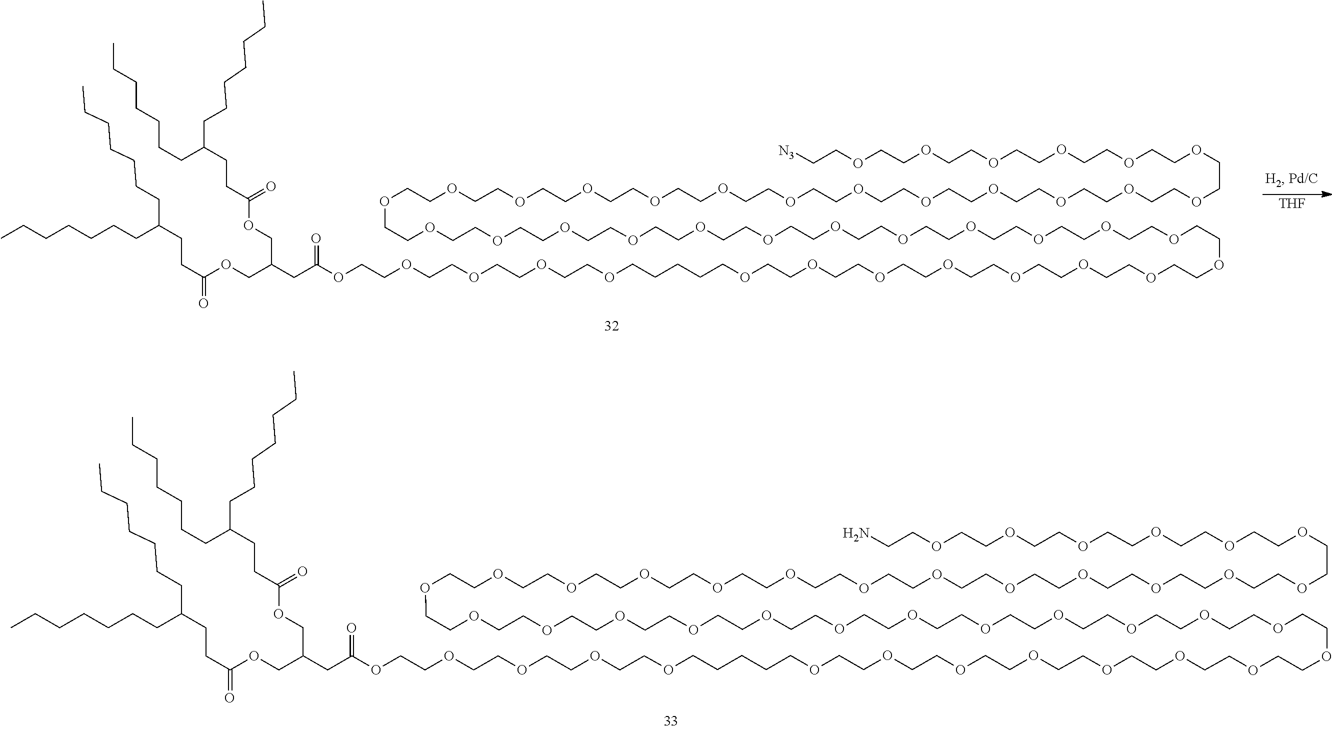

To a solution of 32 (1.10 g, 0.362 mmol) in THF (110 mL), under nitrogen, was added 10% Pd/C (200 mg). The system was evacuated and refilled with nitrogen (3×), then the mixture was placed under a balloon of hydrogen and was stirred for 4 hours. The solids were removed by filtration through a pad of Celite, the filter cake was rinsed with THF (25 mL), an5), then silica gel (30 g, type: ZCX-2, 100-200 mesh) was added to the solution and the solvent was removed in vacuo to afford crude 33 impregnated on silica gel. The dry silica gel was placed onto a gravity column of silica gel (150 g, type: ZCX-2, 100-200 mesh, packed with CH₂Cl₂), and the resulting column was eluted with a gradient of CH₂Cl₂:MeOH (98:2 to 85:15). Fractions containing 33 were pooled and the solvent was removed in vacuo to provide 33 (0.380 g, 0.143 mmol, 40%) as an off-white solid.

HRMS: Calcd. for C₁₃₁H₂₅₉NO₅₀+H⁺2647.78, Found 2647.78.

### Example 41. Synthesis of PEG-Bromomaleimide Amide 34

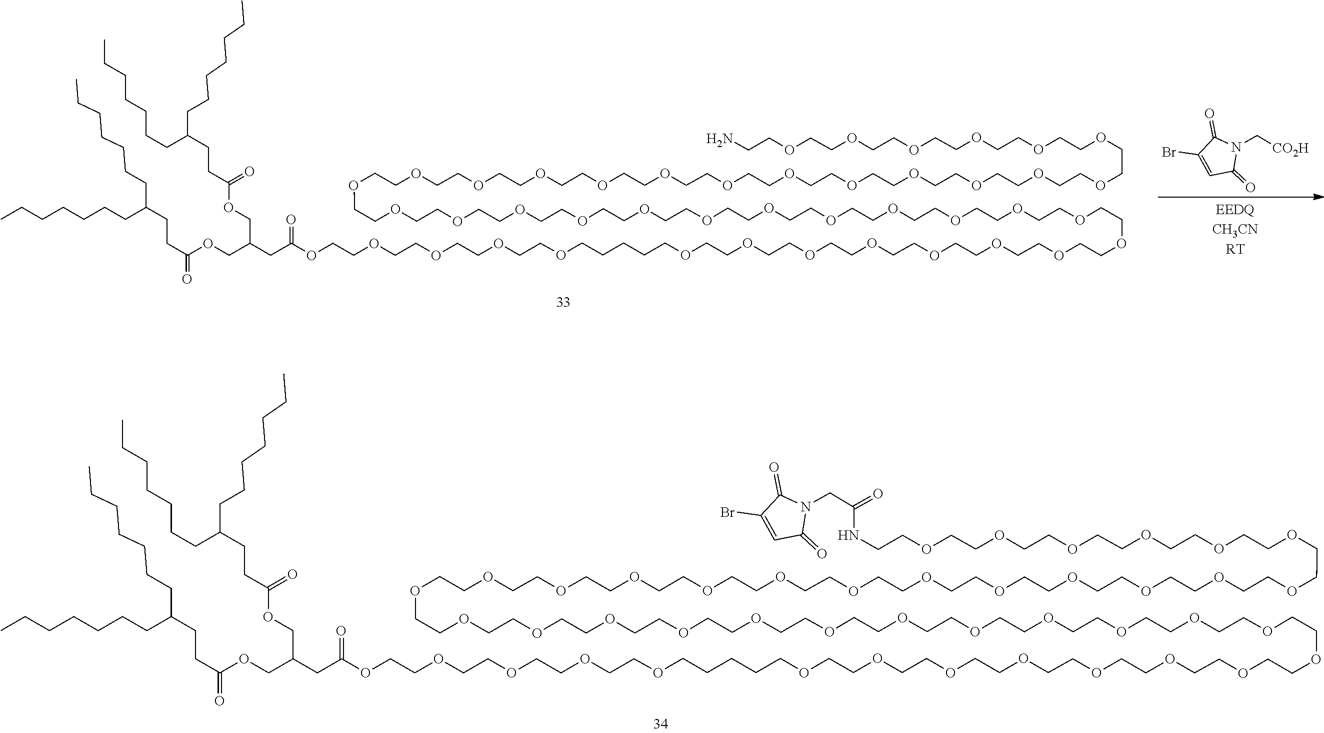 [SMILES: CCCCCCCC(CCCCCCC)CCC(=O)OCC(COC(=O)CCC(CCCCCCC)CCCCCCC)CC(=O)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCN ; O=C(O)CN1C(=O)C=C(Br)C1=O ; CCCCCCCC(CCCCCCC)CCC(=O)OCC(COC(=O)CCC(CCCCCCC)CCCCCCC)CC(=O)OCCOCCOCCOCCOCCCCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CN1C(=O)C=C(Br)C1=O]

A solution of 2-(3-bromo-2,5-dioxo-2,5-dihydro-1H-pyrrol-1-yl)acetic acid (14.46 mg, 0.062 mmol) and ethyl 2-ethoxy-2H-quinoline-1-carboxylate (EEDQ, 18.06 mg, 0.73 mmol) in CH₃CN (5 mL) was stirred for 1 hour at room temperature under nitrogen, then 33 (160 mg, 0.060 mmol) was added in one portion. The resulting solution was stirred for 12 hours at room temperature, then the solvent was removed in vacuo to give crude 34 as a sticky pale-yellow, semi-solid. Crude 34 was purified by Flash-Prep-HPLC (Xbridge BEH Phenyl column, gradient i-PrOH—CH₃CN-MeOH (30:20:50)/MeOH—H₂O-(70:30)-0.1% formic acid, 80:20 to 85:15). Fractions containing 34 were pooled and the solvent was removed by lyophilization to provide 34 (49 mg, 0.017 mmol, 25%) as an off-white solid.

¹H-NMR (400 MHz, CDCl₃): δ 6.94 (brs, 1H), 6.94 (s, 1H), 4.20-4.33 (4H), 4.07-4.17 (4H), 3.35-3.80 (177H), 2.47 (m, 2H), 2.30 (m, 4H), 1.60 (m, 4H), 1.10-1.50 (50H), 0.89 (m, 12H); HRMS: Calcd. for C₁₃₇H₂₆₁BrN₂O₅₃+H⁺2863.71/2865.71, Found 2863.71/2865.71.

### Example 42. LNP Encapsulation of mRNA

The ability to incorporate various of the disclosed PEG-lipids into LNP encapsulating mRNA was assessed using mRNA encoding the fluorescent marker mCherry.

mCherry mRNA was synthesized by T7 RNA polymerase mediated in vitro transcription (IVT) of a linearized DNA template, using full substitution of uridine with N1-Methylpseudouridine. A Cap1 structure was added to the 5′ end of the mRNA co-transcriptionally and a 3′ polyadenosine tail was encoded by the DNA template. Post IVT, mRNA was purified using a two-step chromatography process using OligoDT affinity chemistry for bulk capture and ion-pair reverse phase chemistry to remove residual impurities.

The mRNA was encapsulated in LNP using a self-assembly process in which an aqueous solution of mRNA at pH=3.5 is rapidly mixed with a solution of lipids dissolved in ethanol, then followed by stepwise phosphate and Tris buffer dilution and tangential flow filtration (TFF) purification. LNP composition in this study was: an ionizable cationic lipid of Formula 1a as shown below/distearoylphosphatidylcholine/cholesterol/PEG-lipid (50:10:38.5:1.5 mol/mol) and were encapsulated at an N/P ratio (the ratio of positively-chargeable lipid amine (N=nitrogen) groups to negatively-charged nucleic acid phosphate (P) groups) at 6. LNPs were frozen at −80° C. LNP were made in which the PEG-lipid was DMG-PEG2000, as a benchmark, or one of Compounds VI-7, VI-8, B-46, or B-47. The diameter of the nanoparticles was measured by dynamic light scattering using a Zetasizer Nano ZS (Malvern Instruments Ltd., Malvern, UK) instrument. Size measurement was carried out in pH 7.4 Tris buffer at 25° C. in relevant disposable capillary cells. A non-invasive back scatter system (NIBS) with a scattering angle of 173° was used for size measurements.

TABLE 1
Physical-chemical properties of the LNP

| | Hydrodynamic | | Encapsulation |
|---|---|---|---|
| PEG-lipid | diameter (nm) | PDI* | Efficiency (%) |
| DMG-PEG2000 | 82 | 0.03 | 96 |
| VI-7 | 97 | 0.10 | 72 |
| VI-8 | 178 | 0.26 | 84 |
| B-46 | 88 | 0.04 | 85 |
| B-47 | 98 | 0.12 | 68 |

*polydispersity index

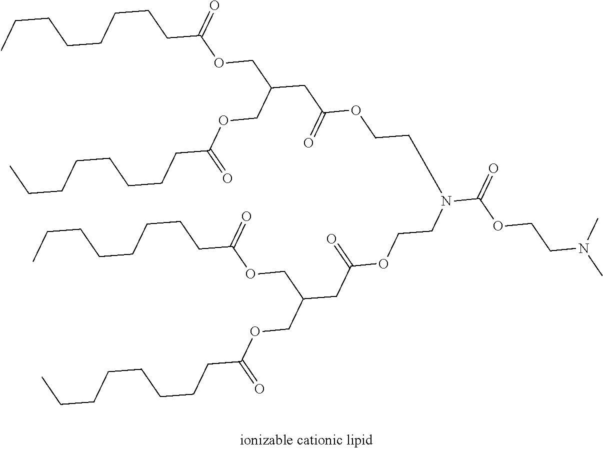 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN(C)C]

All four of Compounds VI-7, VI-8, B-46, or B-47 (the experimental PEG-lipids) formed LNP, with VI-8-containing LNP displaying physical-chemical properties essentially the same as benchmark LNP formed with DMG-PEG2000 as the PEG-lipid.

### Example 43. Transfection of HEK293F Cells

The ability of the LNP formed in the preceding Example to transfect HEK293F cells, a human embryonic kidney cell-derived cell line, with the mCherry mRNA was assessed. Viral Production Cells (Gibco Catalog number: A35347), a derivative of the HEK 293F cell line adapted to a chemically-defined, serum-free and protein-free medium (LV-MAX™ Production Medium; Gibco Catalog number: A3583401) were grown in suspension, sedimented, resuspended at about 1×10⁶ cells/mL, and 200 μL distributed to the wells of a 96-well U-bottom plate. Frozen LNP were thawed and diluted to 100 μg mRNA/mL with sterile water for injection. An appropriate volume of LNP was added to provide 0, 0.3, 0.6, or 2 μg RNA per well in duplicate and mixed by re-pipetting. The cells were then incubated for 1 hour at 37° C. in a CO₂ incubator, washed three times with phosphate buffered saline, resuspended in 400 μL of medium in a deep-well 96-well plate, and incubated at 37° C. in a CO₂ incubator on an orbital shaker at 900 RPM.

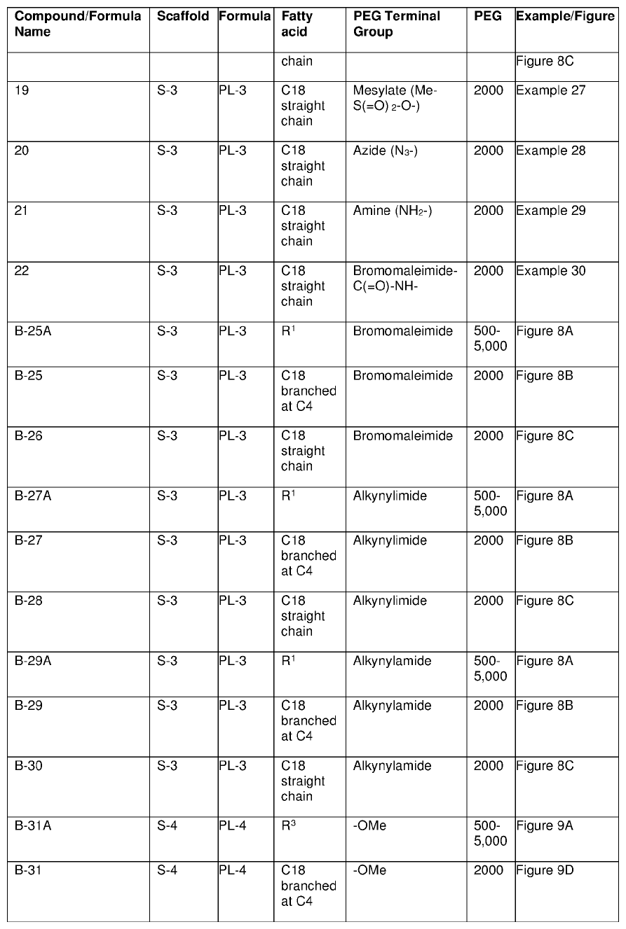

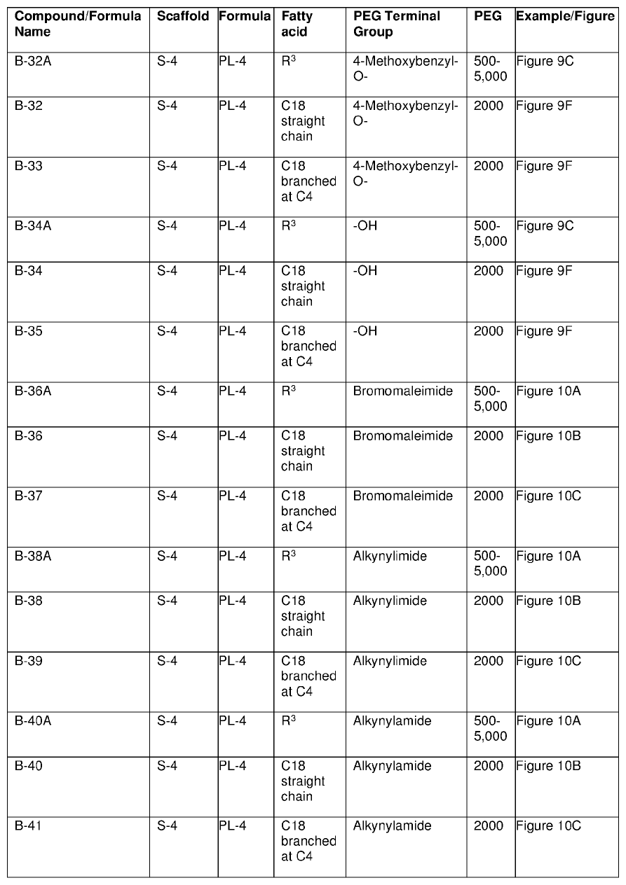

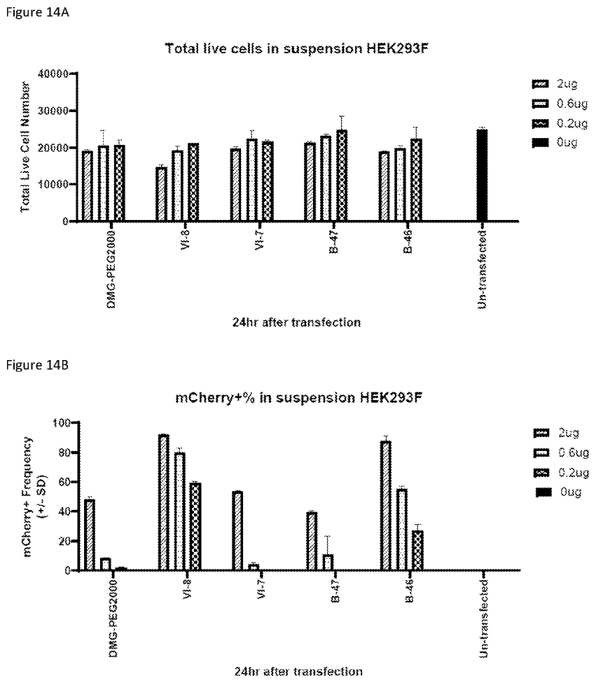

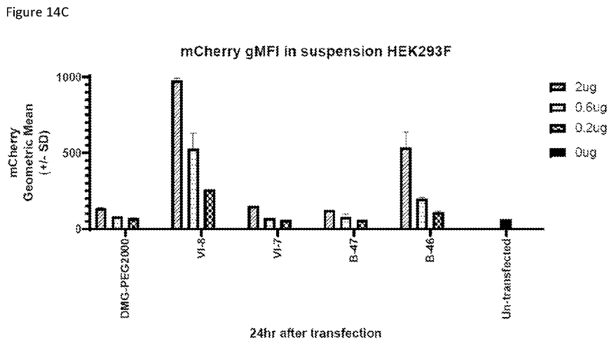

Twenty-four hours after addition of the LNP to the cells they were stained with Aqua Live/Dead (Thermo: catalog L34965) to assess cell viability. Transfection rate and expression level in the transfected cells was assessed by flow cytometry based on mCherry fluorescence. As seen in FIG. 14A, all of the LNP caused a small reduction in cell number as compared to untransfected cells, but the decreases were acceptable and generally comparable among the various LNP including the benchmark.

As seen in FIG. 14B, the greatest transfection rate was achieved with the LNP comprising the PEG-lipids VI-8 or B-46, achieving nearly 90% transfection of live cells at their highest LNP doses tested and still substantial levels of transfection at the lower doses. The LNP in which the PEG-lipid was VI-7 or B-47 performed comparably to the benchmark LNP achieving in the range of 40-60% transfection of live cells at their highest LNP doses tested, but minimal transfection at the lower doses. The results for expression level, seen in FIG. 14C, showed a similar pattern with the LNP comprising the PEG-lipids VI-8 or B-46 provided substantially higher expression levels of mCherry than the benchmark LNP whereas LNP in which the PEG-lipid was VI-7 or B-47 performed comparably.

Structurally, all of the experimental PEG-lipids have C₁₈ fatty acid ester tails; in VI-8 and B-46 the tails are straight-chain while in VI-7 and B-47 the tails are branched. Compounds B-46 and B-47 are built on scaffold S1 while VI-7 and VI-8 are built on scaffold S3 (a symmetric glycerol scaffold). In contrast, the benchmark PEG-lipid for this Example, DMG-PEG2000 has C₁₄ straight-chain fatty acid ester tails built on an asymmetric glycerol scaffold. Accordingly, the data indicate that the straight-chain PEG-lipids perform better than their branched chain analogs or the benchmark lipid's shorter straight-chain tails. The two symmetric scaffolds, S1 and S3, appear to perform similarly.

### Example 44. LNP Encapsulation of mRNA and Binding Moiety Conjugation

The ability to incorporate various of the disclosed functionalized PEG-lipids into LNP encapsulating mRNA and conjugation to a binding moiety was assessed using mRNA encoding the fluorescent marker mCherry and an anti-CD5 antibody. Compounds B-3 and B-25 were used as the experimental functionalized PEG-lipids and DSPE-PEG2000 was used as a benchmark functionalized PEG-lipid. Compounds B-3 and B-25 are analogues of B-47 and VI-7, respectively, in which the 4-methoxybenzyloxyl group has been replaced with a bromomaleimide group.

CleanCap® mCherry 5-methoxyuridine (5moU) mRNA (L-7203) was purchased from TriLink. mRNA was encapsulated in LNP using a self-assembly process in which an aqueous solution of mRNA at pH=3.5 is rapidly mixed with a solution of lipids dissolved in ethanol, then followed by stepwise phosphate and Tris buffer dilution and TFF purification. LNP composition in this study was: an ionizable cationic lipid as shown in Example 42/distearoylphosphatidylcholine/cholesterol/DMG-PEG 2000/functionalized PEG-lipid (50:10:38.5:1.4:0.1 mol/mol) and were encapsulated at an N/P ratio (the ratio of positively-chargeable polymer amine (N=nitrogen) groups to negatively-charged nucleic acid phosphate (P) groups) at 6. The hydrodynamic diameter of the nanoparticles was measured by dynamic light scattering using a Zetasizer Nano ZS (Malvern Instruments Ltd., Malvern, UK) instrument.

Next, an anti-CD5 mAb was conjugated to the above LNP to generate tLNP. Purified rat anti-mouse CD5 antibody, clone 53-7.3 (BioLegend), was coupled to LNP via N-succinimidyl S-acetylthioacetate (SATA)-maleimide conjugation chemistry. Briefly, LNPs with DSPE-PEG2000-maleimide incorporated were formulated and stored at 4° C. on the day of conjugation. The antibody was modified with SATA (Sigma-Aldrich) to introduce sulfhydryl groups at accessible lysine residues allowing conjugation to maleimide. SATA was deprotected using 0.5 M hydroxylamine followed by removal of the unreacted components by G-25 Sephadex Quick Spin Protein columns (Roche Applied Science, Indianapolis, IN). The reactive sulfhydryl group on the antibody was then conjugated to maleimide moieties on the LNPs using thioether conjugation chemistry. Purification was performed using Sepharose CL-4B gel filtration columns (Sigma-Aldrich). tLNPs (LNPs conjugated with a targeting antibody) were frozen at −80° C. Others have conjugated antibody to free functionalized PEG-lipid and then incorporated the conjugated lipid into pre-formed LNP. However, we have found that the present procedure is more controllable and produces more consistent results. The same procedures were used for the LNP incorporating the bromomaleimide functionalized PEG-lipids without any optimization for the differing functional group.

mRNA content was determined using a Quant-iT™ RiboGreen RNA assay kit (Invitrogen™). Encapsulation efficiency was calculated by determining the unencapsulated mRNA content by measuring the fluorescence intensity (Fi) upon the addition of RiboGreen reagent to the LNP and comparing this value to the total fluorescence intensity (Ft) of the RNA content that is obtained upon lysis of the LNPs by 1% Triton X-100, where % encapsulation=(Ft−Fi)/Ft×100).

The particle size (hydrodynamic diameter) and polydispersity index of the targeted lipid nanoparticles were determined using dynamic light scattering (DLS) on a Malvern Zetasizer Nano ZS (Malvern Instruments, Worcestershire, UK). Size measurement was carried out in pH 7.4 Tris buffer at 25° C. in relevant disposable capillary cells. A non-invasive back scatter system (NIBS) with a scattering angle of 173° was used for size measurements.

TABLE 2
Physical-chemical properties of the tLNP

| | Hydrodynamic | | Encapsulation |
|---|---|---|---|
| Functionalized PEG-lipid | diameter (nm) | PDI* | Efficiency (%) |
| DSPE-PEG-Mal | 90 | 0.1 | 96 |
| B-25 | 98 | 0.2 | 91 |
| B-3 | 97 | 0.1 | 90 |

*polydispersity index

The size, polydispersity, and encapsulation efficiency of the tLNP were all acceptable or better and comparable amongst all three preparations.

### Example 45. Targeted Transfection of T Cells

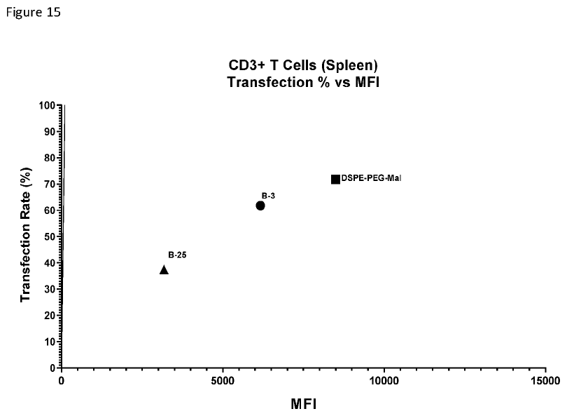

To assess the performance of tLNP described in the preceding Example the tLNP were used to transfect mouse T cells in tissue culture. Mouse splenic T cells were isolated from mechanically dissociated mouse spleens using a standard T cell isolation kit (Stem Cell Technologies #19851). Isolated T cells were cultured in complete RPMI medium supplemented with murine interleukin-2 in the presence of CD3/CD28 T cell activation beads (Gibco #11453D) for 3 days. Following activation, T cells were magnetically separated from the activation beads and transferred to a 96-well plate at a concentration of 2×10⁵ cells per well in 100 μL of complete RPMI medium. The tLNP formulations were diluted to 100 μg/mL and 6 μL (0.6 μg) of tLNP was added to each well of cells to be tested. Cells were incubated with tLNPs at 37° C. for 1 hour before tLNPs were washed away by centrifuging the plate, removing the supernatant, and replacing with fresh medium. Transfected cells were then returned to the incubator overnight. The next day, cells were washed and resuspended in stain buffer containing fluorescently tagged antibodies against T cells markers for 30 minutes before a final wash. After washing, cells were resuspended in stain buffer and run on the Novocyte Quanteon flow cytometer to detect mCherry expression as well as murine T cell markers. Results for CD3⁺ T cells are depicted in FIG. 15. All three of the tLNP preparations successfully delivered the mCherry mRNA into T cells where it was expressed.

### Example 46. Further Embodiments

Further embodiments are provided below.

Embodiment 1. A polyethylene glycol (PEG)-lipid having the structure of Formula PL-1

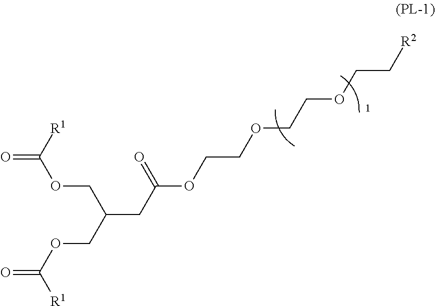

wherein R¹ is C₁₃-C₁₉ alkyl that is either straight-chain or symmetric branched-chain,
R² is —OH, —OCH₃, —O-benzyl, 4-methoxybenzyloxyl, maleimide, azide, alkynyl, dibenzocyclooctyne (DBCO), bromomaleimide, bromomaleimide amide, alkynylimide, or alkynylamide, and
n≈20 to 115.

Embodiment 2. The PEG-lipid of Embodiment 1 having the structure of Compound B-1, B-43, B-45, B-47 B-2, B-42, B-44, or B-46.

Embodiment 3. A PEG-lipid, having the structure of Formula PL-2

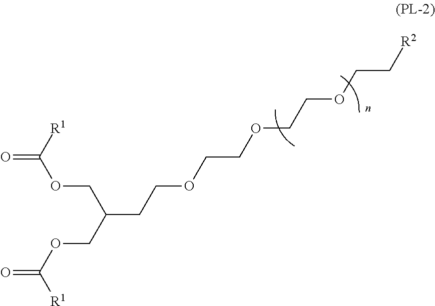

wherein R¹ is C₁₃-C₁₉ alkyl that is either straight-chain or symmetric branched-chain,
R² is —OH, —OCH₃, —O-benzyl, 4-methoxybenzyloxyl, maleimide, azide, alkynyl, DBCO, bromomaleimide, bromomaleimide amide, alkynylimide, or alkynylamide, and
n≈20 to 115.

Embodiment 4. The PEG-lipid of Embodiment 3 having the structure of Compound B-9 or B-10.

Embodiment 5. The PEG-lipid of Embodiment 3 having the structure of Compound B-11, B-12, B-13, or B-14.

Embodiment 6. A PEG-lipid having the structure of Formula PL-3

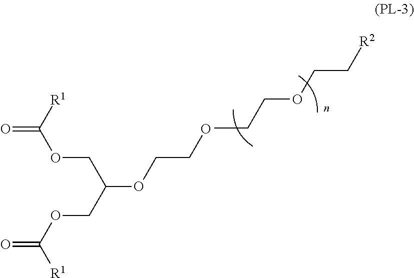

wherein R¹ is C₁₃-C₁₉ alkyl that is either straight-chain or symmetric branched-chain,
R² is —OH, —OCH₃, —O-benzyl, 4-methoxybenzyloxyl, maleimide, azide, alkynyl, DBCO, bromomaleimide, bromomaleimide amide, alkynylimide, or alkynylamide, and
n≈10 to 115.

Embodiment 7. The PEG-lipid of Embodiment 6 having the structure of Compound B-23 or B-24.

Embodiment 8. The PEG-lipid of Embodiment 6 having the structure of Compound VI-7, B-21, VI-8, or B-22.

Embodiment 9. A PEG-lipid having the structure of Formula PL-4

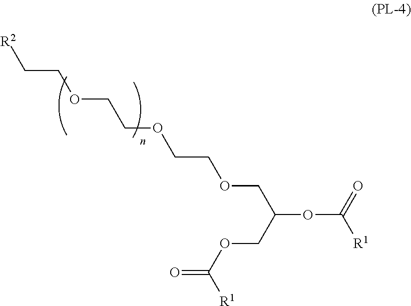

wherein R¹ is C₁₃-C₁₉ alkyl that is either straight-chain or a symmetric branched-chain, R² is —OH, —OCH₃, —O-benzyl, 4-methoxybenzyloxyl, maleimide, azide, alkynyl, DBCO, bromomaleimide, bromomaleimide amide, alkynylimide, or alkynylamide, and
n≈10 to 115.

Embodiment 10. The PEG-lipid of Embodiment 9 having the structure of Compound B-31.

Embodiment 11. The PEG-lipid of Embodiment 9 having the structure of Compound B-33 or B-35.

Embodiment 12. The PEG-lipid of any one of Embodiments 1-11 having branched chain alkyl fatty acid esters wherein the branch is at the 3, 4, 5, 6, or 7 position of the fatty acid,

Embodiment 13. A functionalized PEG-lipid comprising a bromomaleimide, bromomaleimide amide, alkynylamide, or alkynylimide appended to a terminal hydroxyl end of the PEG moiety.

Embodiment 14. The functionalized PEG-lipid of Embodiment 13 having the structure of Compound B-3, B-4, B-5, B-6, B-7, B-8, B-15, B-16, B-17, B-18, B-19, B-20, B-25, B-26, B-27, B-28, B-29, B-30, B-36, B-37, B-38, B-39, B-40, or B-41.

Embodiment 15. The PEG-lipid of any one of Embodiments 1-14, wherein the PEG-moiety comprises a PEG in a size range of PEG-500 to PEG-5000.

Embodiment 16. A lipid nanoparticle (LNP) comprising the PEG-lipid of any one of Embodiments 1-15.

Embodiment 17. The LNP of Embodiment 16, further comprising one or more of a phospholipid, an ionizable cationic lipid, a sterol, a co-lipid, and a further PEG-lipid, or combinations thereof.

Embodiment 18. The LNP of Embodiment 17, wherein the phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof.

Embodiment 19. The LNP of Embodiment 17 or 18, wherein the sterol comprises cholesterol, campesterol, sitosterol, or stigmasterol, or combinations thereof.

Embodiment 20. The LNP of any one of Embodiments 17-19, wherein the co-lipid comprises cholesterol hemisuccinate (CHEMS) or a quaternary ammonium headgroup containing lipid.

Embodiment 21. The LNP of Embodiment 20, wherein the quaternary ammonium headgroup containing lipid comprises 1,2-dioleoyl-3-trimethylammonium propane (DOTAP), N-(1-(2,3-dioleyloxy)propyl)-N,N,N-trimethylammonium (DOTMA), or 3p-(N-(N′,N′-Dimethylaminoethane)carbamoyl)cholesterol (DC-Chol), or combinations thereof.

Embodiment 22. The LNP of any one of Embodiments 14-21, wherein the further PEG-lipid comprises DMG-PEG2000 (1,2-dimyristoyl-glycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof.

Embodiment 23. The LNP of any one of Embodiments 16-22, wherein the PEG-lipid is a functionalized PEG-lipid.

Embodiment 24. The LNP of Embodiment 23, wherein the functionalized PEG-lipid is conjugated with a binding moiety.

Embodiment 25. The LNP of Embodiment 24, wherein the binding moiety comprises an antigen-binding domain of an antibody.

Embodiment 26. The LNP of any one of Embodiments 16-25, comprising 0.1 to 5% PEG-lipid.

Embodiment 27. The LNP of any one of Embodiments 17-26, comprising 40 to 60 mol % ionizable cationic lipid.

Embodiment 28. The LNP of any one of Embodiments 17-27, comprising 7 to 30 mol % phospholipid.

Embodiment 29. The LNP of any one of Embodiments 17-28, comprising 20 to 45 mol % sterol.

Embodiment 30. The LNP of any one of Embodiments 17-29, comprising 1 to 30 mol % co-lipid.

Embodiment 31. The LNP of any one of Embodiments 17-20, comprising 0 to 5 mol % further PEG-lipid.

Embodiment 32. The LNP of any one of Embodiments 16-31, further comprising a nucleic acid.

Embodiment 33. The LNP of Embodiment 32, wherein the weight ratio of total lipid to nucleic acid is 10:1 to 50:1.

Embodiment 34. The LNP of Embodiment 32 or 33, comprising mRNA.

Embodiment 35. A method of delivering a nucleic acid into a cell comprising contacting the cell with the LNP of any one of Embodiments 32-34.

1. A polyethylene glycol (PEG)-lipid having the structure of Formula PL-1 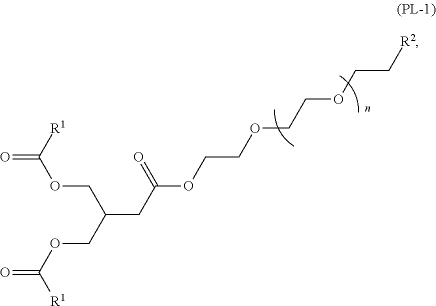
or the structure of Formula PL-2 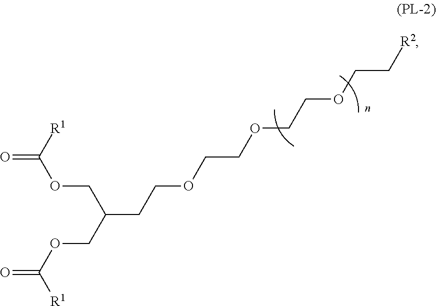
or the structure of Formula PL-3 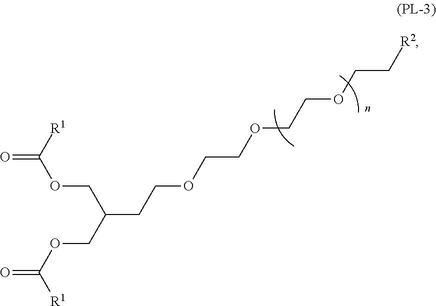
or the structure of Formula PL-4 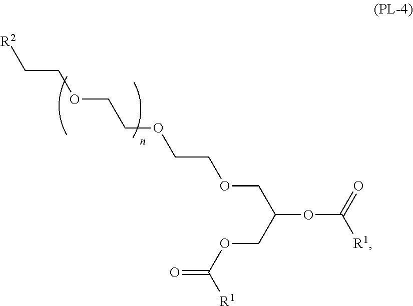
wherein R¹ is C₁₃-C₁₉ alkyl that is either straight-chain or symmetric branched-chain,
R² is —OH, —OCH₃, —O-benzyl, 4-methoxybenzyloxyl, maleimide, azide, alkynyl, dibenzocyclooctyne (DBCO), bromomaleimide, bromomaleimide amide, alkynylimide, or alkynylamide, and
n≈10 to 115.

2. The PEG-lipid of claim 1 having the structure of Compound B-1, B-43, B-45, B-47 B-2, B-42, B-44, or B-46.

3. (canceled)

4. The PEG-lipid of claim 1 having the structure of Compound B-9 or B-10.

5. The PEG-lipid of claim 1 having the structure of Compound B-11, B-12, B-13, or B-14.

6. (canceled)

7. The PEG-lipid of claim 1 having the structure of Compound B-23 or B-24.

8. The PEG-lipid of claim 1 having the structure of Compound VI-7, B-21, VI-8, or B-22.

9. (canceled)

10. The PEG-lipid of claim 1 having the structure of Compound B-31.

11. The PEG-lipid of claim 1 having the structure of Compound B-33 or B-35.

12. The PEG-lipid of claim 1, wherein R¹ is a branched C₁₃-C₁₉ alkyl, wherein the branch is at the 3, 4, 5, 6, or 7 position of the fatty acid ester.

13. A functionalized PEG-lipid comprising a bromomaleimide, bromomaleimide amide, alkynylamide, or alkynylimide appended to a terminal hydroxyl end of the PEG moiety.

14. The functionalized PEG-lipid of claim 13 having the structure of Compound B-3, B-4, B-5, B-6, B-7, B-8, B-15, B-16, B-17, B-18, B-19, B-20, B-25, B-26, B-27, B-28, B-29, B-30, B-36, B-37, B-38, B-39, B-40, or B-41.

15. (canceled)

16. A lipid nanoparticle (LNP) comprising the PEG-lipid of claim 1.

17. The LNP of claim 16, further comprising one or more of a phospholipid, an ionizable cationic lipid, a sterol, a co-lipid, and a further PEG-lipid, or combinations thereof.

18.-22. (canceled)

23. The LNP of claim 16, wherein the PEG-lipid is further functionalized at the terminal hydroxyl end of the PEG moiety.

24. The LNP of claim 23, wherein the functionalized PEG-lipid is conjugated with a binding moiety.

25. (canceled)

26. The LNP of claim 17, comprising 0.1 to 5% PEG-lipid, 40 to 60 mol % ionizable cationic lipid, 7 to 30 mol % phospholipid, 20 to 45 mol % sterol, 1 to 30 mol % co-lipid, or 0 to 5 mol % further PEG-lipid.

27.-31. (canceled)

32. The LNP of claim 16, further comprising a nucleic acid.

33. (canceled)

34. The LNP of claim 32, comprising mRNA.

35. A method of delivering a nucleic acid into a cell comprising contacting the cell with the LNP of claim 32.

36. A method of delivering a nucleic acid into a cell comprising contacting the cell with the LNP of claim 24, wherein the LNP further comprises a nucleic acid.
